(19) United States
(12) Patent Application Publication
(10) Pub. No.: US 2022/0233471 A1
(43) Pub. Date: Jul. 28, 2022

(54) PRODRUG COMPOSITIONS AND METHODS OF TREATMENT

(71) Applicant: AQUESTIVE THERAPEUTICS, INC., WARREN, NJ (US)

(72) Inventors: Alexander Mark SCHOBEL, Vero Beach, FL (US); Daniel R. BARBER, Summit, NJ (US); Stephen Paul WARGACKI, Pittstown, NJ (US); Rajesh Kumar KAINTHAN, Tappan, NY (US); Stephanie M. VARJAN, Oradell, NJ (US); Malarvizhi DURAI, Clarksville, MD (US)

(73) Assignee: AQUESTIVE THERAPEUTICS, INC., WARREN, NJ (US)

(21) Appl. No.: 17/576,923

(22) Filed: Jan. 15, 2022

Related U.S. Application Data
(60) Provisional application No. 63/137,881, filed on Jan. 15, 2021.

(51) Int. Cl. A61K 31/137; A61K 9/00; A61M 15/00

(52) U.S. Cl. CPC A61K 31/137; A61M 15/003; A61M 15/0045; A61K 9/0078

(57) ABSTRACT

Systems and methods of delivering a drug product by inhalation can include a prodrug composition.

### PRIORTY CLAIM

This application claims priority to U.S. Provisional Patent Application No. 63/137,881, filed Jan. 15, 2021, which is incorporated by reference in its entirety.

### TECHNICAL FIELD

This invention relates to pharmaceutical compositions and methods of treatment by inhalation.

### BACKGROUND

Active ingredients, such as drugs or pharmaceuticals, can be delivered to patients in deliberate fashion using one or more prodrugs. Active ingredients can also be delivered to patients in combination with at least one other active or drug in the composition as part of a drug delivery system. In certain instances, the prodrugs themselves may have biological activity as well as the ability to convert or transform into one or more additional active drugs or active ingredients.

### SUMMARY

Prodrug design is an important part of drug discovery and can offer many advantages over parent drugs such as increased solubility, enhanced stability, improved bioavailability, reduced side effects, customization of pharmacokinetic profiles, improved organoleptics and better selectivity. The selection and design of the prodrug can be affected by the site of drug delivery, the tissue type, permeation, enzymatic conversion, steric hindrance, and other molecular considerations.

Delivery of drugs or pharmaceuticals transdermally, across lung tissue, or transmucosally can require that the prodrug, drug, active or pharmaceutical alone or in combination with a permeation enhancer and/or otherwise cross at least one biological membrane partially or completely in an effective and efficient manner.

In general, a method of treating a medical condition in a subject, such as a mammal or human subject can include administering by inhalation a composition including a prodrug, the prodrug passing through lung tissue to achieve an effective plasma concentration of a pharmaceutically active form of the prodrug in the human subject.

In another aspect, an inhalation device can include a housing and a composition within the housing, the composition including a prodrug. In certain embodiments, the method can include providing the composition in an inhaler.

The composition can be a liquid or a powder having a particle size of less than 1 micron. The composition can be a liquid or a powder having a particle size of less than 3 microns. The composition can be a liquid or a powder having a particle size of less than 5 microns. The composition can be a liquid or a powder having a particle size of less than 7 microns. The composition can be a liquid or a powder having a particle size of less than 10 microns. The composition can be a liquid or a powder having a particle size of less than 12 microns. The composition can be a liquid or a powder having a particle size of less than 15 microns. In certain embodiments, the inhaler can include a propellant.

In certain embodiments, the method can include providing the composition in a nebulizer.

In certain embodiments, the method can include providing the composition in an aerosol. In certain embodiments, providing the composition in an aerosol can include rapidly heating the composition to vaporize or sublimate the composition.

In certain embodiments, the composition can consist essentially of a prodrug.

In certain embodiments, the composition can consist essentially of the prodrug and epinephrine.

In certain embodiments, the composition can consist essentially of epinephrine and a prodrug of epinephrine.

In certain embodiments, composition can consist essentially of the prodrug and a second prodrug.

In certain embodiments, the prodrug is an ester of a pharmaceutically active compound.

In certain embodiments, the prodrug includes an alkyl ester of a pharmaceutically active compound.

In certain embodiments, the prodrug includes a butyl ester of a pharmaceutically active compound.

In certain embodiments, the prodrug includes an isopropyl ester pharmaceutically active compound.

In certain embodiments, the prodrug includes an ethyl ester pharmaceutically active compound.

In certain embodiments, the prodrug includes an ester of epinephrine.

certain embodiments, the prodrug is converted to an active compound.

certain embodiments, the medical condition is in a spectrum of anaphylaxis. In certain embodiments, the medical condition is an emergency or acute medical condition. In certain embodiments, a medical condition is a chronic medical condition. A medical condition can include an allergic reaction. In certain embodiments, the medical condition is a cardiac abnormality. In certain conditions, a medical condition can include urticaria and mast cell disorders, stress urinary incontinence. In certain embodiments, the medical condition is a pulmonary abnormality.

In certain embodiments, the composition including a prodrug can include more than one prodrug with each prodrug being a derivative of a pharmaceutically active ingredient. The composition including a prodrug may be a combination of different prodrugs where each prodrug is a derivative of a different pharmaceutical active ingredient. In some of these embodiments, one of the prodrugs can be dipivefrin.

In certain embodiments, a first prodrug is a first ester of epinephrine and a second prodrug is a second ester of epinephrine, the first ester of epinephrine and the second ester of epinephrine being different in chemical composition or constitution.

certain embodiments, the housing can be a blister-based housing. The composition can include a preloaded dose of a micronized API in an inhalable range.

In certain embodiments, housing can include a capsule comprising a unit dose of a powder of the composition.

In certain embodiments, the composition can include a prodrug and epinephrine.

In certain embodiments, the composition can include epinephrine and a prodrug of epinephrine.

In certain embodiments, the composition can include the prodrug and a second prodrug.

In general, a method of treating a medical condition in a mammal can include administering a therapeutically effective amount of a composition including a prodrug and epinephrine.

In certain embodiments, a method of treating a medical condition in a mammal can include administering a therapeutically effective amount of a composition including epinephrine and a prodrug of epinephrine.

In certain embodiments, the composition can be delivered both locally and systemically.

In general, a method of treating a medical condition in a mammal can include administering a therapeutically effective amount of a composition including a prodrug and a second prodrug and delivering the composition both locally and systemically.

In certain embodiments, the prodrug is a compound of formula (I), wherein

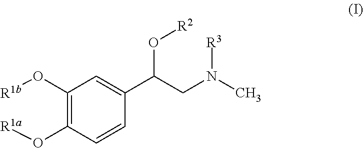

each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R^{1a}, R^{1b}, R² and R³ is not H, or a pharmaceutically acceptable salt thereof.

In certain embodiments, R² and R³ are H and each R^{1a} and R^{1b}, independently, can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, both of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, one of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl.

A prodrug can be structured to ensure its variable or customizable metabolic stability or protection, e.g., from enzymatic cleavage until a desired target is reached to alleviate certain side effects and/or enhance efficacy. Enzymatic cleavage can result from endogenous enzymes for example. In certain situations, enzymes can be intentionally added to a body to enhance metabolism for example.

In certain embodiments, the composition including a prodrug is administered in a dose of greater than 0.05 mg, greater than 0.1 mg, greater than 0.2 mg, greater than 0.3 mg, greater than 0.4 mg, greater than 0.5 mg, greater than 1.0 mg, greater than 2.0 mg, greater than 3.0 mg, greater than 4.0 mg, greater than 4.5 mg, less than 5.0 mg, less than 4.5 mg, less than 4.0 mg, less than 3.5 mg, less than 3.0 mg, less than 2.0 mg, and less than 1.0 mg.

In certain embodiments, the composition has a Cmax of greater than 5 mg/kg, greater than 10 mg/kg, greater than 20 mg/kg, greater than 25 mg/kg, greater than 50 mg/kg, greater than 100 mg/kg, greater than 200 mg/kg, greater than 250 mg/kg, less than 300 mg/kg, less than 250 mg/kg, less than 200 mg/kg, less than 150 mg/kg, less than 100 mg/kg, less than 50 mg/kg, less than 25 mg/kg or less than 20 mg/kg.

In certain embodiments, the effective plasma concentration of a pharmaceutically active form of the prodrug has a Tmax of greater than 0.5 seconds, greater than 1 second, greater than 5 seconds, greater than 10 seconds, greater than 20 seconds, greater than 30 seconds, less than 40 seconds, less than 30 seconds, less than 20 seconds, less than 10 seconds, less than 5 seconds, and less than 1 second.

Other aspects, embodiments, and features will be apparent from the following description, the drawings, and the claims.

### BRIEF DESCRIPTION OF THE DRAWINGS

FIG. 1 depicts an inhaler.

FIGS. 2A-2B depict stability tests in BAL fluid.

FIGS. 3A-3U depict in vitro permeability tests and preclinical pharmacokinetic data

FIG. 4A-4B depicts chromatograms of the AQEP-09 prodrug and Dipivefrin with lactose monohydrate.

FIGS. 5 and 6 depict the particle size distribution for a micronized powder of Dipivefrin measured by aerodynamic particle sizer.

FIGS. 7 and 8 depict the particle size distribution for a micronized powder of Diisobutyryl L-epinephrine (AQEP-09) measured by aerodynamic particle sizer.

### DETAILED DESCRIPTION

Prodrugs can provide enhanced delivery of an active pharmaceutical ingredient, such as epinephrine, for example. Tissue surfaces, such as lung tissue, can be a route for delivering drugs to the body due to the fact that the tissue is highly vascularized and permeable, providing increased bioavailability and rapid onset of action because it does not pass through the digestive system and thereby avoids first pass metabolism. Prodrugs are described in International Patent Application Publication No. 2021/087359 A1, which is incorporated by reference in its entirety.

A pharmaceutical composition can be designed to deliver a prodrug for a pharmaceutically active component via inhalation in a deliberate and tailored way. Delivery of certain active compounds, such as epinephrine, is characterized by certain unique challenges. The compound is hydrophilic, endogenous, highly variable, requires rapid delivery, and promotes vasoconstriction. Thus, the concentration and timing of its delivery is often critical to manage and not easily accomplished. An effective approach to delivering epinephrine can be with a system that allows the compound to penetrate lung tissue, particularly the ciliated apical surface, mucociliary epithelium and microporous membrane.

A pharmaceutically active compound can be applied to lung tissue most readily by inhalation. There are a number of devices for the inhalation delivery of drugs, including dry powder inhalers (DPI), atomizers, aerosolizers, nebulizers, and pressurized metered dose inhalers. The aerosols or drug vaporizers produced by the devices can contain an excipient. In certain embodiments, a method can produce aerosols in the absence of excipient. Examples of devices suitable for drug inhalation can be found in U.S. Pat. No. 7,766,013, U.S. Pat. No. 9,107,832, U.S. Pat. No. 10,004,858, U.S. Pat. No. 9,192,675 and U.S. Patent Publication No. 2008/0078382, each of which is incorporated by reference in its entirety.

A method and device are provided to deliver a pharmaceutically active compound to lung tissue. The method and device can deliver a powder, mist, or aerosol to the lung of a human subject. The powder, mist, or aerosol can have a desired mass mean aerodynamic diameter (MMAD) , i.e., from molecular to about 10 microns, which can be used to effectively deliver a physiologically active compound to organs and tissues such as the lung, eye, mucosa and skin. The MMAD can be less than 10 microns, less than 5 microns, less than 4 microns, less than 3 microns, less than 2 microns, less than 1 micron, less than 500 nanometers, less than 250 nanometer, less than 100 nanometers, or less than 50 nanometers. The particle size can be between 5 nm and 100 nm. For example, an aerosol can be formed through vaporization of the compound while mixing the resulting vapor with a gas, in a ratio to form the desired particle size when a stable concentration of particles in the gas is reached. In general, the method and device can volatilize a pharmaceutically active compound and administering the volatilized compound in the form of an aerosol to a patient. The ratio of mass of vaporized compound to the volume of the mixing gas can be adjusted to alter the particle size distribution.

Without being limited to a specific method, the method can include volatilizing or sublimating the compound by rapidly heating a composition. For example, the composition can be coated or otherwise deposited on a substrate as a layer that is less than 10 microns thick. Preferably, the layer can be less than 5 microns, 4 microns, 3 microns, 2 microns, 1 microns, 500 nanometers, 250 nanometers, 100 nanometers, or 50 nanometers. The layer can be discontinuous and variable in thickness. In other embodiments, the layer can be substantially uniform in thickness. The substrate can be heated at a high rate to create a vapor. The composition can be vaporized by applying an alternating magnetic field or current to a foil substrate to rapidly heat (by Joule heating or resistance heating) the compound is vaporized sequentially over no more than about a one second period of time. Such heating rate can be greater than 2,000° C./s, 5,000° C./s, 7,500° C./s, or 10,000° C./s. The heating can take place in less than 0.1 seconds, less than 0.2 seconds, less than 0.5 seconds, or less than 0.8 seconds. The substrate can be a metal foil, for example, aluminum, platinum, palladium, or a stainless steel.

The composition can be deposited on the substrate by any acceptable method, including but not limited to, spray coating, dip coating, spin coating or melt coating a material including the prodrug onto the substrate. A solvent can be used to deposit the composition on the substrate. The solvent can be removed to provide a dry material on the substrate.

The vapor can be swept to the lung by a carrier gas. The carrier gas can be provided by an external source, such as a propellant, or by inhalation, or combinations thereof.

The aerosols of the various embodiments are typically formed by preparing a composition containing a drug composition on a heat-conductive and impermeable substrate and heating said substrate to vaporize the composition and cooling the vapor thereby producing aerosol particles containing said drug composition. A layer is generally a thin coating that can be used efficiently as a drug release platform. Desirable features for a layer include having sufficient drug loading capacity, acceptable formulation stability, and also being non-toxic, low or tolerable toxicity, biocompatible and biodegradable. Rapid heating in combination with the gas flow helps reduce the amount of decomposition. Thus, a heat source can heat the substrate to a temperature of greater than 50° C., preferably at least 100° C., preferably at least 150° C., preferably at least 200° C., preferably at least 250° C., more preferably at least 300° C. or more preferably at least 350° C. to produce substantially complete volatilization of the drug composition from the substrate within a period of 2 seconds, preferably, within 1 second, and more preferably, within 0.5 seconds. A gas flow rate over the vaporizing compound of between about 4 and 50 L/minute can sweep the prodrug into the lungs.

In certain circumstances, 0.25 mg, 0.5 mg, 0.75 mg, or 1 mg of the composition can be vaporized in less than 100 milliseconds from the start of heating. More preferably, the same amount of composition described above is vaporized in less than 75 milliseconds, 50 milliseconds, 25 milliseconds, or 10 milliseconds from the start of heating. This vaporization can be generated from a 1 micron, 2 micron, 3 micron, 4 micron or 5 micron particle on a substrate.

The inhalant can be a solution, suspension of liquid or solid particles of a substance (or substances) in a gas. The inhalant can be greater than 10 percent by weight of the drug. Preferably, the inhalant can be greater than 20 percent by weight of the drug. More preferably, the inhalant can be greater than 30 percent, 40 percent, 50 percent, 60 percent, 70 percent, 80 percent, 90 percent, 95 percent, 97 percent, 98 percent, or 99 percent by weight of the drug. In certain circumstances, the inhalant can be 100 percent by weight of the drug.

The inhalant can contain less than 10 percent by weight of drug decomposition products. Preferably, the inhalant can contain less than 5 percent by weight of drug decomposition products. More preferably, the inhalant can contain less than 3 percent, 2 percent, 1 percent, 0.5 percent, 0.25 percent or 0.1 percent by weight of drug decomposition products.

The vaporized or sublimated compound particle size can be influenced by the density and other physicochemical properties (for example, solubility, surface activity, complexation, dissociation constant, partition coefficient, isomerism) of the prodrug compound, the polarity of the compound, and temperature. In certain circumstances, the compound can be a salt. The hydrophilic or hydrophobic nature of the compound can affect the eventual particle size. The prodrug can be combined in a sublimable carrier such as an alcohol or an aromatic, for example, menthol, thymol, camphor, t-butanol, trichloro-t-butanol, imidazole, coumarin, acetic acid (glacial), dimethylsulfone, urea, vanillin, camphene, salicylamide, or 2-aminopyridine.

Another suitable inhaler can be a dry powder inhaler or DPI. The compound can be formulated in a dry powder containing an active pharmaceutical ingredient (API) with or without other adjuvants, which can be milled to a desirable particle range. The desired particle range influences the absorption and/or pharmacodynamic effects of the API. An inhalable range refers to particles with an aerodynamic diameter range of 0.5-5 μm. Particles in the range 0.5-3 μm are appropriate for systemic absorption in the distal region including alveoli, and 3-5 μm for local action in the terminal bronchioles. Preferably, the dry powder particles should be monodispersed and spherical or near spherical in shape. An excipient such as lactose (or other additional excipients) can be used to increase fine particle dose and flowability. A DPI can be designed to have a de-aggregation engine to deaggregate the API from its coarser particle form. A DPI can operate in an active/passive manner, with the energy to de-agglomerate powder provided by the device or the patient. The DPI is dependent on turbulence in the device and the patient's inspiratory flow rate. The DPI can provide differing airflow resistance depending on its design. A low resistance requires larger effort for the patient (deep inhalation at high flow rate) and a high particle velocity may cause higher throat deposition. A high resistance takes little effort but will also feel more constricted due to low flow. An intermediate resistance is desirable.

A micronized API can be a micronized drug in an inhalable range, about 0.5-5 μm in aerodynamic diameter. Micronization is a process for reducing the diameter of a solid material's particles to enable the solubility of the API. The traditional techniques for micronization have been based on friction to reduce the particle size, typically accomplished by milling or grinding particles. A micronized drug and its carrier, e.g. lactose, are de-aggregated by a patient's inhalation through the device. Particles may also made by processes other than micronization. For example, uniform particles of identical size and shape could be produced by Particle Replication in Nonwetting Templates (PRINT) Technlology.

The API can include a prodrug as described herein. In certain examples, a dose can be provided in a 10% API/lactose blend. The blend can include lactose or maltose. Following milling the compound can be mixed with a carrier or excipient to impart flowability. Other methods of providing powder include high pressure homogenization, spray drying, lyophilization of solutions in water-organic solvent mixtures, and lyophilization of solutions inorganic solvents. Additional examples of adjuvants include inorganic salts, Fumaryl diketopiperazine, saccharides, D-mannitol, sorbitol, erythritol, D-Raffinose, glucose, Trehalose, cyclodextrin, or magnesium stearate. The saccharides can be anhydrous or hydrates. A formulation, for example for a dry powder inhaler (DPI), can include a single carrier or a mixture of carriers. In some formulations, magnesium stearate can be used as a particle stabilizer along with a carrier such as lactose or maltose. Other excipients that can be used in the formulation include albumin and dipalmitoylphosphatidylcholine (DPPC) which can be used to enhance the aerosolization process.

In certain circumstances, a dose can be between 0.1-5 mg, for example a 0.3 mg dose, a 0.7 mg dose, a 0.25 mg dose, a 0.6 mg dose. A dose can be a target 4.8 mg target dose. A dose can be administered in a blister-based or in a capsule-based device. The composition can be contained in a blister or capsule that can be loaded into a housing of an inhaler. In certain examples, a delivered dose can include 70% of the original dose pre-loaded in a blister-based or capsule-based device. In certain examples, a 10% API/lactose blend can be provided such that 4.8 mg is pre-filled in a capsule or blister-based device.

Another suitable inhaler can be a nebulizer, which generates an aerosol from a liquid. For example, the nebulizer can breakup a liquid jet or by ultrasonic vibration of the liquid with or without a nozzle. A jet nebulizer can draw up liquid by capillary action such that the liquid reaches a jet stream, is drawn into the jet stream, and is shattered into small particles. An ultrasonic nebulizer can use electric current to produce sound waves that break up liquid into an aerosol. An ultrasonic nebulizer can include a ceramic transducer (including piezo electronic technology) that changes electrical energy into pressure energy. The transducer vibrates at a very high frequency of up to about 1.5 mHz. The vibrational energy is transmitted through liquid and focused on a flexible diaphragm that vibrates. The diaphragm is in contact with the liquid to be aerosolized and shakes the solution into particles, generating a fine mist at high frequencies. Ultrasonic nebulizers may produce a more consistent particle size than do jet nebulizers and may produce very large volumes of respirable particles with much greater deposition into the lungs. Liquid formulations can be prepared and stored under aseptic or sterile conditions since they can harbor microorganisms. This can necessitate the use of preservatives or unit dose packaging. Additionally, solvents, detergents and other agents can be used to stabilize the drug formulation.

Another suitable inhaler can be a pressurized metered dose inhaler. Such an inhaler can package the compound in a canister under pressure with a solvent and propellant mixture, usually chlorofluorocarbons (CFC's, which are being phased out due to environmental concerns), or hydrofluoroalkanes (HFA's). Upon being dispensed a jet of the mixture is ejected through a valve and nozzle and the propellant “flashes off” leaving an aerosol of the compound. One challenge with this type of inhaler is variability in a portion of the dose that can be lost on the walls of the actuator, and due to the high speed ejection of the aerosol from the nozzle, some of the dose can impact ballistically on the tongue, mouth and throat and may not arrive at the lung tissue.

In certain circumstances, the drug of the composition can be epinephrine or an epinephrine derivative, for example a prodrug of epinephrine.

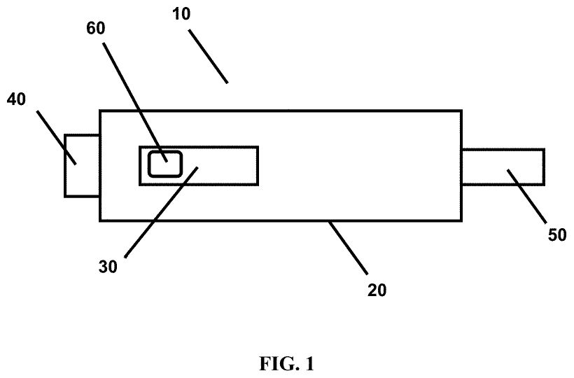

Referring to FIG. 1, inhaler 10 can have a housing 20. The housing 20 contains a composition 60. The housing 20 includes gas source 40, which can be an opening to allow for air flow or a pressurized gas containing a carrier gas. The housing 20 includes a dosing orifice 50 from which the vaporized, nebulized, or powdered prodrug is delivered by inhalation. The subject can inhale the material from dosing orifice 50. Composition 60 can be contained in a carrier 30. Carrier 30 can be a blister or capsule containing the composition.

As described above, carrier 30 can be a vaporization unit including the prodrug deposited onto a substrate that is heated to rapidly to vaporize the prodrug. In another implementation, carrier 30 can be a liquid reservoir that nebulizes the prodrug. In another implementation, carrier 30 can be a powder source including the prodrug.

As described above, composition 30 can be a vaporization unit including the prodrug deposited onto a substrate that is heated to rapidly to vaporize the prodrug. In another implementation, composition 30 can be a liquid reservoir that nebulizes the prodrug. In another implementation, composition 30 can be a powder source including the prodrug.

The device can provide gas flow to carry the vaporized prodrug into the lung of the subject. The gas flow from gas source 40 can be air or oxygen. The flow can be induced by inhalation by the subject. Alternatively, the flow can be provided from an external source. In another embodiment, gas flow from gas source 40 and be a pressurized carrier gas such as helium or a fluorinated hydrocarbon.

Dosing orifice 50 can be inserted directly into the mouth of the subject for inhalation. In certain embodiments, dosing orifice 50 can be connected to a face mask that covers the mouth of the subject.

The prodrugs described herein can be heated to temperatures preferably in the range of 50° C. to 400° C. without significant thermal degradation. In certain embodiments, the prodrugs can be subjected to rapid heating. In certain examples, the prodrug can be heated to temperatures in the range of about 100° C. to 500° C. without significant thermal degradation. For example, a prodrug can be heated to about 50° C., about 75° C., about 100° C., about 125° C., about 150° C., about 200° C., about 250° C., about 300° C., about 350° C., about 400° C., about 450° C., or about 500° C. . The high thermal stability of prodrug can facilitate aerosolization of a carrier-free drug formulation without decreasing the purity of the aerosol, and, without using a carrier, a substantially pure aerosol may be formed. Such an approach can generate solid aerosols as well as liquid aerosols.

In a given dose, for example between 1-10 mg, a powder containing an API, can be delivered with or without the use of excipients. In other examples, the dose could be between 0.1-20 mg. The API can be epinephirine, a prodrug, such as a prodrug of epinephrine, a combination of epinephrine and a prodrug of epinephrine, or a combination of different prodrugs of epinephrine.

The dose can be delivered through API filled capsules. The capsules can be filled with a powder including the API. The powder can be a micronized powder containing the API only. In other examples, the powder can be a micronized API and lactose blend for example. In yet other formulations, the powder can be a formulation of API, for example an engineered or spray-dried formulation of API, and excipients.

A prodrug design can provide an alternative for the delivery of epinephrine, and indeed, for other active pharmaceutical ingredients. A prodrug can present improved hydrophobicity, better permeation, dose reduction, and enhanced speed of absorption. It can also provide alternative compositions with unique stability profiles. For example, while epinephrine is stabilized by sodium metabisulfite, the prodrug dipivefrin was found to be unstable in sodium metabisulfite. Other prodrugs could have similar stability and/or be designed based on the desired stability profile exhibited with certain additives. A prodrug that is not absorbed in the stomach can also avoid, minimize or eliminate the side effect of epigastric pain. Moreover, a prodrug can result in reduced adrenergic receptor binding, resulting in reduced variability of vasoconstriction and more stability. The epinephrine prodrug can require conversion in the blood, which can cause a delay in epinephrine exposure as a function of its conversion rate, and since the molecular weight is often higher than that of the active pharmaceutical ingredient, it can also require a higher mass of drug loading (e.g. if the prodrug is twice the molecular weight of the active pharmaceutical ingredient, it can require twice the drug loading).

A prodrug can be metabolized, for example by hydrolysis. Metabolism can occur through enzymatic conversion, for example through exogenous or endogenous hydrolytic enzymes, which convert a prodrug into an active compound. A prodrug can be converted at various times and in various ways in the body. A prodrug can be designed based on a targeted approach for in any suitable manner based on where and when conversion is desired. In some instances, prodrug conversion can occur locally, for example, within the lung. In some instances, prodrug conversion can occur systemically (e.g. in circulation). In some situations, prodrug conversion occurs intracellularly (e.g., antiviral nucleoside analogs, lipid-lowering statins). In some situations, prodrug conversion can occur extracellularly, for examples in digestive fluids or other extracellular body fluids). In some instances, concomitant administration of components that enable conversion of the prodrug to the active parent. A prodrug may contain a hydrolysis inhibitor (before supplying the drug), and an accelerator when supplying the drug. Each prodrug or partial hydrolysis product can have its own pharmacological activity

certain embodiments, at least half of the administered prodrug is converted in less than 240 minutes. In certain embodiments, at least half of the administered prodrug is converted in less than 120 minutes. In other embodiments, at least half of the administered prodrug is converted in less than 60 minutes. In other embodiments, at least half of the administered prodrug is converted in less than 30 minutes. In other embodiments, at least half of the administered prodrug is converted in less than 15 minutes. In other embodiments, at least half of the administered prodrug is converted in less than 10 minutes. In other embodiments, at least half of the administered prodrug is converted in less than 5 minutes. In other embodiments, at least half of the administered prodrug is converted in less than 1 minute. In other embodiments, at least half of the administered prodrug is converted in less than 30 seconds. In other embodiments, at least half of the administered prodrug is converted in less than 15 seconds. In other embodiments, at least half of the administered prodrug is converted in less than 10 seconds. In other embodiments, at least half of the administered prodrug is converted in less than 5 seconds.

In certain embodiments, a prodrug can be designed to convert to produce a concentration of active compound of between 5 pg/ml to about 40 ng/ml in a period of less than 120 minutes. The prodrug can be designed to convert to produce a concentration of active compound of between 20 pg/ml to about 40 ng/ml in a period of less than 60 minutes. A prodrug can be designed to convert to produce a concentration of active compound of between 20 pg/ml to about 40 ng/ml in a period of less than 30 minutes. The prodrug can be designed to convert to produce a concentration of active compound of between 20 pg/ml to about 40 ng/ml in a period of less than 15 minutes. The prodrug can be designed to convert to produce a concentration of active compound of between 20 pg/ml to about 40 ng/ml in a period of less than 10 minutes. The prodrug can be designed to convert to produce a concentration of active compound of between 20 pg/ml to about 40 ng/ml in a period of less than 5 minutes. The prodrug can be designed to convert to produce a concentration of active compound of between 20 pg/ml to about 40 ng/ml in a period of less than 1 minute.

Other prodrugs for the delivery of an active pharmaceutical compound have been explored and are described herein. For example, the prodrug can be a compound of formula (I)

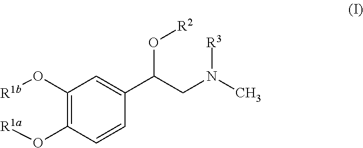

or a pharmaceutically acceptable salt thereof.

formula I, each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R^{1a}, R^{1b}, R² and R³ is not H, or a pharmaceutically acceptable salt thereof. In preferred circumstances, R² and R³ are H and each R^{1a} and R^{1b}, independently, can be C1-C16 acyl, for example, ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In certain circumstances, each of R^{1a} and R^{1b} is the same and is not H and R² and R³ are H. In certain circumstances, both of R^{1a} and R^{1b} are not pivaloyl.

The compound can be in a free-base form. Alternatively, the compound of formula I can be a pharmaceutically acceptable salt. The pharmaceutically acceptable salt can be an acid addition salt or a base addition salt. Acid addition salts can be prepared by reacting the purified compound in its free-based form with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid. Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium, alkylammonium, substituted alkylammonium and N⁺(C₁₋₄alkyl)₄ salts. The alkyl can be a hydroxyalkyl. Other pharmaceutically acceptable salts of the compound can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

To deliver epinephrine, a class of prodrug compounds can have modifications made to the R^{1a}, R^{1b}, R² and R³ groups of epinephrine as shown below. The R^{1a} and R^{1b} groups can include esters, amides, carbonates and carbamates, orthoesters or acetals. The groups can include for example, alkyl esters, chloroalkyl esters, amides, alkyl amides, chloroalkyl amides. The R² groups can include benzylic alcohol modification. The R³ group can include amine modification or oxazolidines. An ideal prodrug would have one or more of the following attributes, is biologically acceptable, penetrates one or more membrane, is stable and converts in the body, tissue or blood. In some cases, the prodrug may not need any permeation enhancers at all but rather permeate sufficiently by itself. The conversion of the prodrug to active is not predictable based on chain length of the R^{1a}, R^{1b}, R² and R³ groups. In particular, a tertiary group at the second atom of the R^{1a}, R^{1b}, R² or R³ group. The permeation of the prodrug is also unpredictable based on the R^{1a}, R^{1b}, R² and R³ groups.

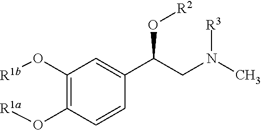

The prodrug selection process for an active pharmaceutical ingredient was conducted by first synthesizing prodrugs with various substituents, conducting ex vivo permeation studies, and following those with in vitro hydrolysis assay using a biological fluid (e.g., human whole blood).

### Synthesis

General synthetic procedures were used to synthesize epinephrine prodrugs as shown below.

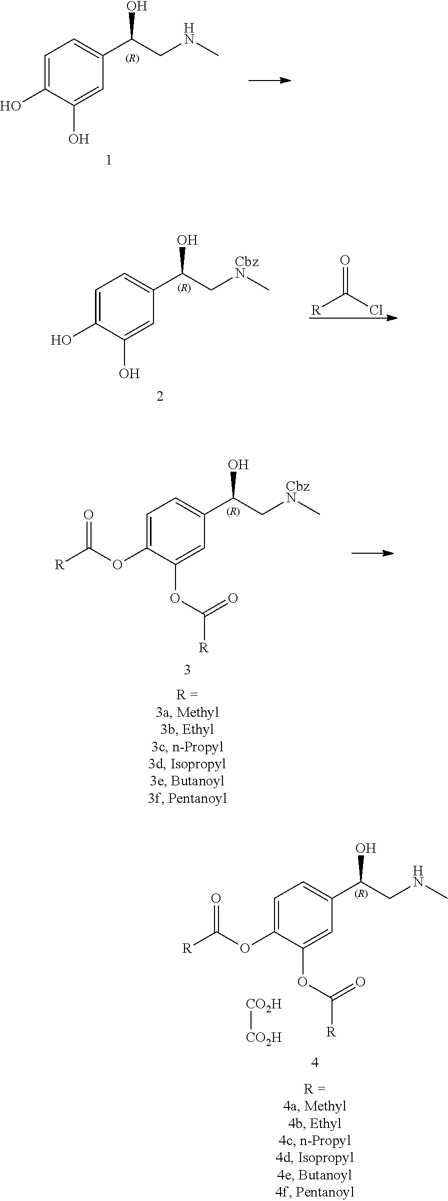

### Preparation of 2:

To a mixture of (−)-epinephrine (5 g) in water (50 ml) and THF (25 ml), was added NaHCO₃ (4.6 g, 2 equiv) and stirred for 5 min at 0-5 ° C., then added a solution of N-(Benzyloxycarbonyloxy)succinimide (6.82 g, 1 equiv) in THF (25 ml) slowly and stirred at RT for 12 h. Solvent was removed in rotary evaporator, the slid residue was extracted with ethyl acetate (100 mL), washed with 2N HCl (50 mL), followed by brine (100 mL), dried over anhydrous sodium sulfate and the solvent was removed to obtain compound 2 (8.5 g) as a thick brown syrup.

General Procedure for Preparation of Esters 3a-3f.

To a solution of compound 2 (1 equiv), triethylamine (3 equiv) in dichloromethane (30 volumes to compound 2) was added the corresponding acid chloride (1.8 equiv) dropwise at 0-5° C. Then, the mixture was gradually warmed to room temperature and stirred for 12 h. The reaction mixture was quenched with saturated NaHCO₃ solution (30 volumes to compound 2). The organic phase was separated, dried over anhydrous sodium sulfate and concentrated to residue which was purified by column chromatography to isolate clean 3a-3f (˜60% yield) as oils. Monosubstituted epinephrine prodrugs (not shown in the scheme above) were synthesised using lower equivalent of acid chloride.

General Procedure for Preparation of 4a-4f.

A mixture of 3a-3f in methanol (˜20 vol), oxalic acid (1 equiv), 10% Pd/C (50% wet, 20% wt to the starting material) was stirred under hydrogen atmosphere (using a balloon) for 12 h; TLC was used to ensure completion of the reaction. The catalyst was filtered through celite bed and the filtrate was concentrated to dryness. The solids were suspended in Methyl tert-butyl ether (5 vol), stirred for 30 min; filtered the solids and dried. 4a-4f were isolated as white solids and confirmed by NMR and Mass spec.

### Synthesis of Biscarbonate 6:

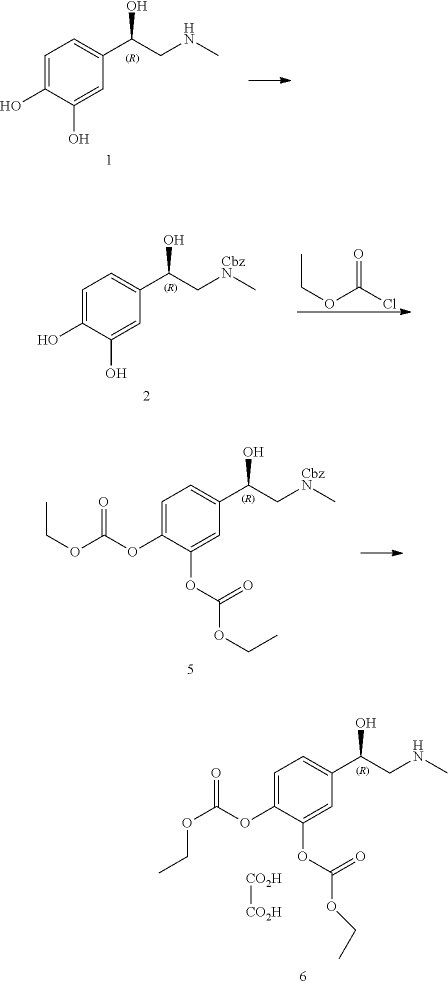

### Compound 5:

Same procedures as 3a-3f was followed.

### Compound 6:

Same procedures as 4a-4f was followed.

Alternative synthetic route for AQEP-09

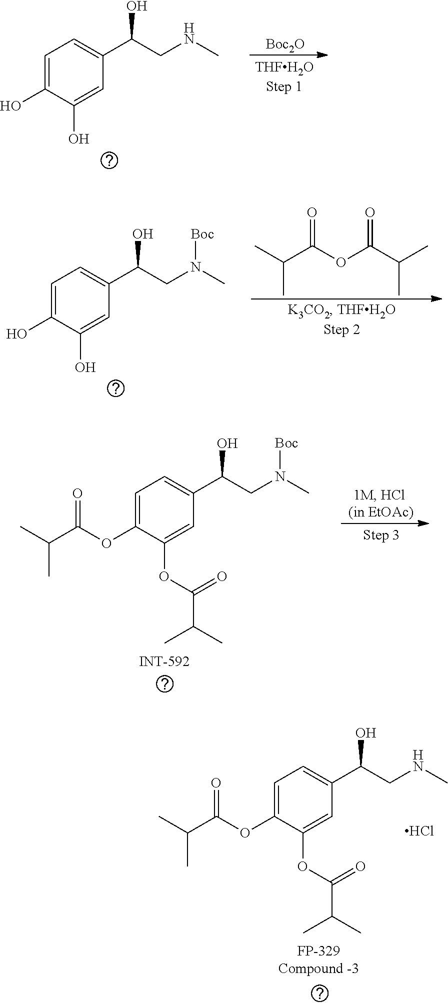

An alternate synthetic method was used to manufacture AQEP-09. In this method, epinephrine was reacted with Boc anhydride in a mixture of tetrahydrofuran and water at room temperature until reaction is complete. The intermediate N-Boc-epinephrine was diluted with tetrahydrofuran and potassium carbonate solution in water and reacted with isobutyric anhydride until acetylation of the two phenolic hydroxyl groups was complete. The product was then isolated with MTBE extraction followed by evaporation. The Boc deprotection was achieved using 1M hydrochloric acid in ethyl acetate after diluting the intermediate with n-heptane. The reaction mixture initially becomes clear after the addition of HCl in ethyl acetate. Upon completion of reaction, the product precipitates out which was collected and purified.

Exemplary prodrugs are provided in the table below, which were synthesized by similar procedures.

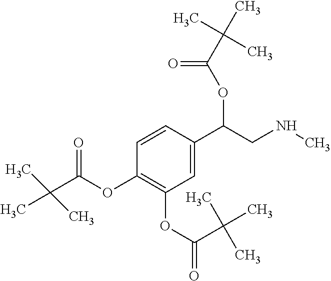 [SMILES: CNCC(OC(=O)C(C)(C)C)c1ccc(OC(=O)C(C)(C)C)c(OC(=O)C(C)(C)C)c1]

AQEP-01 Tripivaloyl ester

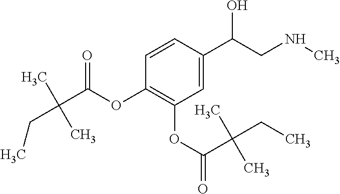 [SMILES: CCC(C)(C)C(=O)Oc1ccc(C(O)CNC)cc1OC(=O)C(C)(C)CC]

AQEP-02 Methyl dipi ester

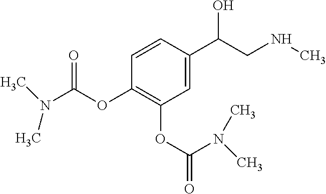

AQEP-03 dimethylamino

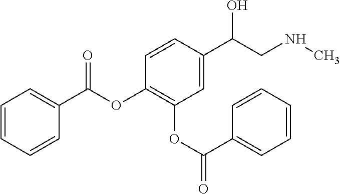 [SMILES: CNCC(O)c1ccc(OC(=O)c2ccccc2)c(OC(=O)c2ccccc2)c1]

AQEP-04 Dibenzoyl ester

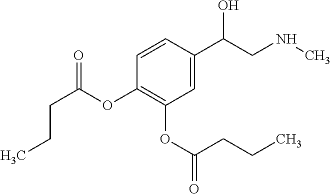 [SMILES: CCCC(=O)Oc1ccc(C(O)CNC)cc1OC(=O)CCC]

AQEP-05 Dipropyl ester

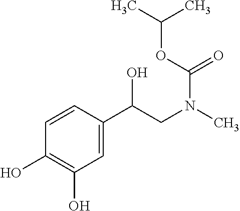 [SMILES: CC(C)OC(=O)N(C)CC(O)c1ccc(O)c(O)c1]

AQEP-06 Isopropyl carbamate

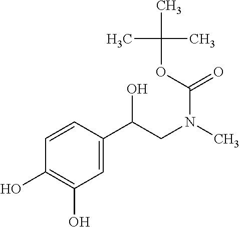 [SMILES: CN(CC(O)c1ccc(O)c(O)c1)C(=O)OC(C)(C)C]

AQEP-07 t-butyl carbamate

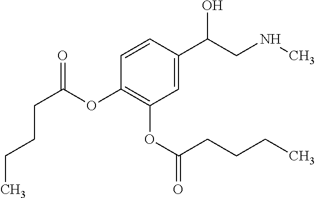 [SMILES: CCCCC(=O)Oc1ccc(C(O)CNC)cc1OC(=O)CCCC]

AQEP-08 Dibutyl ester

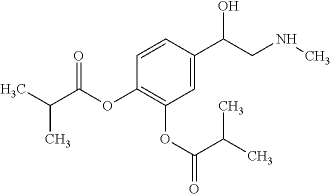 [SMILES: CNCC(O)c1ccc(OC(=O)C(C)C)c(OC(=O)C(C)C)c1]

AQEP-09 Diisopropyl ester

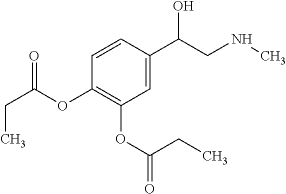 [SMILES: CCC(=O)Oc1ccc(C(O)CNC)cc1OC(=O)CC]

AQEP-10 Diethyl ester

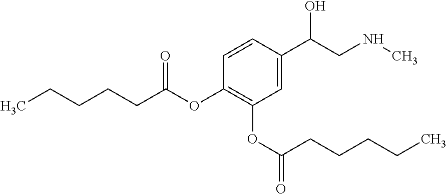 [SMILES: CCCCCC(=O)Oc1ccc(C(O)CNC)cc1OC(=O)CCCCC]

AQEP-11 Dipentyl ester

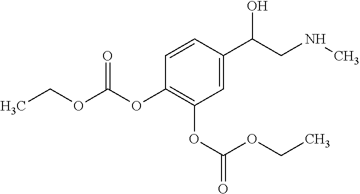 [SMILES: CCOC(=O)Oc1ccc(C(O)CNC)cc1OC(=O)OCC]

AQEP-12 Diethyl carbonate

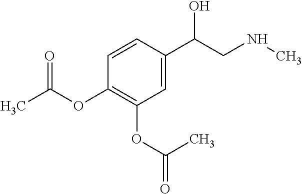 [SMILES: CNCC(O)c1ccc(OC(C)=O)c(OC(C)=O)c1]

AQEP-13 Dimethyl ester

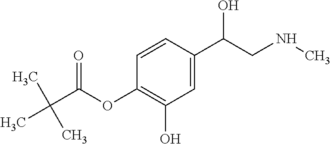 [SMILES: CNCC(O)c1ccc(OC(=O)C(C)(C)C)c(O)c1]

4-Pivaloylepinephrine

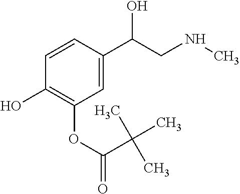 [SMILES: CNCC(O)c1ccc(O)c(OC(=O)C(C)(C)C)c1]

3-Pivaloylepinephrine
AQEP-14 Monopivaloyl ester oxalate

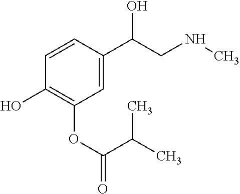 [SMILES: CNCC(O)c1ccc(O)c(OC(=O)C(C)C)c1]

3-isobutyryl epinephrine

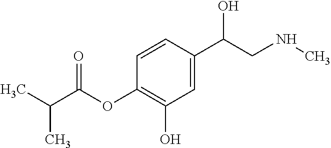 [SMILES: CNCC(O)c1ccc(OC(=O)C(C)C)c(O)c1]

4-isobutryryl epinephrine
AQEP-15 Monoisobutyryl epinephrine

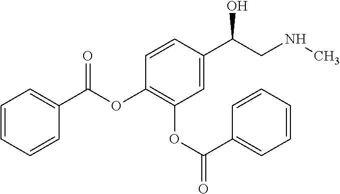 [SMILES: CNC[C@H](O)c1ccc(OC(=O)c2ccccc2)c(OC(=O)c2ccccc2)c1]

AQEP-16 Dibenzoyl ester HCl

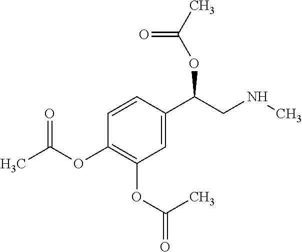 [SMILES: CNC[C@H](OC(C)=O)c1ccc(OC(C)=O)c(OC(C)=O)c1]

AQEP-17 triacetate

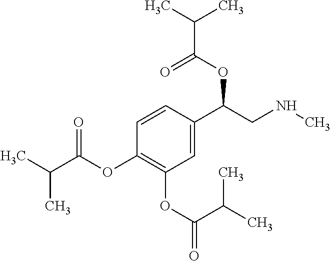 [SMILES: CNC[C@H](OC(=O)C(C)C)c1ccc(OC(=O)C(C)C)c(OC(=O)C(C)C)c1]

AQEP-18 Triisobutyryl ester

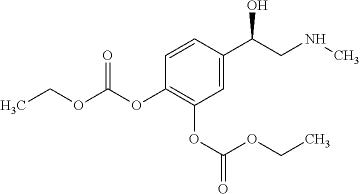 [SMILES: CCOC(=O)Oc1ccc([C@@H](O)CNC)cc1OC(=O)OCC]

AQEP-19 Diethyl carbonate

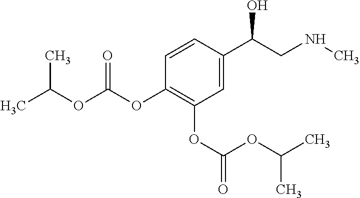 [SMILES: CNC[C@H](O)c1ccc(OC(=O)OC(C)C)c(OC(=O)OC(C)C)c1]

AQEP-20 Diisopropyl carbonate

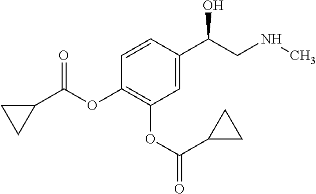 [SMILES: CNC[C@H](O)c1ccc(OC(=O)C2CC2)c(OC(=O)C2CC2)c1]

AQEP-21 Dicyclopropyl ester

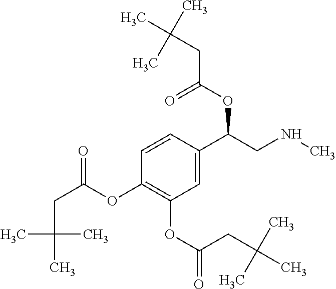 [SMILES: CNC[C@H](OC(=O)CC(C)(C)C)c1ccc(OC(=O)CC(C)(C)C)c(OC(=O)CC(C)(C)C)c1]

AQEP-22 (R)-4-(1-((3,3-dimethylbutanoyl)oxy)-
2-(methylamino)ethyl)-1,2-phenylene bis(3,3-
dimethylbutanoate) hydrochloride

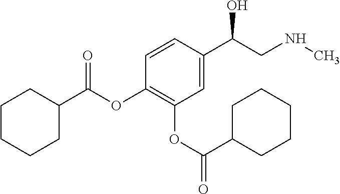 [SMILES: CNC[C@H](O)c1ccc(OC(=O)C2CCCCC2)c(OC(=O)C2CCCCC2)c1]

AQEP-23 Dicyclohexl ester

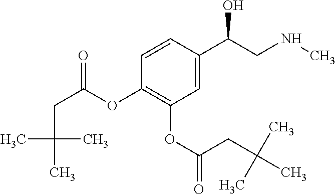 [SMILES: CNC[C@H](O)c1ccc(OC(=O)CC(C)(C)C)c(OC(=O)CC(C)(C)C)c1]

AQEP-24 (R)-4-(1-hydroxy-2-
(methylamino)ethyl)-1,2-phenylene bis(3,3-
dimethylbutanoate) hydrochloride

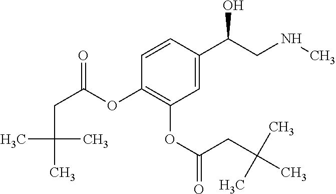 [SMILES: CNC[C@H](O)c1ccc(OC(=O)CC(C)(C)C)c(OC(=O)CC(C)(C)C)c1]

AQEP-24 (R)-4-(1-hydroxy-2-
(methylamino)ethyl)-1,2-phenylene bis(3,3-
dimethylbutanoate) hydrochloride

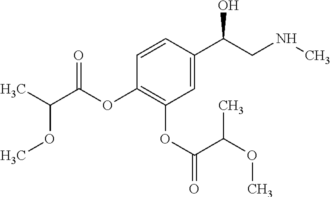 [SMILES: CNC[C@H](O)c1ccc(OC(=O)C(C)OC)c(OC(=O)C(C)OC)c1]

AQEP-25 4-((R)-1-hydroxy-2-
(methylamino)ethyl)-1,2-phenylene bis(2-
methoxypropanoate) hydrochloride

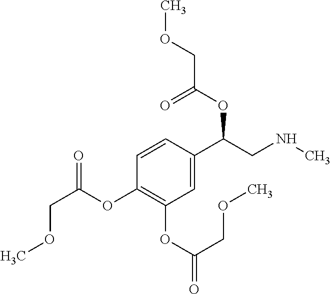

AQEP-26 (R)-4-(1-(2-methoxyacetoxy)-2-
(methylamino)ethyl)-1,2-phenylene bis(2-
methoxyacetate) hydrochloride

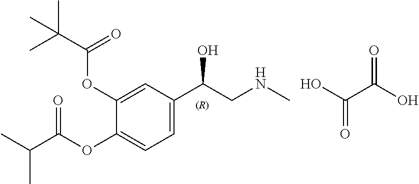

AQEP-27 (R)-5-(1-Hydroxy-2-
(methylamino)ethyl)-2-(isobutyryloxy)phenyl
pivalate oxalate

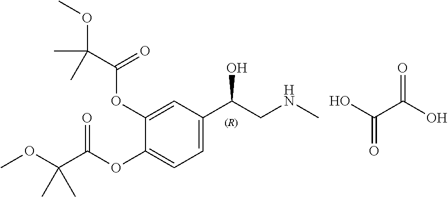

AQEP-28 (R)-4-(1-Hydroxy-2-
(methylamino)ethyl)-1,2-phenylene bis(2-
methoxy-2-methylpropanoate)oxalate

The monoesters AQEP-14 and AQEP-15 are mixtures of the two regioisomers.

The prodrug can be designed to be any particle size that enables it to be delivered effectively. In some embodiments, the prodrug has particle size of no more than 200 microns. In some embodiments, the prodrug has particle size of no more than 300 microns, the prodrug has particle size of no more than 400 microns.

The prodrug can be designed in a manner that permits effective metabolism or hydrolysis into an active compound. For example, in certain embodiments, the prodrug is an ester of a pharmaceutically active form of the prodrug. In certain embodiments, the prodrug includes an alkyl ester of a pharmaceutically active form of the prodrug. In certain embodiments, the prodrug includes a butyl ester of a pharmaceutically active form of the prodrug. In certain embodiments, the prodrug includes an isopropyl ester of a pharmaceutically active form of the prodrug. In certain embodiments, the prodrug includes an ethyl ester of a pharmaceutically active form of the prodrug. In certain embodiments, the prodrug includes an amide of a pharmaceutically active form of the prodrug. In certain embodiments, the prodrug includes a carbonate of a pharmaceutically active form of the prodrug.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be a pharmaceutically acceptable salt of a drug, a prodrug, a derivative, a drug complex or analog of a drug.

The term “prodrug” refers to a biologically inactive compound that can be metabolized in the body to produce a biologically active drug or the “prodrug” can be a biologically active compound where in addition to its inherent biological activity can be metabolized to another or even preferred biologically active drug. In certain embodiments, the prodrug can have its own biological activity that can be similar to or different from the active drug. For example, the prodrug can be an ester of epinephrine, for example, dipivefrin which is hydrolysed into epinephrine. See, e.g., J. Anderson, et al., Site of ocular hydrolysis of a prodrug, dipivefrin, and a comparison of its ocular metabolism with that of the parent compounds, epinephrine, Invest., Ophthalmol. Vis. Sci. July 1980, which is incorporated by reference in its entirety.

In some embodiments, more than one pharmaceutically active component may be included in the composition. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof. Suitable actives for use in the compositions herein include, but are not limited to, the following therapeutic classes: ace-inhibitor; adrenergic agent; adrenocortical steroid; adrenocortical suppressant; aldosterone antagonist; alkaloid; amino acid; anabolic; analeptic; analgesic; anesthetic; anorectic; anti-acne agent; anti-adrenergic; anti-allergic; anti-amebic; anti-anemic; anti-anginal; anti-anxiety; anti-arthritic; anti-arrythmia; anti-asthmatic; anti-atherosclerotic; anti-cholesterolemic; antibacterial; antibiotic; anticholinergic; anticoagulant; anticonvulsant; antidepressant; antidiabetic; antidiarrheal; antidiuretic; antidote; anti-emetic; anti-epileptic; antifibrinolytic; antifungal; antihemorrhagic; antihistamine; antihyperlipidemia; antihypertensive; antihypotensive; anti-infective (both systemic and non-systemic); anti-inflammatory; anti-lipid; anti-manic; antimicrobial; antimigraine; antimitotic; antimycotic, antinauseant; antineoplastic; antineutropenic; anti-obesity; antiparasitic; anti-parkinson; antiproliferative; antipsychotic; anti-pyretic; antirheumatic; antiseborrheic; antisecretory; antispasmodic; anti-stroke; antithrombotic; anti-thyroid; anti-tumor; anti-tussive; anti-ulcerative; anti-uricemic; antiviral; appetite suppressant; appetite stimulant; biological response modifier; blood glucose regulator; blood modifier; blood metabolism regulator; bone resorption inhibitor; bronchodilator; cardiovascular agent; central nervous system stimulant; cerebral dilator; contraceptive; coronary dilator; cholinergic; cough suppressant; decongestant; depressant; diagnostic aid; dietary supplement; diuretic; dopaminergic agent; enzymes; estrogen receptor agonist; endometriosis management agent; expectorant; erectile dysfunction therapy; erythropoietic; ibrinolytic; fertility agent; fluorescent agent; free oxygen radical scavenger; gastric acid suppressant; gastrointestinal motility effector; genetic modifier; glucocorticoid; hair growth stimulant; hemostatic; histamine H2 receptor antagonists; homeopathic remedy; hormone; hypercalcemia management agent; hypocalcemia management agent; hypocholesterolemic; hypoglycemic; hypolipidemic; hypotensive; ion exchange resin; imaging agent; immunizing agent; immunomodulator; immunoregulator; immunostimulant; immunosuppressant; keratolytic; laxative; LHRH agonist; mood regulator; motion sickness preparation; mucolytic; muscle relaxant; mydriatic; nasal decongestant; neuromuscular blocking agent; neuroprotective; NMDA antagonist; non-hormonal sterol derivative; osteoporosis therapy; oxytocic; parasympatholytic; parasympathomimetic; plasminogen activator; platelet activating factor antagonist; platelet aggregation inhibitor; prostaglandin; psychotherapeutic; psychotropic; radioactive agent; respiratory agent; scabicide; sclerosing agent; sedative; sedative-hypnotic; selective adenosine A1 antagonist; serotonin antagonist; serotonin inhibitor; serotonin receptor antagonist; smoking cessation therapy; steroid; stimulant; sympatholytic; terine relaxant; thyroid hormone; thyroid inhibitor; thyromimetic; tranquilizer; tremor therapy; amyotrophic lateral sclerosis agent; cerebral ischemia agent; Paget's disease agent; unstable angina agent; vasoconstrictor; vasodilator; weight management; wound healing agent; xanthine oxidase inhibitor; and combinations thereof.

Examples of actives suitable for use herein include antacids, H2-antagonists, and analgesics. For example, antacid dosages can be prepared using the ingredients calcium carbonate alone or in combination with magnesium hydroxide, and/or aluminum hydroxide. Moreover, antacids can be used in combination with H2-antagonists.

Analgesics include opiates and opiate derivatives, such as oxycodone (commercially available as Oxycontin®); ibuprofen (commercially available as Motrin®, Advil®, Motrin Children's®, Motrin IB®, Advil Children's®, Motrin Infants'®, Motrin Junior®, Ibu-2®, Proprinal®, Ibu-200®, Midol Cramp Formula®, Bufen®, Motrin Migraine Pain®, Addaprin® and Haltran®), aspirin (commercially available as Empirin®, Ecotrin®, Genuine Bayer®, and Halfprin®), acetaminophen (commercially available as Silapap Infant's®, Silapap Children's®, Tylenol®, Tylenol Children's®, Tylenol Extra Strength®, Tylenol Infants' Original®, Tylenol Infants'®, Tylenol Arthritis®, T-Painol®, Q-Pap®, Cetafen®, Dolono®, Tycolene®, APAP® and Aminofen®), and combinations thereof that may optionally include caffeine. Other pain relieving agents may be used in the present invention, including meperidine hydrochloride (commercially available as Demerol®), capsaicin (commercially available as Qutenza®), morphine sulfate and naltrexone hydrochloride (commercially available as Embeda®), hydromorphone hydrochloride (commercially available as Dilaudid®), propoxyphene napsylate and acetaminophen (commercially available as Darvocet-N®), Fentanyl (commercially available as Duragesic®, Onsolis®, and Fentora®), sodium hyaluronate (commercially available as Euflexxa®), adalimumab (commercially available as Humira®), sumatriptan succinate (commercially available as Imitrex®), fentanyl iontophoretic (commercially available as Ionsys®), orphenadrine citrate (commercially available as Norgesic®), magnesium salicylate tetrahydrate (commercially available as Novasal®), oxymorphone hydrochloride (commercially available as Opana ER®), methocarbamol (commercially available as Robaxin®), carisoprodol (commercially available as Soma®), tramadol hydrochloride (commercially available as Ultracet® and Ultram®), morphine sulfate (commercially available as MS Contin®), metaxalone (commercially available as Skelaxin®), oxycodone hydrochloride (commercially available as OxyContin®), acetaminophen/oxycodone hydrochloride (commercially available as Percocet®), oxycodone/aspirin (commercially available as Percodan®), hydrocodone bitartrate/acetaminophen (commercially available as Vicodin®), hydrocodone bitartrate/ibuprofen (commercially available as Vicoprofen®), nepafenac (commercially available as Nevanac®), and pregabalin (commercially available as Lyrica®).

The compositions disclosed herein may further include agents such as NSAIDs, including etodolac (commercially available as Lodine®), ketorolac tromethamine (commercially available as Acular® or Acuvail®), naproxen sodium (commercially available as Anaprox®, Naprosyn®), flurbiprofen (commercially available as Ansaid®), diclofenac sodium/misoprostol (commercially available as Arthrotec®), celecoxib (commercially available as Celebrex®), sulindac (commercially available as Clinoril®), oxaprozin (commercially available as Daypro®), piroxicam (commercially available as Feldene®), indomethacin (commercially available as Indocin®), meloxicam (commercially available as Mobic®), mefenamic acid (commercially available as Ponstel®), tolmetin sodium (commercially available as Tolectin®), choline magnesium trisalicylate (commercially available as Trilisate®), diclofenac sodium (commercially available as Voltaren®), diclofenac potassium (commercially available as Cambia® or Zipsor®), and misoprostol (commercially available as Cytotec®). Opiate agonists and antagonists, such as buprenorphine and naloxone are further examples of drugs for use in the present invention.

Other drugs for other actives for use herein include anti-diarrheals such as loperamide (commercially available as Imodium AD®, Imotil®, Kaodene®, Imperim®, Diamode®, QC Anti-Diarrheal®, Health Care America Anti-Diarrheal®, Leader A-D®, and Imogen®), nitazoxanide (commercially available as Alinia®) and diphenoxylate hydrochloride/atropine sulfate (commercially available as Lomotil®), anti-histamines, anti-tussives, decongestants, vitamins, and breath fresheners. Common drugs used alone or in combination for colds, pain, fever, cough, congestion, runny nose and allergies, such as acetaminophen, ibuprofen, chlorpheniramine maleate, dextromethorphan, dextromethorphan HBr, phenylephrine HCl, pseudoephedrine HCl, diphenhydramine and combinations thereof, such as dextromethophan HBr and phenylephrine HCl (available as Triaminic®) may be included in the compositions of the present invention.

Other actives useful herein include, but are not limited to, alcohol dependence treatment, such as acamprosate calcium (commercially available as Campral®); Allergy treatment medications, such as promethazine hydrochloride (commercially available as Phenergan®), bepotastine besilate (commercially available as Bepreve®), hydrocodone polistirex/chlorpheniramine polistirex (commercially available as Tussionex®), cetirizine hydrochloride (commercially available as Zyrtec®), cetirizine hydrochloride/pseudoephedrine hydrochloride (commercially available as Zyrtec-D®), promethazine hydrochloride/codeine phosphate (commercially available as Phenergan® with Codeine), pemirolast (commercially available as Alamast®), fexofenadine hydrochloride (commercially available as Allegra®), meclizine hydrochloride (commercially available as Antivert®), azelastine hydrochloride (commercially available as Astelin®), nizatidine (commercially available as Axid®), desloratadine (commercially available as Clarinex®), cromolyn sodium (commercially available as Crolom®), epinastine hydrochloride (commercially available as Elestat®), azelastine hydrochloride (commercially available as Optivar®), prednisolone sodium phosphate (commercially available as Orapred ODT®), olopatadine hydrochloride (commercially available as Patanol®), ketotifen fumarate (commercially available as Zaditor®), and montelukast sodium (commercially available as Singulair®); and anti-histamines such as diphenhydramine HCl (available as Benadryl®), loratadine (available as Claritin®), astemizole (available as Hismanal®), nabumetone (available as Relafen®), diphenydramine HCL (available as TheraFlu®) and clemastine (available as Tavist®).

Compositions of the present disclosure may further include Alzheimer's treatment medications, such as tacrine hydrochloride (commercially available as Cognex®), galantamine (commercially available as Razadyne®), donepezil hydrochloride (commercially available as Aricept®), rivastigmine tartrate (commercially available as Exelon®), caprylidene (commercially available as Axona®), and memantine (commercially available as Namenda®); anemia medication, such as cyanocobalamin (commercially available as Nascobal®) and ferumoxytol (commercially available as Feraheme®); anesthetics, such as antipyrine with benzocaine (commercially available as Auralgan®, Aurodex® and Auroto®); angina medication, such as amlodipine besylate (commercially available as Norvasc®), nitroglycerin (commercially available as Nitro-Bid®, Nitro-Dur®, Nitrolingual®, Nitrostat®, Transderm-Nitro®), isosorbide mononitrate (commercially available as Imdur®), and isosorbide dinitrate (commercially available as Isordil®); anti-tussives such as guaifensin; anti-Alzheimer's agents, such as nicergoline; and Ca^{H}-antagonists such as nifedipine (commercially available as Procardia® and Adalat®).

Actives useful in the present disclosure may also include anti-asthmatics, such as albuterol sulfate (commercially available as Proventil®), ipratropium bromide (commercially available as Atrovent®), salmeterol xinafoate (commercially available as Serevent®), zafirlukast (commercially available as Accolate®), flunisolide (commercially available as AeroBid®), metaproterenol sulfate (commercially available as Alupent®), albuterol inhalation (commercially available as Ventolin®), terbutaline sulfate (commercially available as Brethine®), formoterol (commercially available as Foradil®), cromolyn sodium (commercially available as Intal®), levalbuterol hydrochloride (commercially available as Xopenex®), zileuton (commercially available as Zyflo®), fluticasone propionate/salmeterol (commercially available as Advair®), albuterol sulfate/triamcinolone acetonide (commercially available as Azmacort®), dimethylxanthine (commercially available as Theophylline®), and beclomethasone (commercially available as Beclovent®, Beconase®, Qvar®, Vancenase®, Vanceril®); angioedema medication, such as Cl esterase Inhibitor (human) (commercially available as Berinert®) and ecallantide (commercially available as Kalbitor®); and antibacterial medications, such as trimethoprim/sulfamethoxazole (commercially available as Bactrim®), mupirocin (commercially available as Bactroban®), metronidazole (commercially available as Flagyl®), sulfisoxazole acetyl (commercially available as Gantrisin®), bismuth subsalicylate and metronidazole/tetracycline hydrochloride (commercially available as Helidac Therapy®), nitrofurantoin (commercially available as Macrodantin®), norfloxacin (commercially available as Noroxin®), erythromycin ethylsuccinate/Sulfisoxazole acetyl (commercially available as Pediazole®), and levofloxacin (commercially available as Levaquin®).

The compositions of the present disclosure may further include one or more antibiotics, including amoxicillin (commercially available as Amoxil®), ampicillin (commercially available as Omnipen®, Polycillin® and Principen®), amoxicillin/clavulanate potassium (commercially available as Augmentin®), moxifloxacin hydrochloride (commercially available as Avelox®), besifloxacin (commercially available as Besivance®), clarithromycin (commercially available as Biaxin®), ceftibuten (commercially available as Cedax®), cefuroxime axetil (commercially available as Ceftin®), cefprozil (commercially available as Cefzil®), ciprofloxacin hydrochloride (commercially available as Ciloxan® and Cipro®), clindamycin phosphate (commercially available as Cleocin T®), doxycycline hyclate (commercially available as Doryx®), dirithromycin (commercially available as Dynabac®), erythromycin (commercially available as E.E.S. ®, E-Mycin®, Eryc®, Ery-Tab®, Erythrocin®, and PCE®), erythromycin topical (commercially available as A/T/S®, Erycette®, T-Stat®), gemifloxacin (commercially available as Factive®), ofloxacin (commercially known as Ocuflox®, Floxin®), telithromycin (commercially available as Ketek®), lomefloxacin hydrochloride (commercially available as Maxaquin®), minocycline hydrochloride (commercially available as Minocin®), fosfomycin tromethamine (commercially available as Monurol®), penicillin with potassium (commercially available as Penicillin VK®, Veetids®), trimethoprim (commercially available as Primsol®), ciprofloxacin hydrochloride (commercially available as Proquin XR®), rifampin, isoniazid and pyrazinamide (commercially available as Rifater®), cefditoren (commercially available as Spectracef®), cefixime (commercially available as Suprax®), tetracycline (commercially available as Achromycin V® and Sumycin®), tobramycin (commercially available as Tobrex®), rifaximin (commercially available as Xifaxan®), azithromycin (commercially available as Zithromax®), azithromycin suspension (commercially available as Zmax®), linezolid (commercially available as Zyvox®), benzoyl peroxide and clindamycin (commercially available as BenzaClin®), erythromycin and benzoyl peroxide (commercially available as Benzamycin®), dexamethasone (commercially available as Ozurdex®), ciprofloxacin and dexamethasone (commercially available as Ciprodex®), polymyxin B sulfate/neomycin sulfate/hydrocortisone (commercially available as Cortisporin®), colistin sulfate/neomycin sulfate/hydrocortisone acetate/thonzonium bromide (commercially available as Cortisporin-TC Otic®), cephalexin hydrochloride (commercially available as Keflex®), cefdinir (commercially available as Omnicef®), and gatifloxacin (commercially available as Zymar®).

The compositions included herein may also be useful for the treatment of urticarial. Urticaria is a common skin disease characterized by pruritic wheal and flare-type skin reactions—with or without angioedema—that usually persists less than 24 hours. Chronic urticaria (CU) is defined by recurrent episodes occurring at least twice a week for 6 weeks or more. Chronic urticaria can be classified into two caterogies: (1) Chronic Inducible (CIndU) also known as Physical Urticaria that results from a specific environmental stimuli or trigger; or (2) Chronic Spontaneous Urticaria (CSU) also known as Chronic Idiopathic Urticaria where the trigger is unknown. CU prevalence in the US is currently estimated to be 0.5% to 1% of the total population. Although all age groups can be affected, the peak incidence of urticaria occurs between 20 and 40 years of age. The duration of the disease is generally 1-5 years, but is likely to last longer in more severe cases. In children (ages <18 years), the prevalence varies from less than 1% to almost 5%.

Other useful actives include cancer treatment medications, including cyclophosphamide (commercially available as Cytoxan®), methotrexate (commercially available as Rheumatrex® and Trexal®), tamoxifen citrate (commercially available as Nolvadex®), bevacizumab (commercially available as Avastin®), everolimus (commercially available as Afinitor®), pazopanib (commercially available as Votrient®), and anastrozole (commercially available as Arimidex®); leukemia treatment, such as ofatumumab (commercially available as Arzerra®); anti-thrombotic drugs, such as antithrombin recombinant lyophilized powder (commercially available as Atryn®), prasugrel (commercially available as Efient®); anti-coagulants, such as aspirin with extended-release dipyridamole (commercially available as Aggrenox®), warfarin sodium (commercially available as Coumadin®), dipyridamole (commercially available as Persantine®), dalteparin (commercially available as Fragmin®), danaparoid (commercially available as Orgaran®), enoxaparin (commercially available as Lovenox®), heparin (commercially available as Hep-Lock, Hep-Pak, Hep-Pak CVC, Heparin Lock Flush), tinzaparin (commercially available as Innohep®), and clopidogrel bisulfate (commercially available as Plavix®); antiemetics, such as granisetron hydrochloride (commercially available as Kytril®) and nabilone (commercially available as Cesamet®), trimethobenzamide hydrochloride (commercially available as Tigan®), and ondansetron hydrochloride (commercially available as Zofran®); anti-fungal treatment, such as ketoconazole (commercially available as Nizoral®), posaconazole (commercially available as Noxafil®), ciclopirox (commercially available as Penlac®), griseofulvin (commercially available as Gris-PEG®), oxiconazole nitrate (commercially available as Oxistat®), fluconazole (commercially available as Diflucan®), sertaconazole nitrate (commercially available as Ertaczo®), terbinafine hydrochloride (commercially available as Lamisil®), ciclopirox (commercially available as Loprox®), nystatin/triamcinolone acetonide (commercially available as Mycolog-II®), econazole nitrate (commercially available as Spectazole®), itraconazole (commercially available as Sporanox®), and terconazole (commercially available as Terazol®).

Actives may further include anti-inflammatory medications, such as hydroxychloroquine sulfate (commercially available as Plaquenil®), fluticasone propionate (commercially available as Cutivate®), canakinumab (commercially available as Llaris®), amcinonide (commercially available as Cyclocort®), methylprednisolone (commercially available as Medrol®), budesonide (commercially available as Entocort EC®), anakinra (commercially available as Kineret®), diflorasone diacetate (commercially available as Psorcon®), and etanercept (commercially available as Enbrel®); antispasmodic medication, such as phenobarbital/hyoscyamine sulfate/atropine sulfate/scopolamine hydrobromide (commercially available as Donnatal®); antiviral treatment, such as oseltamivir phosphate (commercially available as Tamiflu®); anti-parasites medication, including tinidazole (commercially available as Tindamax®); appetite treatment mediations, such as megestrol acetate (commercially available as Megace ES®), phentermine hydrochloride (commercially available as Adipex-P®), and diethylpropion hydrochloride (commercially available as Tenuate®); arthritis medications, including leflunomide (commercially available as Arava®), certolizumab pegol (commercially available as Cimzia®), diclofenac sodium (commercially available as Pennsaid®), golimumab (commercially available as Simponi®), and tocilizumab (commercially available as Actemra®); bladder control medication, such as trospium chloride (commercially available as Sanctura®), desmopressin acetate (commercially available as DDAVP®), tolterodine tartrate (commercially available as Detrol®), oxybutynin chloride (commercially available as Ditropan® or Gelnique®), darifenacin (commercially available as Enablex®), and solifenacin succinate (commercially available as VESIcare®); blood vessel constrictors, such as methylergonovine maleate (commercially available as Methergine®); plasma uric managers, such as rasburicase (commercially available as Elitek®); iron deficiency anemia medications, such as ferumoxytol (commercially available as Feraheme®); lymphoma medications, such as pralatrexate (commercially available as Folotyn®), romidepsin (commercially available as Isodax®); malaria medication, such as artemether/lumefantrine (commercially available as Coartem®); hyponatremia medication, such as tolvatpan (commercially available as Samsca®); medication for treatment of von Willebrand disease (commercially available as Wilate®); anti-hypertension medications, such as treprostinil (commercially available as Tyvaso®), tadalafil (commercially available as Adcirca®); cholesterol lowering medication, including paricalcitol (commercially available as Altocor®), pitavastatin (commercially available as Livalo®), lovastatin, niacin (commercially available as Advicor®), colestipol hydrochloride (commercially available as Colestid®), rosuvastatin calcium (commercially available as Crestor®), fluvastatin sodium (commercially available as Lescol®), atorvastatin calcium (commercially available as Lipitor®), lovastatin (commercially available as Mevacor®), niacin (commercially available as Niaspan®), pravastatin sodium (commercially available as Pravachol®), pavastatin sodium with buffered aspirin (commercially available as Pravigard PAC®), cholestyramine (commercially available as Questran®), simvastatin and niacin (commercially available as Simcor®), atenolol, chlorthalidone (commercially available as Tenoretic®), atenolol (commercially available as Tenormin®), fenofibrate (commercially available as Tricor®), fenofibrate (commercially available as Triglide®), ezetimibe/simvastatin (commercially available as Vytorin®), colesevelam (commercially available as WelChol®), bisoprolol fumarate (commercially available as Zebeta®), ezetimibe (commercially available as Zetia®), bisoprolol fumarate/hydrochlorothiazide (commercially available as Ziac®), and simvastatin (commercially available as Zocor®).

The actives included herein may also include chronic kidney disease medication, such as paricalcitol (commercially available as Zemplar®); contraceptive agents, including etonogestrel (commercially available as Implanon®), norethindrone acetate, ethinyl estradiol (commercially available as Loestrin 24 FE®), ethinyl estradiol, norelgestromin (commercially available as Ortho Evra®), levonorgestrel (commercially available as Plan B®), levonorgestrel and ethinyl estradiol (commercially available as Preven®), levonorgestrel, ethinyl estradiol (commercially available as Seasonique®), and medroxyprogesterone acetate (commercially available as Depo-Provera®); COPD medication, such as arformoterol tartrate (commercially available as Brovana®) and ipratropium bromide, albuterol sulfate (commercially available as Combivent®); cough suppressants, including benzonatate (commercially available as Tessalon®), guaifenesin, codeine phosphate (commercially available as Tussi-Organidin NR®), and acetaminophen, codeine phosphate (commercially available as Tylenol with Codeine®); medication for the treatment of diabetes, including pioglitazone hydrochloride, metformin hydrochloride (commercially available as ACTOplus met®), bromocriptine mesylate (commercially available as Cycloset®), liraglutide (commercially available as Victoza®), saxagliptin (commercially available as Onglyza®), pioglitazone hydrochloride (commercially available as Actos®), glimepiride (commercially available as Amaryl®), rosiglitazone maleate, metformin hydrochloride (commercially available as Avandamet®), rosiglitazone maleate (commercially available as Avandaryl®), rosiglitazone maleate (commercially available as Avandia®), exenatide (commercially available as Byetta®), exenatide (commercially available as Bydureon®), chlorpropamide (commercially available as Diabinese®), pioglitazone hydrochloride, glimepiride (commercially available as Duetact®), metformin hydrochloride (commercially available as Glucophage®), glipizide (commercially available as Glucotrol®), glyburide, metformin (commercially available as Glucovance® and Fortamet®), metformin hydrochloride (commercially available as Glumetza®), sitagliptin (commercially available as Januvia®), detemir (commercially available as Levemir®), glipizide, metformin hydrochloride (commercially available as Metaglip®), glyburide (commercially available as Micronase®), repaglinide (commercially available as Prandin®), acarbose (commercially available as Precose®), nateglinide (commercially available as Starlix®), pramlintide acetate (commercially available as Symlin®), canagliflozin (commercially available as Invokana®), linagliptin (commercially available as Tradjenta®), dapagliflozin (commercially available as Farxiga®), insulin glargine (commercially available as Lantus® or Toujeo®), insulin aspart (commercially available as Novolog®), insulin lispro, empagliflozin (commercially available as Jardiance®), and tolazamide (commercially available as Tolinase®).

Other useful actives may include digestive agents, such as sulfasalazine (commercially available as Azulfidine®), rabeprazole sodium (commercially available as AcipHex®), lubiprostone (commercially available as Amitiza®), dicyclomine hydrochloride (commercially available as Bentyl®), sucralfate (commercially available as Carafate®), lactulose (commercially available as Chronulac®), docusate (commercially available as Colace®), balsalazide disodium (commercially available as Colazal®), losartan potassium (commercially available as Cozaar®), olsalazine sodium (commercially available as Dipentum®), chlordiazepoxide hydrochloride, clidinium bromide (commercially available as Librax®), esomeprazole magnesium (commercially available as Nexium®), famotidine (commercially available as Pepcid®), lansoprazole (commercially available as Prevacid®), lansoprazole and naproxen (commercially available as Prevacid NapraPAC®), amoxicillin/clarithromycin/lansoprazole (commercially available as Prevpac®), omeprazole (commercially available as Prilosec®), pantoprazole sodium (commercially available as Protonix®), metoclopramide hydrochloride (commercially available as Reglan® or Metozolv®), cimetidine (commercially available as Tagamet®), ranitidine hydrochloride (commercially available as Zantac®), and omeprazole, sodium bicarbonate (commercially available as Zegerid®); diuretics, including spironolactone, hydrochlorothiazide (commercially available as Aldactazide®), spironolactone (commercially available as Aldactone®), bumetanide (commercially available as Bumex®), torsemide (commercially available as Demadex®), chlorothiazide (commercially available as Diuril®), furosemide (commercially available as Lasix®), metolazone (commercially available as Zaroxolyn®), and hydrochlorothiazide, triamterene (commercially available as Dyazide®).

Actives useful herein may also include treatment for emphysema, such as tiotropium bromide (commercially available as Spiriva®); fibromyalgia medication, such as milnacipran hydrochloride (commercially available as Savella®); medication for the treatment of gout, such as colchicine (commercially available as Colcrys®), and febuxostat (commercially available as Uloric®); enema treatments, including aminosalicylic acid (commercially available as Mesalamine® and Rowasa®); epilepsy medications, including valproic acid (commercially available as Depakene®), felbamate (commercially available as Felbatol®), lamotrigine (commercially available as Lamictal®), primidone (commercially available as Mysoline®), oxcarbazepine (commercially available as Trileptal®), zonisamide(commercially available as Zonegran®), levetiracetam (commercially available as Keppra®), and phenytoin sodium (commercially available as Dilantin®).

Actives useful herein may further include eye medications and treatment, such as dipivefrin hydrochloride (commercially available as Propine®), valganciclovir (commercially available as Valcyte®), ganciclovir ophthalmic gel (commercially available as Zirgan®); bepotastine besilate (commercially available as Bepreve®), besifloxacin (commercially available as Besivance®), bromfenac (commercially available as Xibrom®), fluorometholone (commercially available as FML®), pilocarpine hydrochloride (commercially available as Pilocar®), cyclosporine (commercially available as Restasis®), brimonidine tartrate (commercially available as Alphagan P®), dorzolamide hydrochloride/timolol maleate (commercially available as Cosopt®), bimatoprost (commercially available as Lumigan®), timolol maleate (available as Timoptic®), travoprost (commercially available as Travatan®), latanoprost (commercially available as Xalatan®), echothiophate iodide (commercially available as Phospholine Iodide®), and ranibizumab (commercially available as Lucentis®); fluid controllers, such as acetazolamide (commercially available as Diamox®); gallstone medications, including ursodiol (commercially available as Actigall®); medication for the treatment of gingivitis, including chlorhexidine gluconate (commercially available as Peridex®); headache medications, including butalbital/codeine phosphate/aspirin/caffeine (commercially available as Fiornal® with Codeine), naratriptan hydrochloride (commercially available as Amerge®), almotriptan (commercially available as Axert®), ergotamine tartrate/caffeine (commercially available as Cafergot®), butalbital/acetaminophen/caffeine (commercially available as Fioricet®), butalbital/aspirin/caffeine (commercially available as Fiorinal®), frovatriptan succinate (commercially available as Frova®), rizatriptan benzoate (commercially available as Maxalt®), isometheptene mucate/dichloralphenazone/acetaminophen (commercially available as Midrin®), dihydroergotamine mesylate (commercially available as Migranal®), eletriptan hydrobromide (commercially available as Relpax®), and zolmitriptan (commercially available as Zomig®); influenza medication, such as haemophilus b conjugate vaccine; tetanus toxoid conjugate (commercially available as Hiberix®); and heart treatments, including quinidine sulfate, isosorbide dinitrate/hydralazine hydrochloride (commercially available as BiDil®), digoxin (commercially available as Lanoxin®), flecainide acetate (commercially available as Tambocor®), mexiletine hydrochloride (commercially available as Mexitil®), disopyramide phosphate (commercially available as Norpace®), procainamide hydrochloride (commercially available as Procanbid®), and propafenone (commercially available as Rythmol®).

Other useful actives include hepatitis treatments, including entecavir (commercially available as Baraclude®), hepatitis B immune globulin (commercially available as HepaGam B®), and copegus/rebetol/ribasphere/vilona/virazole (commercially available as Ribavirin®); herpes treatments, including valacyclovir hydrochloride (commercially available as Valtrex®), penciclovir (commercially available as Denavir®), acyclovir (commercially available as Zovirax®), and famciclovir (commercially available as Famvir®); treatment for high blood pressure, including enalaprilat (available as Vasotec®), captopril (available as Capoten®) and lisinopril (available as Zestril®), verapamil hydrochloride (available as Calan®), ramipril (commercially available as Altace®), olmesartan medoxomil (commercially available as Benicar®), amlodipine/atorvastatin (commercially available as Caduet®), nicardipine hydrochloride (commercially available as Cardene®), diltiazem hydrochloride (commercially available as Cardizem®), quinapril hydrochloride (commercially available as Accupril®), quinapril hydrochloride/hydrochlorothiazide (commercially available as Accuretic®), perindopril erbumine (commercially available as Aceon®), candesartan cilexetil (commercially available as Atacand®), candesartan cilexetil/hydrochlorothiazide (commercially available as Atacand HCT®), irbesartan/hydrochlorothiazide (commercially available as Avalide®), irbesartan (commercially available as Avapro®), amlodipine besylate/olmesartan medoxomil (commercially available as Azor®), levobunolol hydrochloride (commercially available as Betagan®), betaxolol hydrochloride (commercially available as Betoptic®), nebivolol (commercially available as Bystolic®), captopril/hydrochlorothiazide (commercially available as Capozide®), doxazosin mesylate (commercially available as Cardura®), clonidine hydrochloride (commercially available as Catapres®), carvedilol (commercially available as Coreg®), nadolol (commercially available as Corgard®), nadolol/bendroflumethiazide (commercially available as Corzide®), valsartan (commercially available as Diovan®), isradipine (commercially available as DynaCirc®), Guanabenz acetate. (commercially available as Wytensin ®), Guanfacine hydrochloride (commercially available as Tenex ® or Intuniv®), losartan potassium/hydrochlorothiazide (commercially available as Hyzaar®), propranolol hydrochloride (commercially available as Indera®), propranolol hydrochloride/hydrochlorothiazide (commercially available as Inderide®), eplerenone (commercially available as Inspra®), ambrisentan (commercially available as Letairis®), enalapril maleate/felodipine (commercially available as Lexxel®), metoprolol tartrate (commercially available as Lopressor®), benazepril hydrochloride (commercially available as Lotensin®), benazepril hydrochloride/hydrochlorothiazide (commercially available as Lotensin HCT®), amlodipine/benazepril hydrochloride (commercially available as Lotrel®), indapamide (commercially available as Lozol®), trandolapril (commercially available as Mavik®), telmisartan (commercially available as Micardis®), telmisartan/hydrochlorothiazide (commercially available as Micardis HCT®), prazosin hydrochloride (commercially available as Minipress®), amiloride, hydrochlorothiazide (commercially available as Moduretic®), fosinopril sodium (commercially available as ZZXT Monopril®), fosinopril sodium/hydrochlorothiazide (commercially available as Monopril-HCT®), pindolol (commercially available as Visken®), felodipine (commercially available as Plendil®), sildenafil citrate (commercially available as Revatio®), Nisoldipine (commercially available as Sular®), trandolapril/verapamil hydrochloride (commercially available as Tarka®), aliskiren (commercially available as Tekturna®), eprosartan mesylate (commercially available as Teveten®), eprosartan mesylate/hydrochlorothiazide (commercially available as Teveten HCT®), moexipril hydrochloride/hydrochlorothiazide (commercially available as Uniretic®), moexipril hydrochloride (commercially available as Univasc®), enalapril maleate/hydrochlorothiazide (commercially available as Vaseretic®), and lisinopril/hydrochlorothiazide (commercially available as Zestoretic®).

The compositions of the present disclosure may include actives useful in the medication for the treatment of HIV/AIDS, such as amprenavir (commercially available as Agenerase®), tipranavir (commercially available as Aptivus®), efavirenz/emtricitabine/tenofovir (commercially available as Atripla®), lamivudine/zidovudine (commercially available as Combivir®), indinavir sulfate (commercially available as Crixivan®), lamivudine (commercially available as Epivir®), saquinavir (commercially available as Fortovase®), zalcitabine (commercially available as Hivid®), lopinavir/ritonavir (commercially available as Kaletra®), fosamprenavir calcium (commercially available as Lexiva®), ritonavir (commercially available as Norvir®), zidovudine (commercially available as Retrovir®), atazanavir sulfate (commercially available as Reyataz®), efavirenz (commercially available as Sustiva®), abacavir/lamivudine/zidovudine (commercially available as Trizivir®), didanosine (commercially available as Videx®), nelfinavir mesylate (commercially available as Viracept®), nevirapine (commercially available as Viramune®), tenofovir disoproxil fumarate (commercially available as Viread®), stavudine (commercially available as Zerit®), and abacavir sulfate (commercially available as Ziagen®); homocysteiene removers, including betaine anhydrous (commercially available as Cystadane®); medications, such as insulin (commercially available as Apidra®, Humalog®, Humulin®, Iletin®, Tresiba®, and Novolin®); and HPV treatment, such as Human papillomavirus vaccine (commercially available as Gardasil®) or human papillomavirus bivalent (commercially available as Cervarix®); immunosuppressants, including cyclosporine (commercially available as Gengraf®, Neoral®, Sandimmune®, and Apo-Cyclosporine®).

Actives useful in the present disclosure may further include prolactin inhibitors, such as bromocriptine mesylate (commercially available as Parlodel®); medications for aiding in stress tests, such as regadenoson (commercially available as Lexiscan®); baldness medication, including finasteride (commercially available as Propecia® and Proscar®); pancreatitis treatment, such as gemfibrozil (commercially available as Lopid®); hormone medications, such as norethindrone acetate/ethinyl estradiol (commercially available as femHRT®), goserelin acetate (commercially available as Zoladex®), progesterone gel (commercially available as Prochieve®), progesterone (commercially available as Prometrium®), calcitonin-salmon (commercially available as Miacalcin®), calcitriol (commercially available as Rocaltrol®), synthroid (commercially available as Levothroid®, Levoxyl®, Unithroid®), testosterone (commercially available as Testopel®, Androderm®, Testoderm®, and AndroGel®); menopause medication, such as estradiol/norethindrone acetate (commercially available as Activella®), drospirenone/estradiol (commercially available as Angeliq®), estradiol/levonorgestrel (commercially available as Climara Pro®), estradiol/norethindrone acetate (commercially available as CombiPatch®), estradiol (commercially available as Estrasorb®, Vagifem® and EstroGel®), esterified estrogens and methyltestosterone (commercially available as Estratest®), estrogen (commercially available as Alora®, Climara®, Esclim®, Estraderm®, Vivelle®, Vivelle-Dot®), estropipate (commercially available as Ogen®), conjugated estrogens (commercially available as Premarin®), and medroxyprogesterone acetate (commercially available as Provera®); menstrual medications, including leuprolide acetate (commercially available as Lupron Depot), tranexamic acid (commercially available as Lysteda®), and norethindrone acetate (commercially available as Aygestin®); and muscle relaxants, including cyclobenzaprine hydrochloride (commercially available as Flexeril®), tizanidine (commercially available as Zanaflex®), and hyoscyamine sulfate (commercially available as Levsin®).

Actives useful herein may also include osteoporosis medications, including ibrandronate sodium (commercially available as Boniva®), risedronate (commercially available as Actonel®), raloxifene hydrochloride (commercially available as Evista®, Fortical®), and alendronate sodium (commercially available as Fosamax®); ovulation enhancers, including clomiphene citrate (commercially available as Serophene®, Clomid®, Serophene®); Paget's disease treatment, such as etidronate disodium (commercially available as Didronel®); pancreatic enzyme deficiency medications, such as pancrelipase (commercially available as Pancrease® or Zenpep®); medication for the treatment of Parkinson's disease, such as pramipexole dihydrochloride (commercially available as Mirapex®), ropinirole hydrochloride (commercially available as Requip®), carbidopa/levodopa (commercially available as Sinemet CR®), carbidopa/levodopa/entacapone (commercially available as Stalevo®), selegiline hydrochloride (commercially available as Zelapar®), rasagiline (commercially available as Azilect®), entacapone (commercially available as Comtan®), and selegiline hydrochloride (commercially available as Eldepryl®); multiple sclerosis medication, such as dalfampridine (commercially available as Ampyra®) and interferon beta-I b (commercially available as Extavia®); prostate medication, including flutamide (commercially available as Eulexin®), nilutamide (commercially available as Nilandron®), dutasteride (commercially available as Avodart®), tamsulosin hydrochloride (commercially available as Flomax®), terazosin hydrochloride (commercially available as Hytrin®), and alfuzosin hydrochloride (commercially available as UroXatral®).

Compositions of the present disclosure may further include psychiatric medications, including alprazolam (available as Niravam®, Xanax®), clozopin (available as Clozaril®), haloperidol (available as Haldol®), fluoxetine hydrochloride (available as Prozac®), sertraline hydrochloride (available as Zoloft®), asenapine (commercially available as Saphris®), iloperidone (commercially available as Fanapt®), paroxtine hydrochloride (available as Paxil®), aripiprazole (commercially available as Abilify®), guanfacine (commercially available as Intuniv®), Amphetamines and methamphetamines (commercially available as Adderall® and Desoxyn®), clomipramine hydrochloride (commercially available as Anafranil®), Buspirone hydrochloride (commercially available as BuSpar®), citalopram hydrobromide (commercially available as Celexa®), duloxetine hydrochloride (commercially available as Cymbalta®), methylphenidate (commercially available as Ritalin, Daytrana®), divalproex sodium (Valproic acid) (commercially available as Depakote®), dextroamphetamine sulfate (commercially available as Dexedrine®), venlafaxine hydrochloride (commercially available as Effexor®), selegiline (commercially available as Emsam®), carbamazepine (commercially available as Equetro®), lithium carbonate (commercially available as Eskalith®), fluvoxamine maleate/dexmethylphenidate hydrochloride (commercially available as Focalin®), ziprasidone hydrochloride (commercially available as Geodon®), ergoloid mesylates (commercially available as Hydergine®), escitalopram oxalate (commercially available as Lexapro®), chlordiazepoxide (commercially available as Librium®), molindone hydrochloride (commercially available as Moban®), phenelzine sulfate (commercially available as Nardil®), thiothixene (commercially available as Navane®), desipramine hydrochloride (commercially available as Norpramin®), benzodiazepines (such as those available as Oxazepam®), nortriptyline hydrochloride (commercially available as Pamelor®), tranylcypromine sulfate (commercially available as Parnate®), prochlorperazine, mirtazapine (commercially available as Remeron®), risperidone (commercially available as Risperdal®), quetiapine fumarate (commercially available as Seroquel®), doxepin hydrochloride (commercially available as Sinequan®), atomoxetine hydrochloride (commercially available as Strattera®), trimipramine maleate (commercially available as Surmontil®), olanzapine/fluoxetine hydrochloride (commercially available as Symbyax®), imipramine hydrochloride (commercially available as Tofranil®), protriptyline hydrochloride (commercially available as Vivactil®), bupropion hydrochloride (commercially available as Wellbutrin®, Wellbutrin SR®, and Wellbutrin XR®), and olanzapine (commercially available as Zyprexa®).

Actives useful herein may also include uric acid reduction treatment, including allopurinol (commercially available as Zyloprim®); seizure medications, including gabapentin (commercially available as Neurontin®), ethotoin (commercially available as Peganone®), vigabatrin (commercially available as Sabril®), and topiramate (commercially available as Topamax®); treatment for shingles, such as zoster vaccine live (commercially available as Zostavax®); skin care medications, including calcipotriene (commercially available as Dovonex®), ustekinumab (commercially available as Stelara®), televancin (commercially available as Vibativ®), isotretinoin (commercially available as Accutane®), hydrocortisone/iodoquinol (commercially available as Alcortin®), sulfacetamide sodium/sulfur (commercially available as Avar®), azelaic acid (commercially available as Azelex®, Finacea®), benzoyl peroxide (commercially available as Desquam-E®), adapalene (commercially available as Differing), fluorouracil (commercially available as Efudex®), pimecrolimus (commercially available as Elidel®), topical erythromycin (commercially available as A/T/S®, Erycette®, T-Stat®), hydrocortisone (commercially available as Cetacort®, Hytone®, Nutracort®), metronidazole (commercially available as MetroGel®), doxycycline (commercially available as Oracea®), tretinoin (commercially available as Retin-A® and Renova®), mequinol/tretinoin (commercially available as Solage®), acitretin (commercially available as Soriatane®), calcipotriene hydrate/betamethasone dipropionate (commercially available as Taclonex®), tazarotene (commercially available as Tazorac®), fluocinonide (commercially available as Vanos®), desonide (commercially available as Verdeso®), miconazole nitrate/Zinc oxide (commercially available as Vusion®), ketoconazole (commercially available as Xolegel®), and efalizumab (commercially available as Raptiva®).

Other actives useful herein may include Sleep disorder medications, including zaleplon (available as Sonata®), eszopiclone (available as Lunesta®), zolpidem tartrate (commercially available as Ambien®, Ambien CR®, Edluar®), lorazepam (commercially available as Ativan®), flurazepam hydrochloride (commercially available as Dalmane®), triazolam (commercially available as Halcion®), clonazepam (commercially available as Klonopin®), barbituates, such as Phenobarbital®), Modafinil (commercially available as Provigil®), temazepam (commercially available as Restoril®), ramelteon (commercially available as Rozerem®), clorazepate dipotassium (commercially available as Tranxene®), diazepam (commercially available as Valium®), quazepam (commercially available as Doral®), and estazolam (commercially available as ProSom®); smoking cessation medications, such as varenicline (commercially available as Chantix®), nicotine, such as Nicotrol®, and bupropion hydrochloride (commercially available as Zyban®); and steroids, including alclometasone dipropionate (commercially available as Aclovate®), betamethasone dipropionate (commercially available as Diprolene®), mometasone furoate (commercially available as Elocon®), fluticasone (commercially available as Flonase®, Flovent®, Flovent Diskus®, Flovent Rotadisk®), fluocinonide (commercially available as Lidex®), mometasone furoate monohydrate (commercially available as Nasonex®), desoximetasone (commercially available as Topicort®), clotrimazole/betamethasone dipropionate (commercially available as Lotrisone®), prednisolone acetate (commercially available as Pred Forte®, Prednisone®, Budesonide Pulmicort®, Rhinocort Aqua®), prednisolone sodium phosphate (commercially available as Pediapred®), desonide (commercially available as Tridesilon®), and halobetasol propionate (commercially available as Ultravate®).

Compositions of the present invention may further include actives useful for thyroid disease treatment, such as hormones TC and TD (commercially available as Armour Thyroid®); potassium deficiency treatment, including potassium chloride (commercially available as Micro-K®); triglycerides regulators, including omega-3-acid ethyl esters (commercially available as Omacor®); urinary medication, such as phenazopyridine hydrochloride (commercially available as Pyridium®) and methenamine, methylene blue/phenyl salicylate/benzoic acid/atropine sulfate/hyoscyamine (commercially available as Urised®); prenatal vitamins (commercially available as Advanced Natalcare®, Materna®, Natalins®, Prenate Advance®); weight control medication, including orlistat (commercially available as Xenical®) and sibutramine hydrochloride (commercially available as Meridia®).

The popular H2-antagonists which are contemplated for use herein include cimetidine, ranitidine hydrochloride, famotidine, nizatidien, ebrotidine, mifentidine, roxatidine, pisatidine and aceroxatidine.

The active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

Examples of specific actives include but are not limited to 16-alpha fluorocstradiol, 16-alpha-gitoxin, 16-epiestriol, 17 alpha dihydroequilenin, 17 alpha estradiol, 17 beta estradiol, 17 hydroxy progesterone, lalpha-hydroxyvitamin D2,1-dodecpyrrolidinone, 20-epi-1,25 dihydroxyvitamin D3, 22-oxacalcitriol, 2CVV, 2′-nor-cGMP, 3-isobutyl GABA, 5-ethynyluracil, 6-FUDCA, 7-methoxytacrine, Abamectin, abanoquil, abecarnil, abiraterone, Ablukast, Ablukast Sodium, Acadesine, acamprosate, Acarbose, Acebutolol, Acecainide Hydrochloride, Aceclidine, aceclofenae, Acedapsone, Aceglutamide Aluminum, Acemannan, Acetaminophen, Acetazolamide, Acetohexamide, Acetohydroxamic Acid, acetomepregenol, Acetophenazine Maleate, Acetosulfone Sodium, Acetylcholine Chloride, Acetylcysteine, acetyl-L-carnitine, acetylmethadol, Acifran, acipimox, acitemate, Acitretin, Acivicin, Aclarubicin, aclatonium, Acodazole Hydrochloride, aconiazide, Acrisorcin, Acrivastine, Acronine, Actisomide, Actodigin, Acyclovir, acylfulvene, adafenoxate, adapalene, Adapalene, adatanserin, Adatanserin Hydrochloride, adecypenol, adecypenol, Adefovir, adelmidrol, ademetionine, Adenosine, Adinazolam, Adipheinine Hydrochloride, adiposin, Adozelesin, adrafinil, Adrenalone, airbutamine, alacepril, Alamecin, Alanine, Alaproclate, alaptide, Albendazole, albolabrin, Albuterol, Albutoin, Alclofenae, Alclometasone Dipropionate, Alcloxa, aldecalmycin, Aldesleukin, Aldioxa, Alendronate Sodium, alendronic acid, alentemol, Alentemol Hydrobromide, Aletamine Hydrochloride, Aleuronium Chloride, Alexidine, alfacalcidol, Alfentanil Hydrochloride, alfuzosin, Algestone Acetonide, alglucerase, Aliflurane, alinastine, Alipamide, Allantoin, Allobarbital, Allopurinol, ALL-TK antagonists, Alogliptin, Alonimid, alosetron, Alosetron Hydrochloride, Alovudine, Alpertine, Alpha Amylase, alpha idosone, Alpidem, Alprazolam, Alprenolol Hydrochloride, Alprenoxime Hydrochloride, Alprostadil, Alrestatin Sodium, Altanserin Tartrate, Alteplase, Althiazide, Altretamine, altromycin B, Alverinc Citrate, Alvircept Sudotox, Amadinone Acetate, Amantadine Hydrochloride, ambamustine, Ambomycin, Ambruticin, Ambuphylline, Ambuside, Amcinafal, Amcinonide, Amdinocillin, Amdinocillin Pivoxil, Amedalin Hydrochloride, amelometasone, Ameltolide, Amesergide, Ametantrone Acetate, amezinium metilsulfate, amfebutamone, Amfenac Sodium, Amflutizole, Amicycline, Amidephrine Mesylate, amidox, Amifloxacin, amifostine, Amikacin, Amiloride Hydrochloride, Aminacrine Hydrochloride, Aminobenzoate Potassium, Aminobenzoate Sodium, Aminocaproic Acid, Aminoglutethimide, Aminohippurate Sodium, aminolevulinic acid, Aminophylline, A minorex, Aminosalicylate sodium, Aminosalicylic acid, Amiodarone, Amiprilose Hydrochloride, Amiquinsin Hydrochloride, amisulpride, Amitraz, Amitriptyline Hydrochloride, Amlexanox, amlodipine, Amobarbital Sodium, Amodiaquine, Amodiaquine Hydrochloride, Amorolfine, Amoxapine, Amoxicillin, Amphecloral, Amphetamine Sulfate, Amphomycin, Amphotericin B, Ampicillin, ampiroxicam, Ampyzine Sulfate, Amquinate, Amrinone, amrinone, amrubicin, Amsacrine, amylin, amythiamicin, Anagestone Acetate, anagrelide, Anakinra, ananain, anaritide, Anaritide Acetate, Anastrozole, Anazolene Sodium, Ancrod, andrographolide, Androstenedione, angiogenesis inhibitors, Angiotensin Amide, Anidoxime, Anileridine, Anilopam Hydrochloride, Aniracetam, Anirolac, Anisotropine Methylbromide, Anistreplase, Anitrazafen, anordrin, antagonist D, antagonist G, antarelix, Antazoline Phosphate, Anthelmycin, Anthralin, Anthramycin, antiandrogen, Acedapsone, Felbamate, antiestrogen, antineoplaston, Antipyrine, antisense oligonucleotides, apadoline, apafant, Apalcillin Sodium, apaxifylline, Apazone, aphidicolin glycinate, Apixifylline, Apomorphine Hydrochloride, apraclonidine, Apraclonidine Hydrochloride, Apramycin, Aprindine, Aprindine Hydrochloride, aprosulate sodium, Aprotinin, Aptazapine Maleate, aptiganel, apurinic acid, apurinic acid, aranidipine, Aranotin, Arbaprostil, arbekicin, arbidol, Arbutamine Hydrochloride, Arclofenin, Ardeparin Sodium, argatroban, Arginine, Argipressin Tannate, Arildone, aripiprazol, arotinolol, Arpinocid, Arteflene, Artilide Fumarate, asimadoline, aspalatone, Asparaginase, Asparic Acid, Aspartocin, asperfuran, Aspirin, aspoxicillin, Asprelin, Astemizole, Astromicin Sulfate, asulacrine, atamestane, Atenolol, atevirdine, Atipamezole, Atiprosin Maleate, Atolide, Atorvastatin Calcium, Atosiban, Atovaquone, atpenin B, Atracurium Besylate, atrimustine, atrinositol, Atropine, Auranofin, aureobasidin A, Aurothioglucose, Avilamycin, Avoparcin, Avridine, Axid, axinastatin 1, axinastatin 2, axinastatin 3, Azabon, Azacitidinie, Azaclorzine Hydrochloride, Azaconazole, azadirachtine, Azalanstat Dihydrochloride, Azaloxan Fumarate, Azanator Maleate, Azanidazole, Azaperone, Azaribine, Azaserine, azasetron, Azatadine Maleate, Azathioprine, Azathioprine Sodium, azatoxin, azatyrosine, azelaic acid, azelastine, azelnidipine, Azepindole, Azetepa, azimilide, Azithromycin, Azlocillin, Azolimine, Azosemide, Azotomycin, Aztreonam, Azumolene Sodium, Bacampicillin Hydrochloride, baccatin III, Bacitracin, Baclofen, bacoside A, bacoside B, bactobolamine, balanol, balazipone, balhimycin, balofloxacin, balsalazide, Bambermycins, bambuterol, Bamethan Sulfate, Bamifylline Hydrochloride, Bamidazole, baohuoside 1, Barmastine, barnidipine, Basifungin, Batanopride Hydrochloride, batebulast, Batelapine Maleate, Batimastat, beauvericin, Becanthone Hydrochloride, becaplermin, becliconazole, Beclomethasone Dipropionate, befloxatone, Beinserazide, Belfosdil, Belladonna, Beloxamide, Bemesetron, Bemitradine, Bemoradan, Benapryzine Hydrochloride, Benazepril Hydrochloride, Benazeprilat, Bendacalol Mesylate, Bendazac, Bendroflumethiazide, benflumetol, benidipine, Benorterone, Benoxaprofen, Benoxaprofen, Benoxinate Hydrochloride, Benperidol, Bentazepam, Bentiromide, Benurestat, Benzbromarone, Benzethonium Chloride, Benzetimide Hydrochloride, Benzilonium Bromide, Benzindopyrine Hydrochloride, benzisoxazole, Benzocaine, benzochlorins, Benzoctamine Hydrochloride, Benzodepa, benzoidazoxan, Benzonatate, Benzoyl Peroxide, Benzoylpas Calcium, benzoylstaurosporine, Benzquinamide, Benzthiazide, benztropine, Benztropine Mesylate, Benzydamine Hydrochloride, Benzylpenicilloyl Polylysine, bepridil, Bepridil Hydrochloride, Beractant, Beraprost, Berefrine, berlafenone, bertosamil, Berythromycin, besipirdine, beta-alethine, betaclamycin B, Betamethasone, betamipron, betaxolol, Betaxolol Hydrochloride, Bethanechol Chloride, Bethanidine Sulfate, betulinic acid, bevantolol, Bevantolol Hydrochloride, Bezafibrate, bFGF inhibitor, Bialamicol Hydrochloride, Biapenem, Bicalutamide, Bicifadine Hydrochloride, Biclodil Hydrochloride, Bidisomide, bifemelane, Bifonazole, bimakalim, bimithil, Bindarit, Biniramycin, binospirone, bioxalomycin alpha2, Bipenamol Hydrochloride, Biperiden, Biphenamine Hydrochloride, biriperone, bisantrene, bisaramil, bisaziridinylspermine, bis-benzimidazole A, bis-benzimidazole B, bisnafide, Bisobrin Lactate, Bisoprolol, Bispyrithione Magsulfex, bistramide D, bistramide K, bistratene A, Bithionolate Sodium, Bitolterol Mesylate, Bivalirudin, Bizelesin, Bleomycin Sulfate, Bolandiol Dipropionate, Bolasterone, Boldenone Undecylenate, boldine, Bolenol, Bolmantalate, bopindolol, Bosentan, Boxidine, brefeldin, breflate, Brequinar Sodium, Bretazenil, Bretylium Tosylate, Brifentanil Hydrochloride, brimonidine, Brinolase, Brocresine, Brocrinat, Brofoxine, Bromadoline Maleate, Bromazepam, Bromchlorenone, Bromelains, bromfenac, Brominidione, Bromocriptine, Bromodiphenhydramine Hydrochloride, Bromoxamide, Bromperidol, Bromperidol Decanoate, Brompheniramine Maleate, Broperamole, Bropirimine, Brotizolam, Bucainide Maleate, bucindolol, Buclizine Hydrochloride, Bucromarone, Budesonide, budipine, budotitane, Buformin, Bumetamide, Bunaprolast, bunazosin, Bunolol Hydrochloride, Bupicomide, Bupivacaine Hydrochloride, Buprenorphine Hydrochloride, Bupropion Hydrochloride, Buramate, Buserelin Acetate, Buspirone Hydrochloride, Busulfan, Butabarbital, Butacetin, Butaclamol Hydrochloride, Butalbital, Butamben, Butamirate Citrate, Butaperazine, Butaprost, Butedronate Tetrasodium, butenafine, Buterizine, buthionine sulfoximine, Butikacin, Butilfenin, Butirosin Sulfate, Butixirate, butixocort propionate, Butoconazole Nitrate, Butonate, Butopamine, Butoprozine Hydrochloride, Butorphanol, Butoxamine Hydrochloride, Butriptyline Hydrochloride, Cactinomycin, Cadexomer Iodine, Caffeine, calanolide A, Calcifediol, Calcipotriene, calcipotriol, Calcitonin, Calcitriol, Calcium Undecylenate, calphostin C, Calusterone, Cambendazole, camonagrel, camptothecin derivatives, canagliflozin, canarypox IL-2, candesartan, Candicidin, candoxatril, candoxatrilat, Caniglibose, Canrenoate Potassium, Canrenone, capecitabine, Capobenate Sodium, Capobenic Acid, Capreomycin Sulfate, capromab, capsaicin, Captopril, Capuride, Caracemide, Carbachol, Carbadox, Carbamazepine, Carbamide Peroxide, Carbantel Lauryl Sulfate, Carbaspirin Calcium, Carbazeran, carbazomycin C, Carbenicillin Potassium, Carbenoxolone Sodium, Carbetimer, carbetocin, Carbidopa, Carbidopa-Levodopa, Carbinoxamine Maleate, Carbiphene Hydrochloride, Carbocloral, Carbocysteine, Carbol-Fuchsin, Carboplatin, Carboprost, carbovir, carboxamide-amino-triazo-le, carboxyamidotriazole, carboxymethylated beta-1,3-glucan, Carbuterol Hydrochloride, CaRest M3, Carfentanil Citrate, Carisoprodol, Carmantadine, Carmustine, CARN 700, Camidazole, Caroxazone, carperitide, Carphenazine Maleate, Carprofen, Carsatrin Succinate, Cartazolate, carteolol, Carteolol Hydrochloride, cartilage derived inhibitor, Carubicin Hydrochloride, Carumonam Sodium, carvedilol, carvotroline, Carvotroline Hydrochloride, carzelesin, casein kinase inhibitors (ICOS), castanospermine, caurumonam, cebaracetam, cecropin B, Cedefingol, Cefaclor, Cefadroxil, Cefamandole, Cefaparole, Cefatrizine, Cefazaflur Sodium, Cefazolin, Cefbuperazone, cefcapene pivoxil, cefdaloxime pentexil tosilate, Cefdinir, cefditoren pivoxil, Cefepime, cefetamet, Cefetecol, cefixime, cefluprenam, Cefinenoxime Hydrochloride, Cefinetazole, cefminlox, cefodizime, Cefonicid Sodium, Cefoperazone Sodium, Ceforamide, cefoselis, Cefotaxime Sodium, Cefotetan, cefotiam, Cefoxitin, cefozopran, cefpimizole, Cefpiramide, cefpirome, cefpodoxime proxetil, cefprozil, Cefroxadine, cefsulodin, Ceftazidime, cefteram, ceftibuten, Ceftizoxime Sodium, ceftriaxone, Cefuroxime, celastrol, celikalim, celiprolol, cepacidiine A, Cephacetrile Sodium, Cephalexin, Cephaloglycin, Cephaloridine, Cephalothin Sodium, Cephapirin Sodium, Cephradine, cericlamine, cerivastatin, Ceronapril, certoparin sodium, Ceruletide, Cetaben Sodium, Cetalkonium Chloride, Cetamolol Hydrochloride, cetiedil, cetirizine, Cetophenicol, Cetraxate Hydrochloride, cetrorelix, Cetylpyridinium Chloride, Chenodiol, Chlophedianol Hydrochloride, Chloral Betaine, Chlorambucil, Chloramphenicol, Chlordantoin, Chlordiazepoxide, Chlorhexidine Gluconate, chlorins, Chlormadinone Acetate, chloroorienticin A, Chloroprocaine Hydrochloride, Chloropropamide, Chloroquine, chloroquinoxaline sulfonamide, Chlorothiazide, Chlorotrianisene, Chloroxine, Chloroxylenol, Chlorphenesin Carbamate, Chlorpheniramine Maleate, Chlorpromazine, Chlorpropamide, Chlorprothixene, Chlortetracycline Bisulfate, Chlorthalidone, Chlorzoxazone, Cholestyramine Resin, Chromonar Hydrochloride, cibenzoline, cicaprost, Ciclafrine Hydrochloride, Ciclazindol, ciclesonide, cicletanine, Ciclopirox, Cicloprofen, cicloprolol, Cidofovir, Cidoxepin Hydrochloride, Cifenline, Ciglitazone, Ciladopa Hydrochloride, cilansetron, Cilastatin Sodium, Cilazapril, cilnidipine, Cilobamine Mesylate, cilobradine, Cilofungin, cilostazol, Cimaterol, Cimetidine, cimetropium bromide, Cinalukast, Cinanserin Hydrochloride, Cinepazet Maleate, Cinflumide, Cingestol, cinitapride, Cinnamedrine, Cinnarizine, cinolazepam, Cinoxacin, Cinperene, Cinromide, Cintazone, Cintriamide, Cioteronel, Cipamfylline, Ciprefadol Succinate, Ciprocinonide, Ciprofibrate, Ciprofloxacin, ciprostene, Ciramadol, Cirolemycin, cisapride, cisatracurium besilate, Cisconazole, Cisplatin, cis-porphyrin, cistinexine, citalopram, Citenamide, citicoline, citreamicin alpha, cladribine, Clamoxyquin Hydrochloride, Clarithromycin, clausenamide, Clavulanate Potassium, Clazolam, Clazolimine, clebopride, Clemastine, Clentiazem Maleate, Clidinium Bromide, clinafloxacin, Clindamycin, Clioquinol, Clioxamide, Cliprofen, clobazam, Clobetasol Propionate, Clobetasone Butyrate, Clocortolone Acetate, Clodanolene, Clodazon Hydrochloride, clodronic acid, Clofazimine, Clofibrate, Clofilium Phosphate, Clogestone Acetate, Clomacran Phosphate, Clomegestone Acetate, Clometherone, clomethiazole, clomifene analogues, Clominorex, Clomiphene, Clomipramine Hydrochloride, Clonazepam, Clonidine, Clonitrate, Clonixeril, Clonixin, Clopamide, Clopenthixol, Cloperidone Hydrochloride, clopidogrel, Clopimozide, Clopipazan Mesylate, Clopirac, Cloprednol, Cloprostenol Sodium, Clorazepate Dipotassium, Clorethate, Clorexolone, Cloroperone Hydrochloride, Clorprenaline Hydrochloride, Clorsulon, Clortermine Hydrochloride, Closantel, Closiramine Aceturate, Clothiapine, Clothixamide Maleate Cloticasone Propionate, Clotrimazole, Cloxacillin Benzathine, Cloxyquin, Clozapine, Cocaine, Coccidioidin, Codeine, Codoxime, Colchicine, colestimide, Colestipol Hydrochloride, Colestolone, Colforsin, Colfosceril Palmitate, Colistimethate Sodium, Colistin Sulfate, collismycin A, collismycin B, Colterol Mesylate, combretastatin A4, combretastatin analogue, complestatin, conagenin, Conorphone Hydrochloride, contignasterol, contortrostatin, Cormethasone Acetate, Corticorelin Ovine Triflutate, Corticotropin, Cortisone Acetate, Cortivazol, Cortodoxone, cosalane, costatolide, Cosyntropin, cotinine, Coumadin, Coumermycin, crambescidin 816, Crilvastatin, crisnatol, Cromitrile Sodium, Cromolyn Sodium, Crotamiton, cryptophycin 8, cucumariosid, Cuprimyxin, curacin A, curdlan sulfate, curiosin, Cyclacillin, Cyclazocine, cyclazosin, cyclic HPMPC, Cyclindole, Cycliramine Maleate, Cyclizine, Cyclobendazole, cyclobenzaprine, cyclobut A, cyclobut G, cyclocapron, Cycloguanil Pamoate, Cycloheximide, cyclopentanthraquinones, Cyclopenthiazide, Cyclopentolate Hydrochloride, Cyclophenazine Hydrochloride, Cyclophosphamide, cycloplatam, Cyclopropane, Cycloserine, cyclosin, Cyclosporine, cyclothialidine, Cyclothiazide, cyclothiazomycin, Cyheptamide, cypemycin, Cypenamine Hydrochloride, Cyprazepam, Cyproheptadine Hydrochloride, Cyprolidol Hydrochloride, cyproterone, Cyproximide, Cysteamine, Cysteine Hydrochloride, Cystine, Cytarabine, Cytarabine Hydrochloride, cytarabine ocfosfate, cytochalasin B, cytolytic factor, cytostatin, Dacarbazine, dacliximab, dactimicin, Dactinomycin, daidzein, Daledalin Tosylate, dalfopristin, Dalteparin Sodium, Daltroban, Dalvastatin, danaparoid, Danazol, Dantrolene, dapagliflozin, daphlnodorin A, dapiprazole, dapitant, Dapoxetine Hydrochloride, Dapsone, Daptomycin, Darglitazone Sodium, darifenacin, darlucin A, Darodipine, darsidomine, Daunorubicin Hydrochloride, Dazadrol Maleate, Dazepinil Hydrochloride, Dazmegrel, Dazopride Fumarate, Dazoxiben Hydrochloride, Debrisoquin Sulfate, Decitabine, deferiprone, deflazacort, Dehydrocholic Acid, dehydrodidemnin B, Dehydroepiandrosterone, delapril, Delapril Hydrochloride, Delavirdine Mesylate, delequamine, delfaprazine, Delmadinone Acetate, delmopinol, delphinidin, Demecarium Bromide, Demeclocycline, Demecycline, Demoxepam, Denofungin, deoxypyridinoline, Depakote, deprodone, Deprostil, depsidomycin, deramciclane, dermatan sulfate, Desciclovir, Descinolone Acetonide, Desflurane, Desipramine Hydrochloride, desirudin, Deslanoside, deslorelin, desmopressin, desogestrel, Desonide, Desoximetasone, desoxoamiodarone, Desoxycorticosterone Acetate, detajmium bitartrate, Deterenol Hydrochloride, Detirelix Acetate, Devazepide, Dexamethasone, Dexamisole, Dexbrompheniramine Maleate, Dexchlorpheniramine Maleate, Dexclamol Hydrochloride, Dexetimide, Dexfenfluramine Hydrochloride, dexifosfamide, Deximafen, Dexivacaine, dexketoprofen, dexloxiglumide, Dexmedetomidine, Dexormaplatin, Dexoxadrol Hydrochloride, Dexpanthenol, Dexpemedolac, Dexpropranolol Hydrochloride, Dexrazoxane, dexsotalol, dextrin 2-sulphate, Dextroamphetamine, Dextromethorphan, Dextrorphan Hydrochloride, Dextrothyroxine Sodium, dexverapamil, Dezaguanine, dezinamide, dezocine, Diacetolol Hydrochloride, Diamocaine Cyclamate, Diapamide, Diatrizoate Meglumine, Diatrizoic Acid, Diaveridine, Diazepam, Diaziquone, Diazoxide, Dibenzepin Hydrochloride, Dibenzothiophene, Dibucaine, Dichliorvos, Dichloralphenazone, Dichlorphenamide, Dicirenone, Diclofenac Sodium, Dicloxacillin, dicranin, Dicumarol, Dicyclomine Hydrochloride, Didanosine, didemnin B, didox, Dienestrol, dienogest, Diethylcarbamazine Citrate, diethylhomospermine, diethylnorspermine, Diethylpropion Hydrochloride, Diethylstilbestrol, Difenoximide Hydrochloride, Difenoxin, Diflorasone Diacetate, Difloxacin Hydrochloride, Difluanine Hydrochloride, Diflucortolone, Diflumidone Sodium, Diflunisal, Difluprednate, Diftalone, Digitalis, Digitoxin, Digoxin, Dihexyverine Hydrochloride, dihydrexidine, dihydro-5-azacytidine, Dihydrocodeine Bitartrate, Dihydroergotamine Mesylate, Dihydroestosterone, Dihydrostreptomycin Sulfate, Dihydrotachysterol, dihydrotaxol, 9-, Dilantin, Dilevalol Hydrochloride, Diltiazem Hydrochloride, Dimefadane, Dimefline Hydrochloride, Dimenhydrinate, Dimercaprol, Dimethadione, Dimethindene Maleate, Dimethisterone, dimethyl prostaglandin A1, Dimethyl Sulfoxide, dimethylhomospermine, dimiracetam, Dimoxamine Hydrochloride, Dinoprost, Dinoprostone, Dioxadrol Hydrochloride, dioxamycin, Diphenhydramine Citrate, Diphenidol, Diphenoxylate Hydrochloride, diphenyl spiromustine, Dipivefin Hydrochloride, Dipivefrin, dipliencyprone, diprafenone, dipropylnorspermine, Dipyridamole, Dipyrithione, Dipyrone, dirithromycin, discodermolide, Disobutamide, Disofenin, Disopyramide, Disoxaril, disulfiram, Ditekiren, Divalproex Sodium, Dizocilpine Maleate, Dobutamine, docarpamine, Docebenone, Docetaxel, Doconazole, docosanol, dofetilide, dolasetron, Ebastine, ebiratide, ebrotidine, ebselen, ecabapide, ecabet, ecadotril, ecdisteron, echicetin, echistatin, Echothiophate Iodide, Eclanamine Maleate, Eclazolast, ecomustine, Econazole, ecteinascidin 722, edaravone, Edatrexate, edelfosine, Edifolone Acetate, edobacomab, Edoxudine, edrecolomab, Edrophonium Chloride, edroxyprogesteone Acetate, efegatran, eflornithine, efonidipine, egualcen, Elantrine, eleatonin, elemene, eletriptan, elgodipine, eliprodil, Elsamitrucin, eltenae, Elucaine, emalkalim, emedastine, Emetine Hydrochloride, emiglitate, Emilium Tosylate, emitefur, emoctakin, empagliflozin, Enadoline Hydrochloride, enalapril, Enalaprilat, Enalkiren, enazadrem, Encyprate, Endralazine Mesylate, Endrysone, Enflurane, englitazone, Enilconazole, Enisoprost, Enlimomab, Enloplatin, Enofelast, Enolicam Sodium, Enoxacin, enoxacin, enoxaparin sodium, Enoxaparin Sodium, Enoximone, Enpiroline Phosphate, Enprofylline, Enpromate, entacapone, enterostatin, Enviradene, Enviroxime, Ephedrine, Epicillin, Epimestrol, Epinephrine, Epinephryl Borate, Epipropidine, Epirizole, epirubicin, Epitetracycline Hydrochloride, Epithiazide, Epoetin Alfa, Epoetin Beta, Epoprostenol, Epoprostenol Sodium, epoxymexrenone, epristeride, Eprosartan, eptastigmine, equilenin, Equilin, Erbulozole, erdosteine, Ergoloid Mesylates, Ergonovine Maleate, Ergotamine Tartrate, ersentilide, Ersofermin, erythritol, Erythrityl Tetranitrate, Erythromycin, Esmolol Hydrochloride, Esorubicin Hydrochloride, Esproquin Hydrochloride, Estazolam, Estradiol, Estramustine, estramustine analogue, Estrazinol Hydrobromide, Estriol, Estrofurate, estrogen agonists, estrogen antagonists, Estrogens, Conjugated Estrogens, Esterified Estrone, Estropipate, esuprone, Etafedrine Hydrochloride, Etanidazole, etanterol, Etarotene, Etazolate Hydrochloride, Eterobarb, ethacizin, Ethacrynate Sodium, Ethacrynic Acid, Ethambutol Hydrochloride, Ethamivan, Ethanolamine Oleate, Ethehlorvynol, Ether, Ethinyl estradiol, Ethiodized Oil, Ethionamide, Ethonam Nitrate, Ethopropazine Hydrochloride, Ethosuximide, Ethotoin, Ethoxazene Hydrochloride, Ethybenztropine, Ethyl Chloride, Ethyl Dibunate, Ethylestrenol, Ethyndiol, Ethynerone, Ethynodiol Diacetate, Etibendazole, Etidocaine, Etidronate Disodium, Etidronic Acid, Etifenin, Etintidine Hydrochloride, etizolam, Etodolac, Etofenamate, Etoformin Hydrochloride, Etomidate, Etonogestrel, Etoperidone Hydrochloride, Etoposide, Etoprine, Etoxadrol Hydrochloride, Etozolin, etrabamine, Etretinate, Etryptamine Acetate, Eucatropine Hydrochloride, Eugenol, Euprocin Hydrochloride, eveminomicin, Exametazime, examorelin, Exaprolol Hydrochloride, exemestane, fadrozole, faeriefungin, Famciclovir, Famotidine, Fampridine, fantofarone, Fantridone Hydrochloride, faropenem, fasidotril, fasudil, fazarabine, fedotozine, felbamate, Felbinac, Felodipine, Felypressin, Fenalamide, Fenamole, Fenbendazole, Fenbufen, Fencibutirol, Fenclofenac, Fenclonine, Fenclorac, Fendosal, Fenestrel, Fenethylline Hydrochloride, Fenfluramine Hydrochloride, Fengabine, Fenimide, Fenisorex, Fenmetozole Hydrochloride, Fenmetramide, Fenobam, Fenoctimine Sulfate, fenofibrate, fenoldopam, Fenoprofen, Fenoterol, Fenpipalone, Fenprinast Hydrochloride, Fenprostalene, Fenquizone, fenretinide, fenspiride, Fentanyl Citrate, Fentiazac, Fenticlor, fenticonazole, Fenyripol Hydrochloride, fepradinol, ferpifosate sodium, ferristene, ferrixan, Ferrous Sulfate, Dried, Ferumoxides, ferumoxsil, Fetoxylate Hydrochloride, fexofenadine, Fezolamine Fumarate, Fiacitabine, Fialuridine, Fibrinogen 1 125, filgrastim, Filipin, finasteride, Flavodilol Maleate, flavopiridol, Flavoxate Hydrochloride, Flazalone, flecainide, flerobuterol, Fleroxacin, flesinoxan, Flestolol Sulfate, Fletazepam, flezelastine, flobufen, Floctafenine, flomoxef, Flordipine, florfenicol, florifenine, flosatidil, Flosequinan, Floxacillin, Floxuridine, fluasterone, Fluazacort, Flubanilate Hydrochloride, Flubendazole, Flucindole, Flucloronide, Fluconazole, Flucytosine, Fludalanine, Fludarabine Phosphate, Fludazonium Chloride, Fludeoxyglucose F 18, Fludorex, Fludrocortisone Acetate, Flufenamic Acid, Flufenisal, Flumazenil, flumecinol, Flumequine, Flumeridone, Flumethasone, Flumetramide, Flumezapine, Fluminorex, Flumizole, Flumoxonide, flunarizine, Flunidazole, Flunisolide, Flunitrazepam, Flunixin, fluocalcitriol, Fluocinolone Acetonide, Fluocinonide, Fluocortin Butyl, Fluocortolone, Fluorescein, fluorodaunorunicin hydrochloride, Fluorodopa F 18, Fluorometholone, Fluorouracil, Fluotracen Hydrochloride, Fluoxetine, Fluoxymesterone, fluparoxan, Fluperamide, Fluperolone Acetate, Fluphenazine Decanoate, flupirtine, Fluprednisolone, Fluproquazone, Fluprostenol Sodium, Fluquazone, Fluradoline Hydrochloride, Flurandrenolide, Flurazepam Hydrochloride, Flurbiprofen, Fluretofen, flurithromycin, Flurocitabine, Flurofamide, Flurogestone Acetate, Flurothyl, Fluroxene, Fluspiperone, Fluspirilene, Fluticasone Propionate, flutrimazole, Flutroline, fluvastatin, Fluvastatin Sodium, fluvoxamine, Fluzinamide, Folic Acid, Follicle regulatory protein, Folliculostatin, Fomepizole, Fonazine Mesylate, forasartan, forfenimex, forfenirmex, formestane, Formocortal, formoterol, Fosarilate, Fosazepam, Foscarnet Sodium, fosfomycin, Fosfonet Sodium, fosinopril, Fosinoprilat, fosphenyloin, Fosquidone, Fostedil, fostriecin, fotemustine, Fuchsin, Basic, Fumoxicillin, Fungimycin, Furaprofen, Furazolidone, Furazolium Chloride, Furegrelate Sodium, Furobufen, Furodazole, Furosemide, Fusidate Sodium, Fusidic Acid, gabapentin, Gadobenate Dimeglumine, gadobenic acid, gadobutrol, Gadodiamide, gadolinium texaphyrin, Gadopentetate Dimegiumine, gadoteric acid, Gadoteridol, Gadoversetamide, galantamine, galdansetron, Galdansetron Hydrochloride, Gallamine Triethiodide, gallium nitrate, gallopamil, galocitabine, Gamfexine, gamolenic acid, Ganciclovir, ganirelix, gelatinase inhibitors, Gemcadiol, Gemcitabine, Gemeprost, Gemfibrozil, Gentamicin Sulfate, Gentian Violet, gepirone, Gestaclone, Gestodene, Gestonorone Caproate, Gestrinone, Gevotroline Hydrochloride, girisopam, glaspimod, glaucocalyxin A, Glemanserin, Gliamilide, Glibornuride, Glicetanile Sodium, Gliflumide, Glimepiride, Glipizide, Gloximonam, Glucagon, glutapyrone, glutathione inhibitors, Glutethimide, Glyburide, glycopine, glycopril, Glycopyrrolate, Glyhexamide, Glymidine Sodium, Glyoctamide, Glyparamide, Gold Au 198, Gonadoctrinins, Gonadorelin, Gonadotropins, Goserelin, Gramicidin, Granisetron, grepafloxacin, Griseofulvin, Guaiapate, Guaithylline, Guanabenz, Guanabenz Acetate, Guanadrel Sulfate, Guancydine, Guanethidine Monosulfate, Guanfacine Hydrochloride, Guanisoquin Sulfate, Guanoclor Sulfate, Guanoctine Hydrochloride, Guanoxabenz, Guanoxan Sulfate, Guanoxyfen Sulfate, Gusperimus Trihydrochloride, Halazepam, Halcinonide, halichondrin B, Halobetasol Propionate, halofantrine, Halofantrine Hydrochloride, Halofenate, Halofuginone Hydrobromide, halomon, Halopemide, Haloperidol, halopredone, Haloprogesterone, Haloprogin, Halothane, Halquinols, Hamycin, Han memopausal gonadotropins, hatomamicin, hatomarubigin A, hatomarubigin B, hatomarubigin C, hatomarubigin D, Heparin Sodium, hepsulfam, heregulin, Hetacillin, Heteronium Bromide, Hexachlorophene: Hydrogen Peroxide, Hexafluorenium Bromide, hexamethylene bisacetamide, Hexedine, Hexobendine, Hexoprenaline Sulfate, Hexylresorcinol, Histamine Phosphate, Histidine, Histoplasmin, Histrelin, Homatropine Hydrobromide, Hoquizil Hydrochloride, Human chorionic gonadotropin, Hycanthone, Hydralazine Hydrochloride, Hydralazine Polistirex, Hydrochlorothiazide, Hydrocodone Bitartrate, Hydrocortisone, Hydroflumethiazide, Hydromorphone Hydrochloride, Hydroxyamphetamine Hydrobromide, Hydroxychloroquine Sulfate, Hydroxyphenamate, Hydroxyprogesterone Caproate, Hydroxyurca, Hydroxyzine Hydrochloride, Hymecromone, Hyoscyamine, hypericin, Ibafloxacin, ibandronic acid, ibogaine, Ibopamine, ibudilast, Ibufenac, Ibuprofen, Ibutilide Fumarate, Icatibant Acetate, Ichthammol, Icotidine, idarubicin, idoxifene, Idoxuridine, idramantone, Iemefloxacin, Iesopitron, Ifetroban, Ifosfamide, Ilepeimide, illimaquinone, ilmofosine, ilomastat, Ilonidap, iloperidone, iloprost, Imafen Hydrochloride, Imazodan Hydrochloride, imidapril, imidazenil, imidazoacridones, Imidecyl Iodine, Imidocarb Hydrochloride, Imidoline Hydrochloride, Imidurea, Imiloxan Hydrochloride, Imipenem, Imipramine Hydrochloride, imiquimod, immunostimulant peptides, Impromidine Hydrochloride, Indacrinone, Indapamide, Indecainide Hydrochloride, Indeloxazine Hydrochloride, Indigotindisulfonate Sodium, indinavir, Indocyanine Green, Indolapril Hydrochloride, Indolidan, indometacin, Indomethacin Sodium, Indoprofen, indoramin, Indorenate Hydrochloride, Indoxole, Indriline Hydrochloride, inocoterone, inogatran, inolimomab, Inositol Niacinate, Insulin, interferons, interleukins, Intrazole, Intriptyline Hydrochloride, iobenguane, Iobenzamic Acid, iobitridol, Iocarmate Meglumine, Iocarmic Acid, Iocetamic Acid, Iodamide, Iodine, Iodipamide Meglumine, Iodixanol, iodoamiloride, Iodoantipyrine I 131, Iodocholesterol I 131, iododoxorubicin, Iodohippurate Sodium I 131, Iodopyracet I 125, Iodoquinol, Iodoxamate Meglumine, Iodoxamie Acid, Ioglicic Acid, Iofetamine Hydrochloride I 123, iofratol, Ioglucol, Ioglucomide, Ioglycamic Acid, Iogulamide, Iohexol, iomeprol, Iomethin I 125, Iopamidol, Iopanoic Acid, iopentol, Iophendylate, Ioprocemic Acid, iopromide, Iopronic Acid, Iopydol, Iopydone, iopyrol, Iosefamic Acid, Ioseric Acid, Iosulamide Meglumine, Iosumetic Acid, Iotasul, Iotetric Acid, Iothalamate Sodium, Iothalamic Acid, iotriside, Iotrolan, Iotroxic Acid, Iotyrosine I 131, Ioversol, Ioxagiate Sodium, Ioxaglate Meglumine, Ioxaglic Acid, ioxilan, Ioxotrizoic Acid, ipazilide, ipenoxazone, ipidacrine, Ipodate Calcium, ipomeanol, 4-, Ipratropium Bromide, ipriflavone, Iprindole, Iprofenin, Ipronidazole, Iproplatin, Iproxamine Hydrochloride, ipsapirone, irbesartan, irinotecan, irloxacin, iroplact, irsogladine, Irtemazole, isalsteine, Isamoxole, isbogrel, Isepamicin, isobengazole, Isobutamben, Isocarboxazid, Isoconazole, Isoetharine, isofloxythepin, Isoflupredone Acetate, Isoflurane, Isoflurophate, isohomohalicondrin B, Isoleucine, Isomazole Hydrochloride, Isomylamine Hydrochloride, Isoniazid, Isopropamide Iodide, Isopropyl Alcohol, isopropyl unoprostone, Isoproterenol Hydrochloride, Isosorbide, Isosorbide Mononitrate, Isotiquimide, Isotretinoin, Isoxepac, Isoxicam, Isoxsuprine Hydrochloride, isradipine, itameline, itasetron, Itazigrel, itopride, Itraconazole, Ivermectin, jasplakinolide, Josamycin, kahalalide F, Kalafungin, Kanamycin Sulfate, Ketamine Hydrochloride, Ketanserin, Ketazocine, Ketazolam, Kethoxal, Ketipramine Fumarate, Ketoconazole, Ketoprofen, Ketorfanol, ketorolac, Ketotifen Fumarate, Kitasamycin, Labetalol Hydrochloride, Lacidipine, lacidipine, lactitol, lactivicin, lacosamide, laennec, lafutidine, lamellarin-N triacetate, lamifiban, Lamivudine, Lamotrigine, lanoconazole, Lanoxin, lanperisone, lanreotide, Lansoprazole, latanoprost, lateritin, laurocapram, Lauryl Isoquinolinium Bromide, Lavoltidine Succinate, lazabemide, Lecimibide, leinamycin, lemildipine, leminoprazole, lenercept, Leniquinsin, lenograstim, Lenperone, lentinan sulfate, leptin, leptolstatin, lercanidipine, Lergotrile, lerisetron, Letimide Hydrochloride, letrazuril, letrozole, Leucine, leucomyzin, Leuprolide Acetate, leuprolide+estrogen+progesterone, leuprorelin, Levamfetamine Succinate, levamisole, Levdobutamine Lactobionate, Leveromakalim, levetiracetam, Leveycloserine, levobetaxolol, levobunolol, levobupivacaine, levocabastine, levocarnitine, Levodopa, levodropropizine, levofloxacin, Levofuraltadone, Levoleucovorin Calcium, Levomethadyl Acetate, Levomethadyl Acetate Hydrochloride, levomoprolol, Levonantradol Hydrochloride, Levonordefrin, Levonorgestrel, Levopropoxyphene Napsylate, Levopropylcillin Potassium, levormeloxifene, Levorphanol Tartrate, levosimendan, levosulpiride, Levothyroxine Sodium, Levoxadrol Hydrochloride, Lexipafant, Lexithromycin, liarozole, Libenzapril, Lidamidine Hydrochloride, Lidocaine, Lidofenin, Lidoflazine, Lifarizine, Lifibrate, Lifibrol, Linarotene, Lincomycin, linear polyamine analogue, Linogliride, Linopirdine, linotroban, linsidomine, lintitript, lintopride, Liothyronine I 125, liothyronine sodium, Liotrix, lirexapride, lisinopril, lissoclinamide 7, Lixazinone Sulfate, lobaplatin, Lobenzarit Sodium, Lobucavir, Lodelaben, Iodoxamide, Lofemizole Hydrochloride, Lofentanil Oxalate, Lofepramine Hydrochloride, Lofexidine Hydrochloride, lombricine, Lomefloxacin, lomerizine, Lometraline Hydrochloride, lometrexol, Lomofungin, Lomoxicam, Lomustine, Lonapalene, lonazolac, lonidamine, Loperamide Hydrochloride, loracarbef, Lorajmine Hydrochloride, loratadine, Lorazepam, Lorbamate, Lorcainide Hydrochloride, Loreclezole, Loreinadol, lorglumide, Lormetazepam, Lornoxicam, lornoxicam, Lortalamine, Lorzafone, losartan, losigamone, losoxantrone, Losulazine Hydrochloride, loteprednol, lovastatin, loviride, Loxapine, Loxoribine, lubeluzole, Lucanthone Hydrochloride, Lufironil, Lurosetron Mesylate, lurtotecan, luteinizing hormone, lurasidone, lutetium, Lutrelin Acetate, luzindole, Lyapolate Sodium, Lycetamine, lydicamycin, Lydimycin, Lynestrenol, Lypressin, Lysine, lysofylline, lysostaphin, lytic peptides, Maduramicin, Mafenide, magainin 2 amide, Magnesium Salicylate, Magnesium Sulfate, magnolol, maitansine, Malethamer, mallotochromene, mallotoj aponin, Malotilate, malotilate, mangafodipir, manidipine, maniwamycin A, Mannitol, mannostatin A, manumycin E, manumycin F, mapinastine, Maprotiline, marimastat, Martek 8708, Martek 92211, Masoprocol, maspin, massetolide, matrilysin inhibitors, Maytansine, Mazapertine Succiniate, Mazindol, Mebendazole, Mebeverine Hydrochloride, Mebrofenin, Mebutamate, Mecamylamine Hydrochloride, Mechlorethamine Hydrochloride, Meclocycline, Meclofenamate Sodium, Mecloqualone, Meclorisone Dibutyrate, Medazepam Hydrochloride, Medorinone, Medrogestone, Medroxalol, Medroxyprogesterone, Medrysone, Meelizine Hydrochloride, Mefenamic Acid, Mefenidil, Mefenorex Hydrochloride, Mefexamide, Mefloquine Hydrochloride, Mefruside, Megalomicin Potassium Phosphate, Megestrol Acetate, Meglumine, Meglutol, Melengestrol Acetate, Melitracen Hydrochloride, Melphalan, Memotine Hydrochloride, Menabitan Hydrochloride, Menoctone, menogaril, Menotropins, Meobentine Sulfate, Mepartricin, Mepenzolate Bromide, Meperidine Hydrochloride, Mephentermine Sulfate, Mephenyloin, Mephobarbital, Mepivacaine Hydrochloride, Meprobamate, Meptazinol Hydrochloride, Mequidox, Meralein Sodium, merbarone, Mercaptopurine, Mercufenol Chloride, Mercury, Ammoniated, Merisoprol Hg 197, Meropenem, Mesalamine, Meseclazone, Mesoridazine, Mesterolone, Mestranol, Mesuprine Hydrochloride, Metalol Hydrochloride, Metaproterenol Polistirex, Metaraminol Bitartrate, Metaxalone, Meteneprost, meterelin, Metformin, Methacholine Chloride, Methacycline, Methadone Hydrochloride, Methadyl Acetate, Methalthiazide, Methamphetamine Hydrochloride, Methaqualone, Methazolamide, Methdilazine, Methenamine, Methenolone Acetate, Methetoin, Methicillin Sodium, Methimazole, methioninase, Methionine, Methisazone, Methixene Hydrochloride, Methocarbamol, Methohexital Sodium, Methopholine, Methotrexate, Methotrimeprazine, methoxatone, Methoxyflurane, Methsuximide, Methyclothiazide, Methyl Palmoxirate, Methylatropine Nitrate, Methylbenzethonium Chloride, Methyldopa, Methyldopate Hydrochloride, Methylene Blue, Methylergonovine Maleate, methylhistamine, R-alpha, methylinosine monophosphate, Methylphenidate Hydrochloride, Methylprednisolone, Methyltestosterone, Methynodiol Diacelate, Methysergide, Methysergide Maleate, Metiamide, Metiapine, Metioprim, metipamide, Metipranolol, Metizoline Hydrochloride, Metkephamid Acetate, metoclopramide, Metocurine Iodide, Metogest, Metolazone, Metopimazine, Metoprine, Metoprolol, Metoquizine, metrifonate, Metrizamide, Metrizoate Sodium, Metronidazole, Meturedepa, Metyrapone, Metyrosine, Mexiletine Hydrochloride, Mexrenoate Potassium, Mezlocillin, mfonelic Acid, Mianserin Hydrochloride, mibefradil, Mibefradil Dihydrochloride, Mibolerone, michellamine B, Miconazole, microcolin A, Midaflur, Midazolam Hydrochloride, midodrine, mifepristone, Mifobate, miglitol, milacemide, milameline, mildronate, Milenperone, Milipertine, milnacipran, Milrinone, miltefosine, Mimbane Hydrochloride, minaprine, Minaxolone, Minocromil, Minocycline, Minoxidil, Mioflazine Hydrochloride, miokamycin, mipragoside, mirfentanil, mirimostim, Mirincamycin Hydrochloride, Mirisetron Maleate, Mirtazapine, mismatched double stranded RNA, Misonidazole, Misoprostol, Mitindomide, Mitocarcin, Mitocromin, Mitogillin, mitoguazone, mitolactol, Mitomalcin, Mitomycin, mitonafide, Mitosper, Mitotane, mitoxantrone, mivacurium chloride, mivazerol, mixanpril, Mixidine, mizolastine, mizoribine, Moclobemide, modafinil, Modaline Sulfate, Modecainide, moexipril, mofarotene, Mofegiline Hydrochloride, mofezolac, molgramostim, Molinazone, Molindone Hydrochloride, Molsidomine, mometasone, Monatepil Maleate, Monensin, Monoctanoin, Montelukast Sodium, montirelin, mopidamol, moracizine, Morantel Tartrate, Moricizine, Morniflumate, Morphine Sulfate, Morrhuate Sodium, mosapramine, mosapride, motilide, Motretinide, Moxalactam Disodium, Moxazocine, moxiraprine, Moxnidazole, moxonidine, Mumps Skin Test Antigen, mustard anticancer agent, Muzolimine, mycaperoxide B, Mycophenolic Acid, myriaporone, Nabazenil, Nabilone, Nabitan Hydrochloride, Naboctate Hydrochloride, Nabumetone, N-acetyldinaline, Nadide, nadifloxacin, Nadolol, nadroparin calcium, nafadotride, nafamostat, nafarelin, Nafcillin Sodium, Nafenopin, Nafimidone Hydrochloride, Naflocort, Nafomine Malate, Nafoxidine Hydrochloride, Nafronyl Oxalate, Naftifine Hydrochloride, naftopidil, naglivan, nagrestip, Nalbuphine Hydrochloride, Naldemedine, Nalidixate Sodium, Nalidixic Acid, nalmefene, Nalmexone Hydrochloride, naloxone+pentazocine, Naltrexone, Namoxyrate, Nandrolone Phenpropionate, Nantradol Hydrochloride, Napactadine Hydrochloride, napadisilate, Napamezole Hydrochloride, napaviin, Naphazoline Hydrochloride, naphterpin, Naproxen, Naproxol, napsagatran, Naranol Hydrochloride, Narasin, naratriptan, nartograstim, nasaruplase, Natamycin, nateplase, Naxagolide Hydrochloride, Nebivolol, Nebramycin, nedaplatin, Nedocromil, Nefazodone Hydrochloride, Neflumozide Hydrochloride, Nefopam Hydrochloride, Nelezaprine Maleate, Nemazoline Hydrochloride, nemorubicin, Neomycin Palmitate, Neostigmine Bromide, neridronic acid, Netilmicin Sulfate, neutral endopeptidase, Neutramycin, Nevirapine, Nexeridine Hydrochloride, Niacin, Nibroxane, Nicardipine Hydrochloride, Nicergoline, Niclosamide, Nicorandil, Nicotinyl Alcohol, Nifedipine, Nifirmerone, Nifluridide, Nifuradene, Nifuraldezone, Nifuratel, Nifuratrone, Nifurdazil, Nifurimide, Nifurpirinol, Nifurquinazol, Nifurthiazole, nilutamide, Nilvadipine, Nimazone, Nimodipine, niperotidine, niravoline, Niridazole, nisamycin, Nisbuterol Mesylate, nisin, Nisobamate, Nisoldipine, Nisoxetine, Nisterime Acetate, Nitarsone, nitazoxamide, nitecapone, Nitrafudam Hydrochloride, Nitralamine Hydrochloride, Nitramisole Hydrochloride, Nitrazepam, Nitrendipine, Nitrocycline, Nitrodan, Nitrofurantoin, Nitrofurazone, Nitroglycerin, Nitromersol, Nitromide, Nitromifene Citrate, Nitrous Oxide, nitroxide antioxidant, nitrullyn, Nivazol, Nivimedone Sodium, Nizatidine, Noberastine, Nocodazole, Nogalamycin, Nolinium Bromide, Nomifensine Maleate, Noracymethadol Hydrochloride, Norbolethone, Norepinephrine Bitartrate, Norethindrone, Norethynodrel, Norfloxacin, Norflurane, Norgestimate, Norgestomet, Norgestrel, Nortriptyline Hydrochloride, Noscapine, Novobiocin Sodium, N-substituted benzaimides, Nufenoxole, Nylestriol, Nystatin, O6-benzylguanine, Obidoxime Chloride, Ocaperidone, Ocfentanil Hydrochloride, Ocinaplon, Octanoic Acid, Octazamide, Octenidine Hydrochloride, Octodrine, Octreotide, Octriptyline Phosphate, Ofloxacin, Oformine, okicenone, Olanzapine, oligonucleotides, olopatadine, olprinone, olsalazine, Olsalazine Sodium, Olvanil, omeprazole, onapristone, ondansetron, Ontazolast, Oocyte maturation inhibitor, Opipramol Hydrochloride, oracin, Orconazole Nitrate, Orgotein, Orlislat, Ormaplatin, Ormetoprim, Ornidazole, Orpanoxin, Orphenadrine Citrate, osaterone, otenzepad, Oxacillin Sodium, Oxagrelate, oxaliplatin, Oxamarin Hydrochloride, oxamisole, Oxamniquine, oxandrolone, Oxantel Pamoate, Oxaprotiline Hydrochloride, Oxaprozin, Oxarbazole, Oxatomide, oxaunomycin, Oxazepam, oxcarbazepine, Oxendolone, Oxethazaine, Oxetorone Fumarate, Oxfendazole, Oxfenicine, Oxibendazole, oxiconazole, Oxidopamine, Oxidronic Acid, Oxifungin Hydrochloride, Oxilorphan, Oximonam, Oximonam Sodium, Oxiperomide, oxiracetam, Oxiramide, Oxisuran, Oxmetidine Hydrochloride, oxodipine, Oxogestone Phenpropionate, Oxolinic Acid, Oxprenolol Hydrochloride, Oxtriphylline, Oxybutynin Chloride, Oxychlorosene, Oxycodone, Oxymetazoline Hydrochloride, Oxymetholone, Oxymorphone Hydrochloride, Oxypertine, Oxyphenbutazone, Oxypurinol, Oxytetracycline, Oxytocin, ozagrel, Ozolinone, Paclitaxel, palauamine, Paldimycin, palinavir, palmitoylrhizoxin, Palmoxirate Sodium, pamaqueside, Pamatolol Sulfate, pamicogrel, Pamidronate Disodium, pamidronic acid, Panadiplon, panamesine, panaxytriol, Pancopride, Pancuronium Bromide, panipenem, pannorin, panomifene, pantethine, pantoprazole, Papaverine Hydrochloride, parabactin, Parachlorophenol, Paraldehyde, Paramethasone Acetate, Paranyline Hydrochloride, Parapenzolate Bromide, Pararosaniline Pamoate, Parbendazole, Parconazole Hydrochloride, Paregoric, Pareptide Sulfate, Pargyline Hydrochloride, parnaparin sodium, Paromomycin Sulfate, Paroxetine, parthenolide, Partricin, Paulomycin, pazelliptine, Pazinaclone, Pazoxide, pazufloxacin, pefloxacin, pegaspargase, Pegorgotein, Pelanserin Hydrochloride, peldesine, Peliomycin, Pelretin, Pelrinone Hydrochloride, Pemedolac, Pemerid Nitrate, pemirolast, Pemoline, Penamecillin, Penbutolol Sulfate, Penciclovir, Penfluridol, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentabamate, Pentaerythritol Tetranitrate, pentafuside, pentamidine, pentamorphone, Pentamustine, Pentapiperium Methylsulfate, Pentazocine, Pentetic Acid, Pentiapine Maleate, pentigetide, Pentisomicin, Pentizidone Sodium, Pentobarbital, Pentomone, Pentopril, pentosan, pentostatin, Pentoxifylline, Pentrinitrol, pentrozole, Peplomycin Sulfate, Pepstatin, perflubron, perfofamide, Perfosfamide, pergolide, Perhexiline Maleate, perillyl alcohol, Perindopril, perindoprilat, Perlapine, Permethrin, perospirone, Perphenazine, Phenacemide, phenaridine, phenazinomycin, Phenazopyridine Hydrochloride, Phenbutazone Sodium Glycerate, Phencarbamide, Phencyclidine Hydrochloride, Phendimetrazine Tartrate, Phenelzine Sulfate, Phenmetrazine Hydrochloride, Phenobarbital, Phenoxybenzamine Hydrochloride, Phenprocoumon, phenserine, phensuccinal, Phensuximide, Phentermine, Phentermine Hydrochloride, phentolamine mesilate, Phentoxifylline, Phenyl Aminosalicylate, phenylacetate, Phenylalanine, phenylalanyl ketoconazole, Phenylbutazone, Phenylephrine Hydrochloride, Phenylpropanolamine Hydrochloride, Phenylpropanolamine Polistirex, Phenyramidol Hydrochloride, Phenyloin, phosphatase inhibitors, Physostigmine, picenadol, picibanil, Picotrin Diolamine, picroliv, picumeterol, pidotimod, Pifamine, Pilocarpine, pilsicainide, pimagedine, Pimetine Hydrochloride, pimilprost, Pimobendan, Pimozide, Pinacidil, Pinadoline, Pindolol, pinnenol, pinocebrin, Pinoxepin Hydrochloride, pioglitazone, Pipamperone, Pipazethate, pipecuronium bromide, Piperacetazine, Piperacillin Sodium, Piperamide Maleate, piperazine, Pipobroman, Piposulfan, Pipotiazine Palmitate, Pipoxolan Hydrochloride, Piprozolin, Piquindone Hydrochloride, Piquizil Hydrochloride, Piracetam, Pirandamine Hydrochloride, pirarubicin, Pirazmonam Sodium, Pirazolac, Pirbenicillin Sodium, Pirbuterol Acetate, Pirenperone, Pirenzepine Hydrochloride, piretamide, Pirfenidone, Piridicillin Sodium, Piridronate Sodium, Piriprost, piritrexim, Pirlimycin Hydrochloride, pirlindole, pirmagrel, Pirmenol Hydrochloride, Pirnabine, Piroctone, Pirodavir, pirodomast, Pirogliride Tartrate, Pirolate, Pirolazamide, Piroxantrone Hydrochloride, Piroxicam, Piroximone, Pirprofen, Pirquinozol, Pirsidomine, Prenylamine, Pituitary, Posterior, Pivampicillin Hydrochloride, Pivopril, Pizotyline, placetin A, platinum compounds, platinum-triamine complex, Plicamycin, Plomestane, Pobilukast Edamine, Podofilox, Poisonoak Extract, Poldine Methylsulfate, Poliglusam, Polignate Sodium, Polymyxin B Sulfate, Polythiazide, Ponalrestat, Porfimer Sodium, Porfiromycin, Potassium Chloride, Potassium Iodide, Potassium Permanganate, Povidone-Iodine, Practolol, Pralidoxime Chloride, Pramiracetam Hydrochloride, Pramoxine Hydrochloride, Pranolium Chloride, Pravadoline Maleate, Pravastatin (Pravachol), Prazepam, Prazosin, Prazosin Hydrochloride, Prednazate, Prednicarbate, Prednimustine, Prednisolone, Prednisone, Prednival, Pregnenolone Succiniate, Prenalterol Hydrochloride, Pridefine Hydrochloride, Prifelone, Prilocalne Hydrochloride, Prilosec, Primaquine Phosphate, Primidolol, Primidone, Prinivil, Prinomide Tromethamine, Prinoxodan, Prizidilol Hydrochloride, Proadifen Hydrochloride, Probenecid, Probicromil Calcium, Probucol, Procainamide Hydrochloride, Procaine Hydrochloride, Procarbazine Hydrochloride, Procaterol Hydrochloride, Prochlorperazine, Procinonide, Proclonol, Procyclidine Hydrochloride, Prodilidine Hydrochloride, Prodolic Acid, Profadol Hydrochloride, Progabide, Progesterone, Proglumide, Proinsulin Human, Proline, Prolintane Hydrochloride, Promazine Hydrochloride, Promethazine Hydrochloride, Propafenone Hydrochloride, propagermanium, Propanidid, Propantheline Bromide, Proparacaine Hydrochloride, Propatyl Nitrate, propentofylline, Propenzolate Hydrochloride, Propikacin, Propiomazine, Propionic Acid, propionylcarnitine, L-, propiram, propiram+paracetamol, propiverine, Propofol, Propoxycaine Hydrochloride, Propoxyphene Hydrochloride, Propranolol Hydrochloride, Propulsid, propyl bisacridone, Propylhexedrine, Propyliodone, Propylthiouracil, Proquazone, Prorenoate Potassium, Proroxan Hydrochloride, Proscillaridin, Prostalene, prostratin, Protamine Sulfate, protegrin, Protirelin, protosufloxacin, Protriptyline Hydrochloride, Proxazole, Proxazole Citrate, Proxicromil, Proxorphan Tartrate, prulifloxacin, Pseudoephedrine Hydrochloride, Puromycin, purpurins, Pyrabrom, Pyrantel Pamoate, Pyrazinamide, Pyrazofurin, pyrazoloacridine, Pyridostigmine Bromide, Pyrilamine Maleate, Pyrimethamine, Pyrinoline, Pyrithione Sodium, Pyrithione Zinc, Pyrovalerone Hydrochloride, Pyroxamine Maleate, Pyrrocaine, Pyrroliphene Hydrochloride, Pyrrolnitrin, Pyrvinium Pamoate, Quadazocine Mesylate, Quazepam, Quazinone, Quazodine, Quazolast, quetiapine, quiflapon, quinagolide, Quinaldine Blue, quinapril, Quinaprilat, Quinazosin Hydrochloride, Quinbolone, Quinctolate, Quindecamine Acetate, Quindonium Bromide, Quinelorane Hydrochloride, Quinestrol, Quinfamide, Quingestanol Acetate, Quingestrone, Quinidine Gluconate, Quinielorane Hydrochloride, Quinine Sulfate, Quinpirole Hydrochloride, Quinterenol Sulfate, Quinuclium Bromide, Quinupristin, Quipazine Maleate, Rabeprazole Sodium, Racephenicol, Racepinephrine, raf antagonists, Rafoxamide, Ralitoline, raloxifene, raltitrexed, ramatroban, Ramipril, Ramoplanin, ramosetron, ranelic acid, Ranimycin, Ranitidine, ranolazine, Rauwolfia Serpentina, recainam, Recainam Hydrochloride, Reclazepam, regavirumab, Regramostim, Relaxin, Relomycin, Remacemide Hydrochloride, Remifentanil Hydrochloride, Remiprostol, Remoxipride, Repirinast, Repromicin, Reproterol Hydrochloride, Reserpine, resinferatoxin, Resorcinol, retelliptine demethylated, reticulon, reviparin sodium, revizinone, rhenium Re 186 etidronate, rhizoxin, Ribaminol, Ribavirin, Riboprine, ribozymes, ricasetron, Ridogrel, Rifabutin, Rifametane, Rifamexil, Rifamide, Rifampin, Rifapentine, Rifaximin, RH retinamide, rilopirox, Riluzole, rimantadine, Rimcazole Hydrochloride, Rimexolone, Rimiterol Hydrobromide, rimoprogin, riodipine, Rioprostil, Ripazepam, ripisartan, Risedronate Sodium, risedronic acid, Risocaine, Risotilide Hydrochloride, rispenzepine, Risperdal, Risperidone, Ritanserin, ritipenem, Ritodrine, Ritolukast, ritonavir, rizatriptan benzoate, Rocastine Hydrochloride, Rocuronium Bromide, Rodocaine, Roflurane, Rogletimide, rohitukine, rokitamycin, Roletamicide, Rolgamidine, Rolicyprine, Rolipram, Rolitetracycline, Rolodine, Romazarit, romurtide, Ronidazole, ropinirole, Ropitoin Hydrochloride, ropivacaine, Ropizine, roquinimex, Rosaramicin, rosiglitazone, Rosoxacin, Rotoxamine, roxaitidine, Roxarsone, roxindole, roxithromycin, rubiginone B1, ruboxyl, rufloxacin, rupatidine, Rutamycin, ruzadolane, Sabeluzole, safingol, safironil, saintopin, salbutamol, R-Salcolex, Salethamide Maleate, Salicyl Alcohol, Salicylamide, Salicylate Meglumine, Salicylic Acid, Salmeterol, Salnacediin, Salsalate, sameridine, sampatrilat, Sancycline, sanfetrinem, Sanguinarium Chloride, Saperconazole, saprisartan, sapropterin, saquinavir, Sarafloxacin Hydrochloride, Saralasin Acetate, SarCNU, sarcophytol A, sargramostim, Sarmoxicillin, Sarpicillin, sarpogrelate, saruplase, saterinone, satigrel, satumomab pendetide, Schick Test Control, Scopafungin, Scopolamine Hydrobromide, Scrazaipine Hydrochloride, Sdi 1 mimetics, Secalciferol, Secobarbital, Seelzone, Seglitide Acetate, selegiline, Selegiline Hydrochloride, Selenium Sulfide, Selenomethionine Se 75, Selfotel, sematilide, semduramicin, semotiadil, semustine, sense oligonucleotides, Sepazonium Chloride, Seperidol Hydrochloride, Seprilose, Seproxetine Hydrochloride, Seractide Acetate, Sergolexole Maleate, Serine, Sermetacin, Sermorelin Acetate, sertaconazole, sertindole, sertraline, setiptiline, Setoperone, sevirumab, sevoflurane, sezolamide, Sibopirdine, Sibutramine Hydrochloride, signal transduction inhibitors, Silandrone, silipide, silteplase, Silver Nitrate, simendan, Simtrazene, Simvastatin, Sincalide, Sinefungin, sinitrodil, sinnabidol, sipatrigine, sirolimus, Sisomicin, Sitogluside, sizofiran, sobuzoxane, Sodium Amylosulfate, Sodium Iodide I 123, Sodium Nitroprusside, Sodium Oxybate, sodium phenylacetate, Sodium Salicylate, solverol, Solypertine Tartrate, Somalapor, Somantadine Hydrochloride, somatomedin B, somatomedin C, somatrem, somatropin, Somenopor, Somidobove, sonermin, Sorbinil, Sorivudine, sotalol, Soterenol Hydrochloride, Sparfloxacin, Sparfosate Sodium, sparfosic acid, Sparsomycin, Sparteine Sulfate, Spectinomycin Hydrochloride, spicamycin D, Spiperone, Spiradoline Mesylate, Spiramycin, Spirapril Hydrochloride, Spiraprilat, Spirogermanium Hydrochloride, Spiromustine, Spironolactone, Spiroplatin, Spiroxasone, splenopentin, spongistatin 1, Sprodiamide, squalamine, Stallimycin Hydrochloride, Stannous Pyrophosphate, Stannous Sulfur Colloid, Stanozolol, Statolon, staurosporine, stavudine, Steffimycin, Stenbolone Acetate, stepronin, Stilbazium Iodide, Stilonium Iodide, stipiamide, Stiripentol, stobadine, Streptomycin Sulfate, Streptonicozid, Streptonigrin, Streptozocin, stromelysin inhibitors, Strontium Chloride Sr 89, succibun, Succimer, Succinylcholine Chloride, Sucralfate, Sucrosofate Potassium, Sudoxicam, Sufentanil, Sufotidine, Sulazepam, Sulbactam Pivoxil, Sulconazole Nitrate, Sulfabenz, Sulfabenzamide, Sulfacetamide, Sulfacytine, Sulfadiazine, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, sulfasalazine, Sulfasomizole, Sulfazamet, Sulfinalol Hydrochloride, sulfinosine, Sulfinpyrazone, Sulfisoxazole, Sulfomyxin, Sulfonterol Hydrochloride, sulfoxamine, Sulinldac, Sulmarin, Sulnidazole, Suloctidil, Sulofenur, sulopenem, Suloxifen Oxalate, Sulpiride, Sulprostone, sultamicillin, Sulthiame, sultopride, sulukast, Sumarotene, sumatriptan, Suncillin Sodium, Suproclone, Suprofen, suradista, suramin, Surfomer, Suricainide Maleate, Suritozole, Suronacrine Maleate, Suxemerid Sulfate, swainsonine, symakalim, Symclosene, Symetine Hydrochloride, synthetic glycosaminoglycans, Taciamine Hydrochloride, Tacrine Hydrochloride, Tacrolimus, Talampicillin Hydrochloride, Taleranol, Talisomycin, tallimustine, Talmetacin, Talniflumate, Talopram Hydrochloride, Talosalate, Tametraline Hydrochloride, Tamoxifen, Tampramine Fumarate, Tamsulosin Hydrochloride, Tandamine Hydrochloride, tandospirone, tapgen, taprostene, Tasosartan, tauromustine, Taxane, Taxoid, Tazadolene Succinate, tazanolast, tazarotene, Tazifylline Hydrochloride, Tazobactam, Tazofelone, Tazolol Hydrochloride, Tebufelone, Tebuquine, Technetium Tc 99 m Bicisate, Teclozan, Tecogalan Sodium, Teecleukin, Teflurane, Tegafur, Tegretol, Teicoplanin, telenzepine, tellurapyrylium, telmesteine, telmisartan, telomerase inhibitors, Teloxantrone Hydrochloride, Teludipine Hydrochloride, Temafloxacin Hydrochloride, Tematropium Methyl sulfate, Temazepam, Temelastine, temocapril, Temocillin, temoporfin, temozolomide, Tenidap, Teniposide, tenosal, tenoxicam, tepirindole, Tepoxalin, Teprotide, terazosin, Terbinafine, Terbutaline Sulfate, Terconazole, terfenadine, terflavoxate, terguride, Teriparatide Acetate, terlakiren, terlipressin, terodiline, Teroxalene Hydrochloride, Teroxirone, tertatolol, Tesicam, Tesimide, Testolactone, Testosterone, Tetracaine, tetrachlorodecaoxide, Tetracycline, Tetrahydrozoline Hydrochloride, Tetramisole Hydrochloride, Tetrazolast Meglumine, tetrazomine, Tetrofosmin, Tetroquinone, Tetroxoprim, Tetrydamine, thaliblastine, Thalidomide, Theofibrate, Theophylline, Thiabendazole, Thiamiprine, Thiamphenicol, Thiamylal, Thiazesim Hydrochloride, Thiazinamium Chloride, Thiethylperazine, Thimerfonate Sodium, Thimerosal, thiocoraline, thiofedrine, Thioguanine, thiomarinol, Thiopental Sodium, thioperamide, Thioridazine, Thiotepa, Thiothixene, Thiphenamil Hydrochloride, Thiphencillin Potassium, Thiram, Thozalinone, Threonine, Thrombin, thrombopoietin, thrombopoietin mimetic, thymalfasin, thymopoietin receptor agonist, thymotrinan, Thyromedan Hydrochloride, Thyroxine 1 125, Thyroxine 1 131, Tiacrilast, Tiacrilast Sodium, tiagabine, Tiamenidine, tianeptine, tiapafant, Tiapamil Hydrochloride, Tiaramide Hydrochloride, Tiazofurin, Tibenelast Sodium, Tibolone, Tibric Acid, Ticabesone Propionate, Ticarbodine, Ticarcillin Cresyl Sodium, Ticlatone, ticlopidine, Ticrynafen, tienoxolol, Tifurac Sodium, Tigemonam Dicholine, Tigestol, Tiletamine Hydrochloride, Tilidine Hydrochloride, tilisolol, tilnoprofen arbamel, Tilorone Hydrochloride, Tiludronate Disodium, tiludronic acid, Timefurone, Timobesone Acetate, Timolol, tin ethyl etiopurpurin, Tinabinol, Timidazole, Tinzaparin Sodium, Tioconazole, Tiodazosin, Tiodonium Chloride, Tioperidone Hydrochloride, Tiopinac, Tiospirone Hydrochloride, Tiotidine, tiotropium bromide, Tioxidazole, Tipentosin Hydrochloride, Tipredane, Tiprenolol Hydrochloride, Tiprinast Meglumine, Tipropidil Hydrochloride, Tiqueside, Tiquinamide Hydrochloride, tirandalydigin, Tirapazamine, tirilazad, tirofiban, tiropramide, titanocene dichloride, Tixanox, Tixocortol Pivalate, Tizanidine Hydrochloride, Tobramycin, Tocainide, Tocamphyl, Tofenacin Hydrochloride, Tolamolol, Tolazamide, Tolazoline Hydrochloride, Tolbutamide, Tolcapone, Tolciclate, Tolfamide, Tolgabide, lamotrigine, Tolimidone, Tolindate, Tolmetin, Tolnaftate, Tolpovidone 1 131, Tolpyrramide, Tolrestat, Tomelukast, Tomoxetine Hydrochloride, Tonazocine Mesylate, Topiramate, topotecan, Topotecan Hydrochloride, topsentin, Topterone, Toquizine, torasemide, toremifene, Torsemide, Tosifen, Tosufloxacin, totipotent stem cell factor, Tracazolate, trafermin, Tralonide, Tramadol Hydrochloride, Tramazoline Hydrochloride, trandolapril, Tranexamic Acid, Tranilast, Transcainide, translation inhibitors, traxanox, Trazodone Hydrochloride, Trazodone-HCL, Trebenzomine Hydrochloride, Trefentanil Hydrochloride, Treloxinate, Trepipam Maleate, Trestolone Acetate, tretinoin, Triacetin, triacetyluridine, Triafungin, Triamcinolone, Triampyzine Sulfate, Triamterene, Triazolam, Tribenoside, tricaprilin, Tricetamide, Trichlormethiazide, trichohyalin, triciribine, Tricitrates, Triclofenol piperazine, Triclofos Sodium, Triclonide, trientine, Trifenagrel, triflavin, Triflocin, Triflubazam, Triflumidate, Trifluoperazine Hydrochloride, Trifluperidol, Triflupromazine, Triflupromazine Hydrochloride, Trifluridine, Trihexyphenidyl Hydrochloride, Trilostane, Trimazosin Hydrochloride, trimegestone, Trimeprazine Tartrate, Trimethadione, Trimethaphan Camsylate, Trimethobenzamide Hydrochloride, Trimethoprim, Trimetozine, Trimetrexate, Trimipramine, Trimoprostil, Trimoxamine Hydrochloride, Triolein 1 125, Triolein 1 131, Trioxifene Mesylate, Tripamide, Tripelennamine Hydrochloride, Triprolidine Hydrochloride, Triptorelin, Tri sulfapyrimidines, Troclosene Potassium, troglitazone, Trolamine, Troleandomycin, trombodipine, trometamol, Tropanserin Hydrochloride, Tropicamide, tropine ester, tropisetron, trospectomycin, trovafloxacin, trovirdine, Tryptophan, Tuberculin, Tubocurarine Chloride, Tubulozole Hydrochloride, tucarcsol, tulobuterol, turosteride, Tybamate, tylogenin, Tyropanoate Sodium, Tyrosine, Tyrothricin, tyrphostins, ubenimex, Uldazepam, Undecylenic Acid, Uracil Mustard, urapidil, Urea, Uredepa, uridine triphosphate, Urofollitropin, Urokinase, Ursodiol, valaciclovir, Valine, Valnoctamide, Valproate Sodium, Valproic Acid, valsartan, vamicamide, vanadeine, Vancomycin, vaninolol, Vapiprost Hydrochloride, Vapreotide, variolin B, Vasopressin, Vecuronium Bromide, velaresol, Velnacrine Maleate, venlafaxine, Veradoline Hydrochloride, veramine, Verapamil Hydrochloride, verdins, Verilopam Hydrochloride, Verlukast, Verofylline, veroxan, verteporfin, Vesnarinone, vexibinol, Vidarabine, vigabatrin, Viloxazine Hydrochloride, Vinblastine Sulfate, vinburnine citrate, Vincofos, vinconate, Vincristine Sulfate, Vindesine, Vindesine Sulfate, Vinepidine Sulfate, Vinglycinate Sulfate, Vinleurosine Sulfate, vinorelbine, vinpocetine, vintoperol, vinxaltine, Vinzolidine Sulfate, Viprostol, Virginiamycin, Viridofulvin, Viroxime, vitaxin, Volazocine, voriconazole, vorozole, voxergolide, Warfarin Sodium, Xamoterol, Xanomeline, Xanoxate Sodium, Xanthinol Niacinate, xemilofiban, Xenalipin, Xenbucin, Xilobam, ximoprofen, Xipamide, Xorphanol Mesylate, Xylamidine Tosylate, Xylazine Hydrochloride, Xylometazoline Hydrochloride, Xylose, yangambin, zabicipril, zacopride, zafirlukast, Zalcitabine, zaleplon, zalospirone, Zaltidine Hydrochloride, zaltoprofen, zanamivir, zankiren, zanoterone, Zantac, Zarirlukast, zatebradine, zatosetron, Zatosetron Maleate, zenarestat, Zenazocine Mesylate, Zeniplatin, Zeranol, Zidometacin, Zidovudine, zifrosilone, Zilantel, zilascorb, zileuton, Zimeldine Hydrochloride, Zinc Undecylenate, Zindotrine, Zinoconazole Hydrochloride, Zinostatin, Zinterol Hydrochloride, Zinviroxime, ziprasidone, Zobolt, Zofenopril Calcium, Zofenoprilat, Zolamine Hydrochloride, Zolazepam Hydrochloride, zoledronie acid, Zolertine Hydrochloride, zolmitriptan, zolpidem, Zomepirac Sodium, Zometapine, Zoniclezole Hydrochloride, Zonisamide, zopiclone, Zopolrestat, Zorbamyciin, Zorubicin Hydrochloride, zotepine, Zucapsaicin. Another pharmaceutical active acceptable for use herein is lumateperone, as disclosed in

U.S. Pat. Nos. 9,745,300, 9,708,322, 7,183,282, 7,071,186, 6,552,017, 8,648,077, 8,598,119, 9,751,883, 9,371,324, 9,315,504, 9,428,506, 8,993,572, 8,309,722, 6,713,471, 8,779,139, 9,168,258, RE039680E1, 9,616,061, 9,586,960, and in U.S. Patent Publication Nos. 2017/0114037, 2017/0183350, 2015/0072964, 2004/0034015, 2017/0189398, 2016/0310502, 2015/0080404, the aforementioned contents of which are incorporated by reference herein in their entirety.

Further examples of antidiabetic actives include but not limited to JTT-501 (PNU-182716) (Reglitazar), AR-H039242, MCC-555 (Netoglitazone), AR-H049020 Tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (Ragaglitazar), L-686398, L-168049, L-805645, L-054852, Demethyl asteriquinone B1 (L-783281), L-363586, KRP-297, P32/98, CRE-16336 and EML-16257.

Erectile dysfunction therapies useful herein include, but are not limited to, agents for facilitating blood flow to the penis, and for effecting autonomic nervous activities, such as increasing parasympathetic (cholinergic) and decreasing sympathetic (adrenergic) activities. Useful actives for treatment of erectile dysfunction include, for example, but are not limited to, alprostadil, tadalafil, vardenafil, apomorphine, yohimbine hydrochloride, sildenafil citrate, sildenafil and any combination thereof. In an embodiment, the active is tadalafil.

Actives or medications for the treatment of headaches and/or migraines may also be used herein. Examples of specific actives include, but are not limited to, triptans, such as eletriptan, naratriptan, rizatriptan (rizatriptan benzoate), sumatriptan, and zolmitriptan. In an embodiment, the active is rizatriptan, optionally in combination with an NSAID.

In certain embodiments, the pharmaceutically active component can be epinephrine, a prodrug, analog, derivative or salt of epinephrine.

In one example, a composition including a prodrug, such as a prodrug for epinephrine, can have a biodelivery profile similar to that of epinephrine administered by injection.

In certain examples, the composition can include a combination of epinephrine and a prodrug. In other examples, the composition can include a combination of two or more prodrugs. In other examples, the composition can include epinephrine and a combination of two or more prodrugs.

Epinephrine or its prodrug can be present in an amount of from about 0.01 mg to about 100 mg per dosage, for example, at a 0.1 mg, 5 mg, 10 mg, 20 mg, 30 mg, 40 mg, 50 mg, 60 mg, 70 mg, 80 mg, 90 mg or 100 mg dosage, including greater than 0.1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. The epinephrine or prodrug can be provided in a single dose. The epinephrine or prodrug can also be provided in two or more doses.

Dipivefrin can be present in an amount of from about 0.5 mg to about 100 mg per dosage, for example, at a 0.5mg, 1 mg, 5 mg, 10 mg, 20 mg, 30 mg, 40 mg, 50 mg, 60 mg, 70 mg, 80 mg, 90 mg or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. Dipivefrin can be provided in a single dose. Dipivefrin can also be provided in two or more doses.

### Prodrug Composition

Administering epinephrine as a prodrug such as dipivefrin, or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15 confer certain advantages. For one, dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 are lipophilic and therefore has a higher permeation through lung tissue. Dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14,AQEP-15, AQEP-16, AQEP-17, AQEP-18, AQEP-19, AQEP-20, AQEP-21, AQEP-22, AQEP-23, AQEP-24, AQEP-25, AQEP-26, AQEP-27 and AQEP-28 each have a longer plasma half-life due to higher protein binding. Dipivefrin is capable of sustained blood levels, and has a reduced interaction with a-receptors, therefore minimizing or eliminating unwanted or harmful vasoconstriction. Prodrugs, for example, AQEP-09, can exhibit higher binding affinity for α- and β-receptors, with binding and activation profiles that are more similar to epinephrine than dipivefrin. Other prodrugs, and combinations of prodrugs, can exhibit binding affinities for α- and β-receptors that favor one or more receptor, similar to or different from epinephrine.

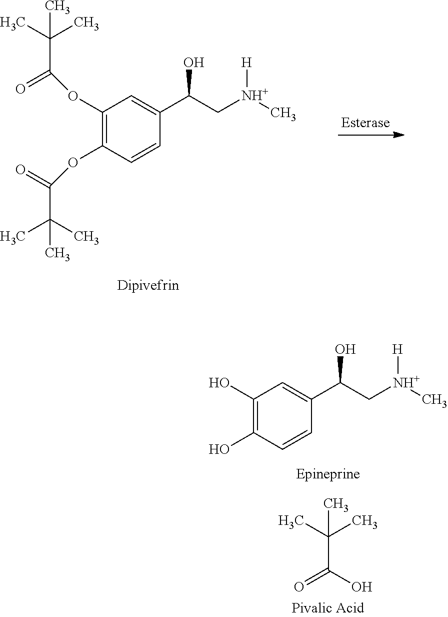

Dipivefrin or prodrugs AQEP-03, AQEP-04, AQEP-05, AQEP-06, AQEP-07, AQEP-08, AQEP-09, AQEP-10, AQEP-11, AQEP-12, AQEP-13, AQEP-14, AQEP-15, AQEP-16, AQEP-17, AQEP-18, AQEP-19, AQEP-20, AQEP-21, AQEP-22, AQEP-23, AQEP-24, AQEP-25, AQEP-26, AQEP-27 and AQEP-28 alone or in combination, can be delivered by inhalation in a similar manner as with epinephrine. See, for example, Breuer et al. Eur J Clin Pharmacol (2013) 69:1303-1310, or Kerwin et al. Journal Of Aerosol Medicine And Pulmonary Drug Delivery Volume 33, Number 5, 2020, each of which is incorporated by reference in its entirety.

Steric hindrance is the slowing of chemical reactions due to steric bulk. It is usually manifested in intermolecular reactions such as enzymatic reactions. Steric hindrance is often exploited to control selectivity, such as slowing unwanted side-reactions. In pharmacology, steric effects determine how and at what rate a drug will interact with its target bio-molecules. The design of a prodrug needs to account for steric hindrance resulting from the prodrug substituents and its interactions with respective enzymes, including hydrolases, esterases and amidases for example. Additives, such as those described below, can also impact the activity and/or interaction with enzymes. In certain embodiments, one or more of these enzymes can be endogenous. In other embodiments, one or more of these enzymes can be exogenous. Stereospecific nucleophilic attack on substituted carbon atoms is a simple and versatile way to construct stereocenter next to heteroatoms with overall inversion of stereochemistry. A tertiary group adjacent to the ester unexpectedly impedes hydrolysis more when compared to non-tertiary groups.

Additives may be included in the composition. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, salts, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutically active component(s). Excipients refer to compounds or particles that optimize the formulation, for example, by increasing its flowability. As used herein, the term “stabilizer” means an excipient capable of preventing aggregation or other physical degradation, as well as chemical degradation, of the active pharmaceutical ingredient, another excipient, or the combination thereof.

Stabilizers may also be classified as antioxidants, sequestrants, pH modifiers, emulsifiers and/or surfactants, or UV stabilizers.

Antioxidants (i.e., pharmaceutically compatible compound(s) or composition(s) that decelerates, inhibits, interrupts and/or stops oxidation processes) include, in particular, the following substances: tocopherols and the esters thereof, sesamol of sesame oil, coniferyl benzoate of benzoin resin, nordihydroguaietic resin and nordihydroguaiaretic acid (NDGA), gallic acid, gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, ethylenediamine tetra acetic acid (EDTA), ethylene glycol-bis(β-aminoethyl ether)-N,N,N′,N′-tetraacetic acid (EGTA), butylated hydroxytoluene (BHT) (including t-butylhydroxytoluene), cysteine, ferulic acid, caffeic acid, tannic acid, uric acid, and propionic acid. Typical antioxidants are tocopherol such as, for example, α-tocopherol and the esters thereof, butylated hydroxytoluene and butylated hydroxyanisole. The terms “tocopherol” also includes esters of tocopherol. A known tocopherol is α-tocopherol. The term “α-tocopherol” includes esters of α-tocopherol (for example, α-tocopherol acetate).

Sequestrants (i.e., any compounds which can engage in host-guest complex formation with another compound, such as the active ingredient or another excipient; also referred to as a sequestering agent) include calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more α-D-mannopyranose units linked at the 1,4 positions by α linkages), cyclogalactins (5 or more β-D-galactopyranose units linked at the 1,4 positions by β linkages), cycloaltrins (5 or more α-D-altropyranose units linked at the 1,4 positions by α linkages), and combinations thereof.

pH modifiers or stabilizers include acids (e.g., hydrochloric acid, hydrofluoric acid, tartaric acid, citric acid, lactic acid, fumaric acid, phosphoric acid, ascorbic acid, acetic acid, succinic acid, propanoic acid, butyric acid, isobutyric acid, pivalic acid, malic acid, tartaric acid, adipic acid and maleic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

Examples of emulsifiers and/or surfactants include poloxamers or pluronics, polyethylene glycols, polyethylene glycol monostearate, polysorbates, sodium lauryl sulfate, polyethoxylated and hydrogenated castor oil, alkyl polyoside, a grafted water soluble protein on a hydrophobic backbone, lecithin, glyceryl monostearate, glyceryl monooleate, glyceryl monostearate/polyoxyethylene stearate, ketostearyl alcohol/sodium lauryl sulfate, carbomer, phospholipids, (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C₁₀-C₂₄)-alkyl-dimethylammonium chloride or bromide), (C₁₀-C₂₄)-alkyl-dimethylethylammonium chloride or bromide, (C₁₀-C₂₄)-alkyl-trimethylammonium chloride or bromide (e.g., cetyltrimethylammonium chloride or bromide), (C₁₀-C₂₄)-alkyl-dimethylbenzylammonium chloride or bromide (e.g., (C₁₂-C₁₈)-alkyl-dimethylbenzylammonium chloride), N—(C₁₀-C₁₈)-alkyl-pyridinium chloride or bromide (e.g., N—(C₁₂-C₁₆)-alkyl-pyridinium chloride or bromide), N—(C₁₀-C₁₈)-alkyl-isoquinolinium chloride, bromide or monoalkyl sulfate, N—(C₁₂-C₁₈)-alkyl-polyoylaminoformylmethylpyridinium chloride, N—(C₁₂-C18)-alkyl-N-methylmorpholinium chloride, bromide or monoalkyl sulfate, N—(C₁₂-C₁₈)-alkyl-N-ethylmorpholinium chloride, bromide or monoalkyl sulfate, (C₁₆-C₁₈)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate (in the foregoing, “acyl” standing for, e.g., stearyl or oleyl), and combinations thereof.

The composition can include a pulmonary surfactant, for example, a material similar to a lipo-protein substance naturally produced in the lungs that are essential for proper breathing, alveolar stability and gas exchange. Pulmonary surfactants can be surface-active agents naturally formed by type II alveolar cells that reduce the surface tension at the air-liquid interface of alveoli. Pulmonary surfactants are generally made up of about 90% lipids (about half of which is the phospolipid dipalmitoylphosphatidylcholine (DPPC)) and about 10% protein. At least four native surfactants have been identified: SP-A, B, C, and D. The hydrophobic surfactant proteins B (SP-B) and C (SP-C) are tightly bound to the phospholipids, and promote their adsorption into the air-liquid interface of the alveoli. These proteins are critical for formation of the surfactant composition. The term “surfactant” also includes currently available surfactant preparations, including, but not limited to, Survanta® (beractant), Infasurf® (calfactant), Exosurf neonatal® (colfosceril palmitate), Curosurf® (poractant alfa), Surfaxing (lucinactant), Aerosurf® (aerosolized Surfaxing), Vanticute® (lusupultide), Alveofact® (bovactant), among others.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers include ascorbyl palmitate, ascorbic acid, alpha tocopherol, butylated hydroxytoluene, butylated hydroxyanisole, cysteine HCl, citric acid, ethylenediamine tetra acetic acid (EDTA), methionine, sodium citrate, sodium ascorbate, sodium thiosulfate, sodium metabisulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-camosine, histamine, imidazole 4-acetic acid), indoles (e.g., tryptophan and derivatives thereof, such as N-acetyl-5-methoxytryptamine, N-acetyl serotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, o-phenanthroline, and citrate. “Detergents” include, but are not limited to, SDS and sodium lauroyl sarcosyl. “Chaotropes” include, but are not limited to guandinium hydrochloride, isothiocyanate, urea, and formamide. As discussed herein, stabilizers can be present in 0.0001%-50% by weight, including greater than 0.0001%, greater than 0.001%, greater than 0.01%, greater than 0.1%, greater than 1%, greater than 5%, greater than 10%, greater than 20%, greater than 30%, greater than 40%, greater than 50%, less than 50%, less than 40%, less than 30%, less than 20%, less than 10%, less than 1%, less than 0.1%, less than 0.01%, less than 0.001%, or less than 0.0001% by weight.

Useful additives can include, for example, gelatin, gelatin hydrosylates, recombinant gelatin, vegetable proteins such as sunflower protein, soybean proteins, cotton seed proteins, peanut proteins, grape seed proteins, whey proteins, whey protein isolates, blood proteins, egg proteins, acrylated proteins, polysaccharides or carbohydrates such as gum arabica, chitin, chitosan, xanthan gum, agar, gum ghatti, chondroitin sulfate, dextran, carrageenans, gum karaya, hyaluronic acid, curdian, alginic acid, gum tragacanth, pullulan, laminarin, khaya, zanflo, albizia gums, guar gum, Baker's yeast, locust bean gum, glycan, starch, schizophyllan, amylase, lentinan, cellulose, krestin, pectin, scleroglucan, larch gum, potato starch, pea starch, hetastarch, starch acetate, starch phosphates, inulin, and pectin, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVA/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; or other similar polymers.

Stabilizers can include nanoparticulate stabilizers, such as a dispersant layer around a nanoparticulate surface. See, e.g., Langmuir 2007, (23)3, 1081-1090, Dec. 20, 2006, doi.org/10.1021/1a062042s, which is incorporated by reference in its entirety. Stabilizers can include stabilizer ligands, e.g., monomers bearing functional groups that can be chemisorbed on nanoparticles to form polymerizable monolayers. See, e.g., Jadhav et al doi.org/10.1002/ppsc.201400074, which is incorporated by reference in its entirety. Stabilizers can include surface stabilizers. See, e.g., U.S. Pat. No. 6,428,814 and Japanese Pat. JP 4,598,399B2, each of which is incorporated by reference in its entirety. Surface stabilizers can include tyloxapol (U.S. Pat. No. 5,429,824), polyalkylene block copolymers (U.S. Pat. No. 5,565,188), sulfated non-ionic block copolymers (U.S. Pat. No. 5,569,448), high molecular weight, linear, poly(ethylene oxide) polymers (U.S. Pat. No. 5,580,579), butylene oxide-ethylene oxide block copolymers (U.S. Pat. No. 5,587,143), hydroxypropyl cellulose (U.S. Pat. No. 5,591,456), and sugar based surface stabilizers (U.S. Pat. No. 5,622,938), each of which is incorporated by reference in its entirety. Stabilizers can include peptide stabilizers. See, e.g., WO2006097748A2, which is incorporated by reference in its entirety. Stabilizers can include for example, L-cysteine hydrochloride, glycine hydrochloride, malic acid, sodium metabisulfite, citric acid, tartaric acid, and L-cystine dihydrochloride. See, e.g., U.S. Pat. 6,153,223, which is incorporated by reference in its entirety. Stabilizers can include natural compounds. Stabilizers can include synthetic compounds. Stabilizers can include a blend of one of more compounds or categories of compounds described above. Stabilizers can be function to protect the metabolism of a prodrug until a desired time or until it reaches a specific target, tissue or environment.

The additional components can range up to about 80%, desirably about 0.005% to 50% and more desirably within the range of 1% to 20% based on the weight of all composition components, including greater than 1%, greater than 5%, greater than 10%, greater than 20%, greater than 30%, greater than 40%, greater than 50%, greater than 60%, greater than 70%, about 80%, greater than 80%, less than 80%, less than 70%, less than 60%, less than 50%, less than 40%, less than 30%, less than 20%, less than 10%, less than 5%, about 3%, or less than 1%.

Other additives can include anti-tacking, flow agents and opacifiers, such as the oxides of magnesium aluminum, silicon, titanium, etc. desirably in a concentration range of about 0.005% to about 5% by weight and desirably about 0.02% to about 2% based on the weight of all composition components, including greater than 0.02%, greater than 0.2%, greater than 0.5%, greater than 1%, greater than 1.5%, greater than 2%, greater than 4%, about 5%, greater than 5%, less than 4%, less than 2%, less than 1%, less than 0.5%, less than 0.2%, or less than 0.02%.

Other suitable additives to the composition can include water; terpenes, such as menthol; alcohols, such as ethanol, propylene glycol, glycerol and other similar alcohols; dimethylformamide; dimethylacetamide; wax; and mixtures thereof.

In certain embodiments, the composition can include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols sorbitol, sodium diethylsulfosuccinate, triethyl citrate, tributyl citrate, phytoextracts, fatty acid esters, fatty acids, oils and the like, added in concentrations ranging from about 0.1% to about 40%, and desirably ranging from about 0.5% to about 20% based on the weight of the composition including greater than 0.5%, greater than 1%, greater than 1.5%, greater than 2%, greater than 4%, greater than 5%, greater than 10%, greater than 15%, about 20%, greater than 20%, less than 20%, less than 15%, less than 10%, less than 5%, less than 4%, less than 2%, less than 1%, or less than 0.5%. In certain embodiments, there may further be added compounds to improve the texture properties of the composition material such as animal or vegetable fats, desirably in their hydrogenated form. The composition can also include compounds to improve the textural properties of the product. Other ingredients can include binders which contribute to the ease of formation and general quality of the compositions. Non-limiting examples of binders include starches, natural gums, pregelatinized starches, gelatin, polyvinylpyrrolidone, methylcellulose, sodium carboxymethylcellulose, ethylcellulose, polyacrylamides, polyvinyloxoazolidone, or polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutically active components may fit within the hydrophobic cavity, thereby producing an inclusion complex, which is soluble in water. Accordingly, the formation of the inclusion complex permits very insoluble and/or unstable pharmaceutically active components to be dissolved in water. A particularly desirable example of such agents are cyclodextrins, which are cyclic carbohydrates derived from starch. Other similar substances, however, are considered well within the scope of the present invention.

In certain embodiments, coloring agents may be added. Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), or 2-dodecenal (citrus, mandarin), combinations thereof and the like.

The sweeteners may be chosen from the following non-limiting list: saccharides, glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the compositions. These components aid in the removal of air, such as entrapped air, from the compositions. Such entrapped air may lead to non-uniform compositions. Simethicone is one particularly useful anti-foaming and/or de-foaming agent. The present invention, however, is not so limited and other suitable anti-foam and/or de-foaming agents may be used. Simethicone and related agents may be employed for densification purposes. More specifically, such agents may facilitate the removal of voids, air, moisture, and similar undesired components, thereby providing denser and thus more uniform compositions. Agents or components which perform this function can be referred to as densification or densifying agents. As described above, entrapped air or undesired components may lead to non-uniform compositions.

Any other optional components described in commonly assigned U.S. Pat. No. 7,425,292 and U.S. Pat. No. 8,765,167, referred to above, each of which is incorporated by reference in its entirety, also may be included in the compositions described herein.

The compositions further desirably contain a buffer so as to control the pH of the composition. Any desired level of buffer may be incorporated into the composition so as to provide the desired pH level encountered as the pharmaceutically active component is released from the composition. The buffer is preferably provided in an amount sufficient to control the release from the composition and/or the absorption into the body of the pharmaceutically active component. In some embodiments, the buffer may include sodium citrate, citric acid, bitartrate salt and combinations thereof.

Epinephrine prodrugs were evaluated. In vitro inhalation models were employed. In particular, an esterase hydrolysis assay was employed using human branchoalveolar lavage (BAL) fluid to examine prodrug stability. A permeability assay using in vitro a 3D culture model (EpiAirways, MatTek) of human lung epithelial cells was also performed. The 3D culture system mimics the structure of lungs, allowing for study of the hydrolysis of prodrugs in the lungs by esterases and the systemic permeability data. The EpiAirway 3D culture includes a ciliated apical surface, mucociliary epithelium and microporous membrane.

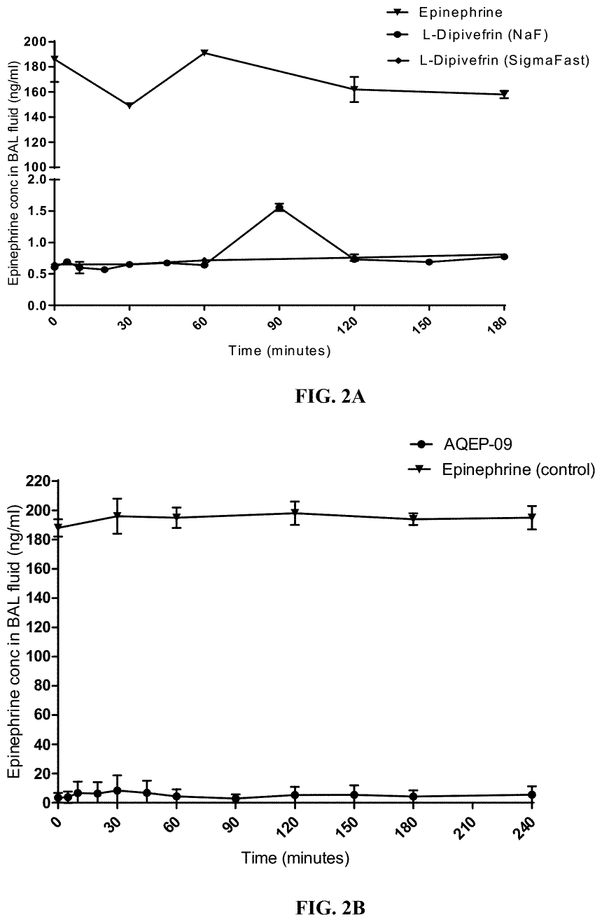

Hydrolysis of dipivefrin or AQEP-09 was studied in vitro in Human BAL fluid by measuring the epinephrine concentration. Frozen BAL fluid was used from 2 subjects. Compounds were incubated at 1 μM in the presence of a stabilizer (sodium metabisulfite, 8.9 mM). Samples were removed after stopping the enzyme reaction using NaF or SigmaFast at different timepoints. Samples were extracted and analyzed by LC-MS method. Dipivefrin and AQEP-09 showed minimal hydrolysis to epinephrine. See FIGS. 2A-2B. Less than 5% hydrolysis was observed.

In vitro permeability of the 3D culture was studied.

A Franz diffusion cell is an apparatus used for ex vivo tissue permeation assay used in the formulation development to identify the most active permeation enhancer. The Franz diffusion cell apparatus consists of two chambers separated by a membrane. The permeation studies were conducted using the 3D lung cell culture. The tissue membrane separates the donor compartment containing the prodrug and the receptor compartment containing the collection media whish was selected to provide sink conditions throughout the experiment. The permeation rate was observed over several hours by analysing drug concentration in the receptor medium.

Compounds were incubated at different concentrations (5 mM, 10 mM, and 50 mM). Cell viability was assessed by LDH Cytotoxicity Detection kit (Takara # MK401) and MTT tissue viability assay kit (MatTek, # MTT-100). Receiver media were removed at different timepoints, 0.5, 1, 2, 3, 4, 6 hrs to test permeability; Samples were extracted and analyzed by an LC-MS method.

No toxicity was seen with epinephrine, however, cell viability was reduced at all doses of Dipivefrin by the MTT assay. In the LDH assay, cell viability was reduced at all doses of Dipivefrin at 6 h, but viability was improved at 24 hr.

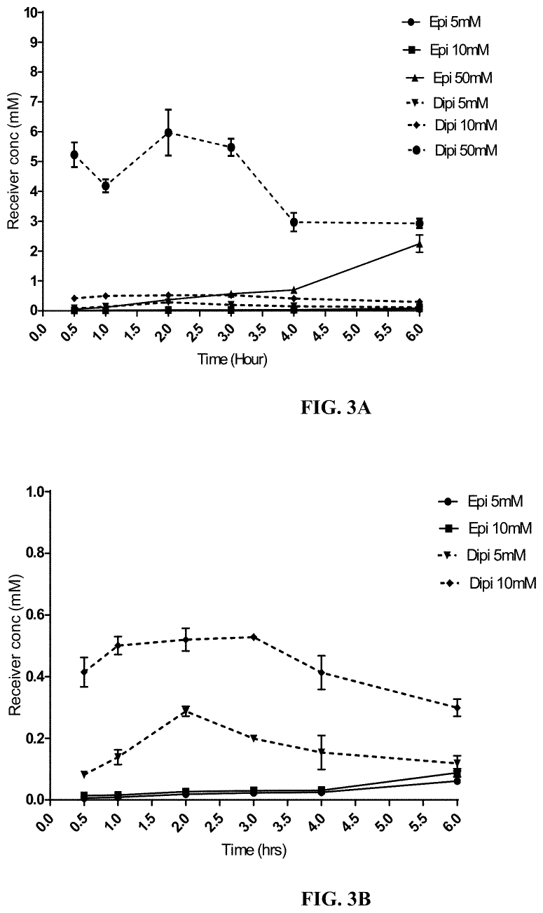

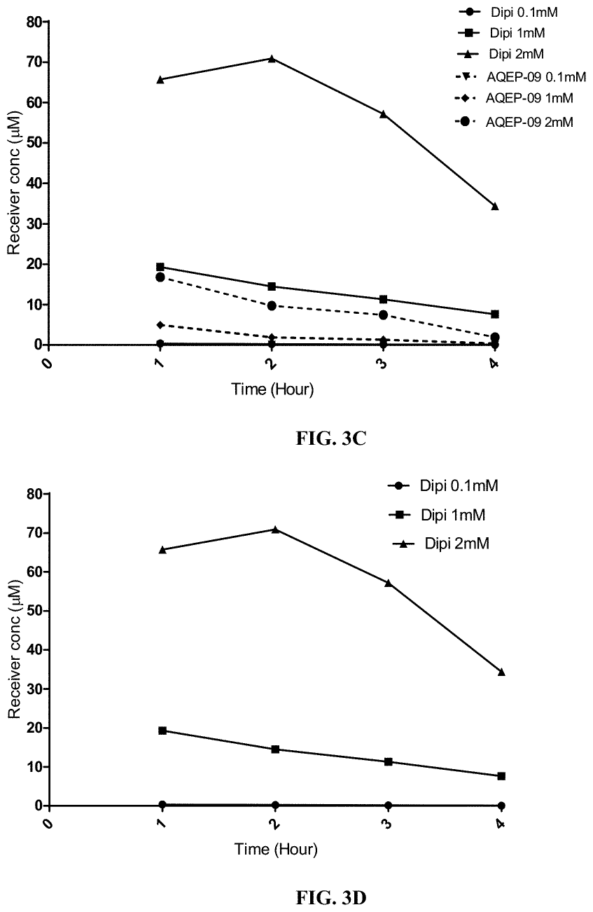

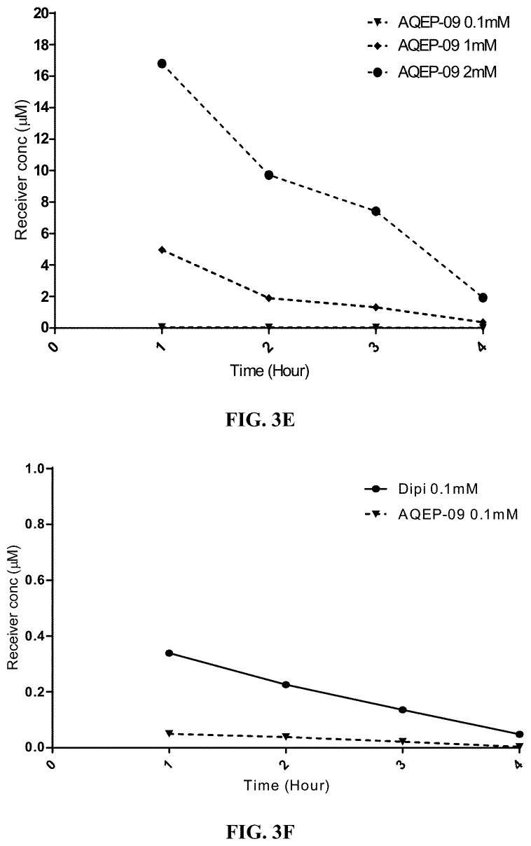

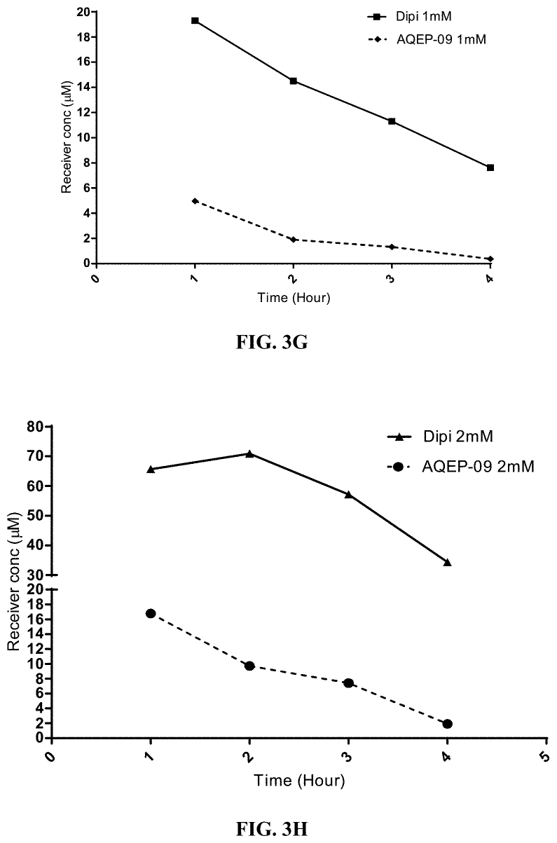

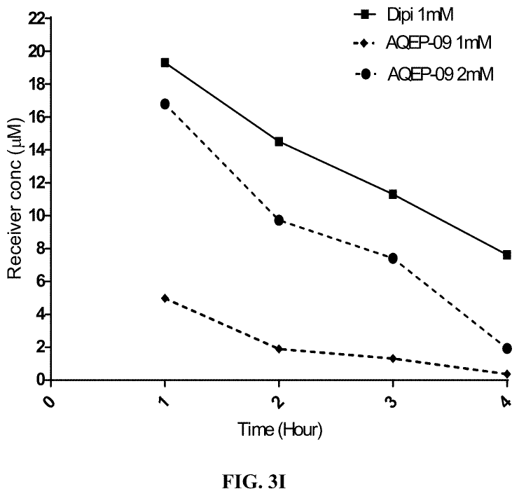

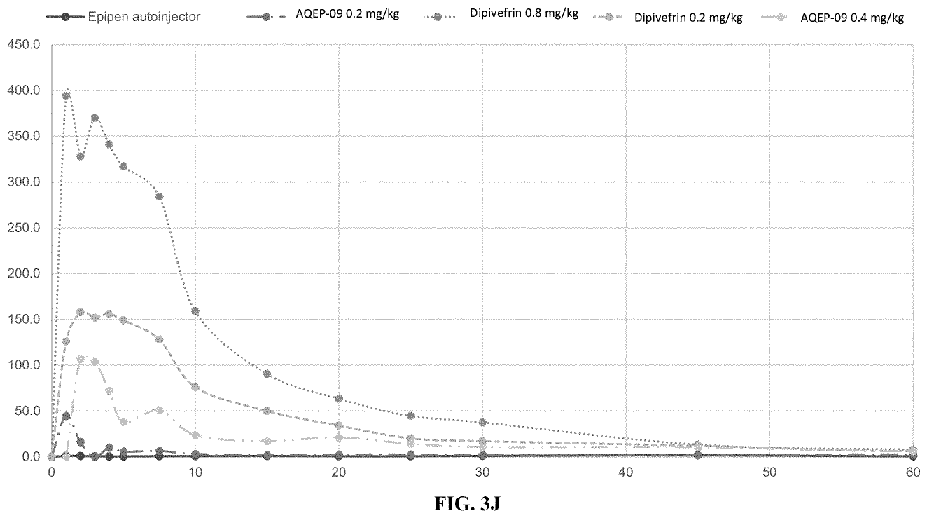

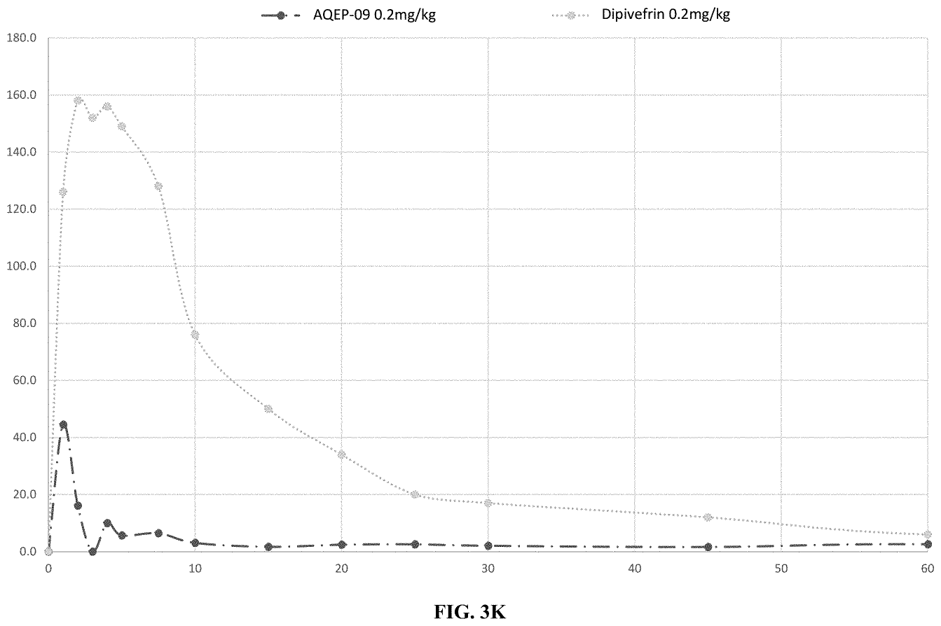

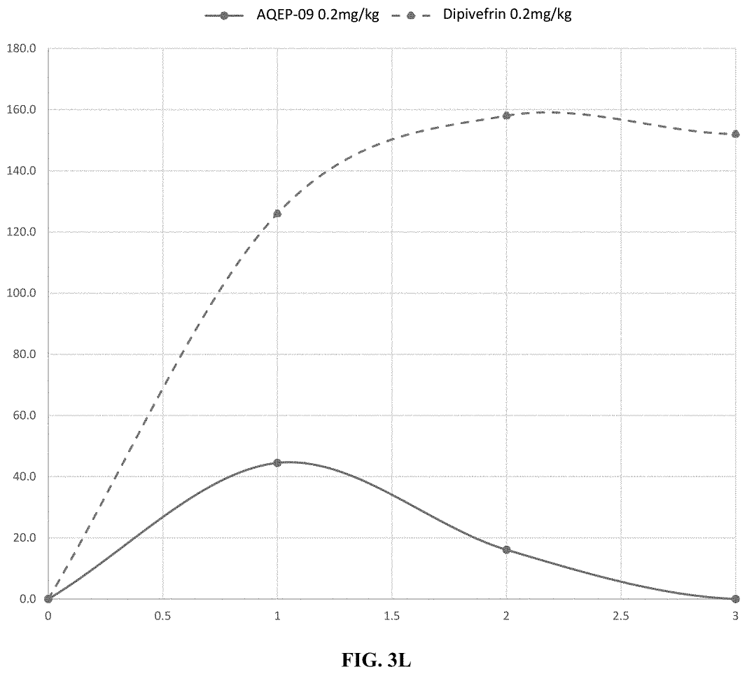

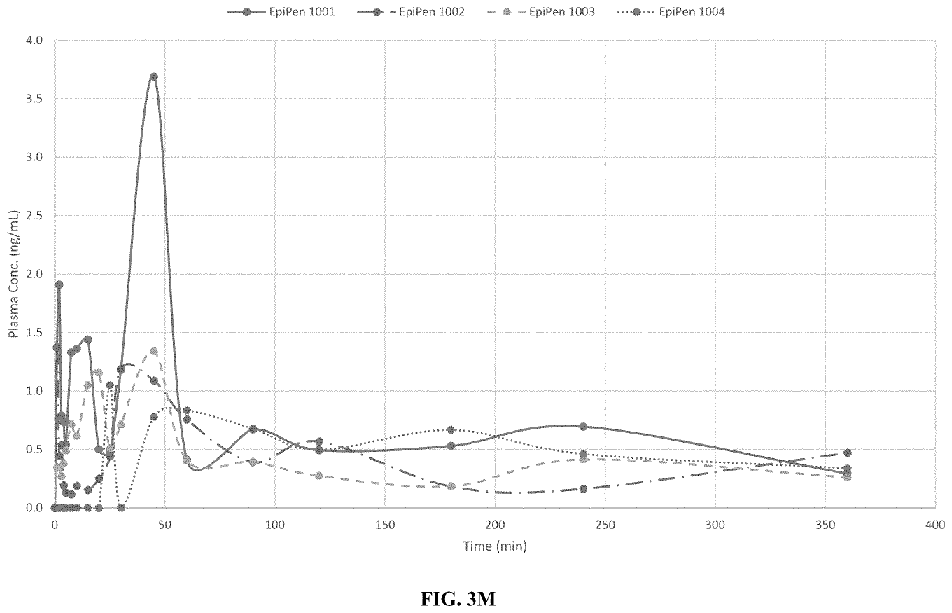

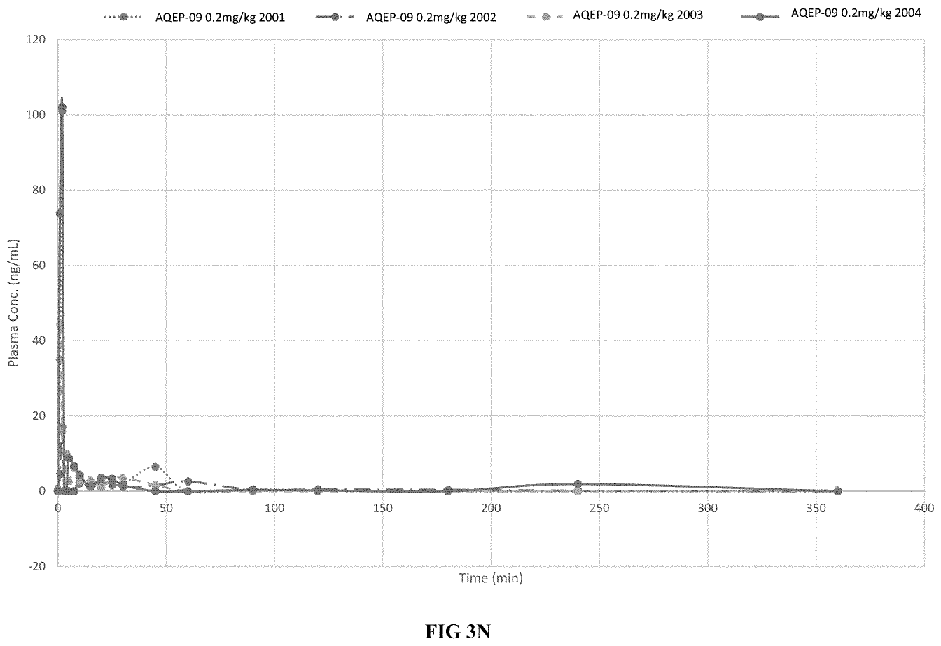

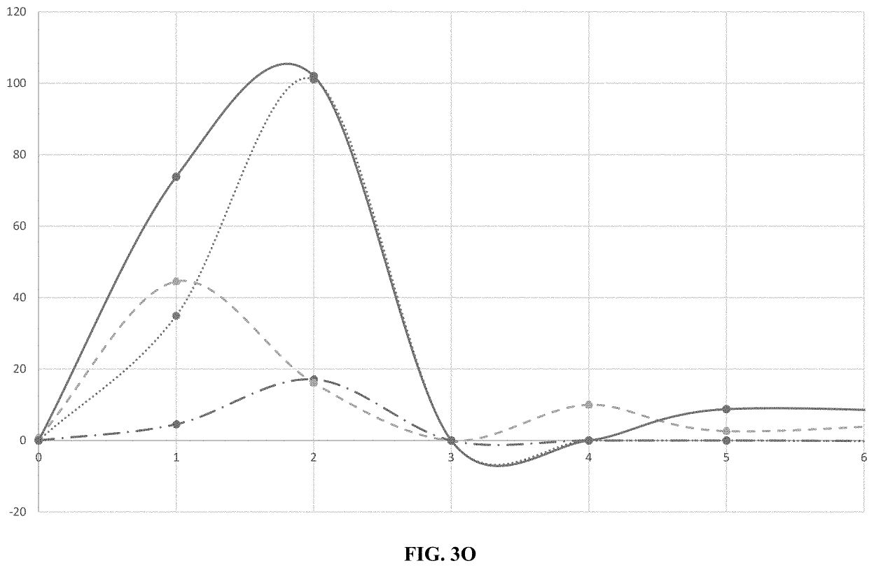

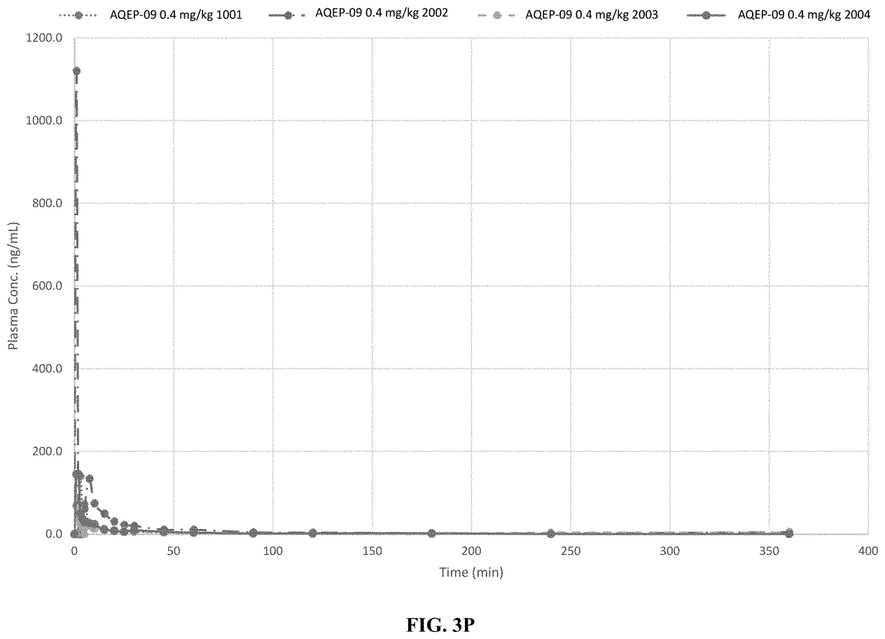

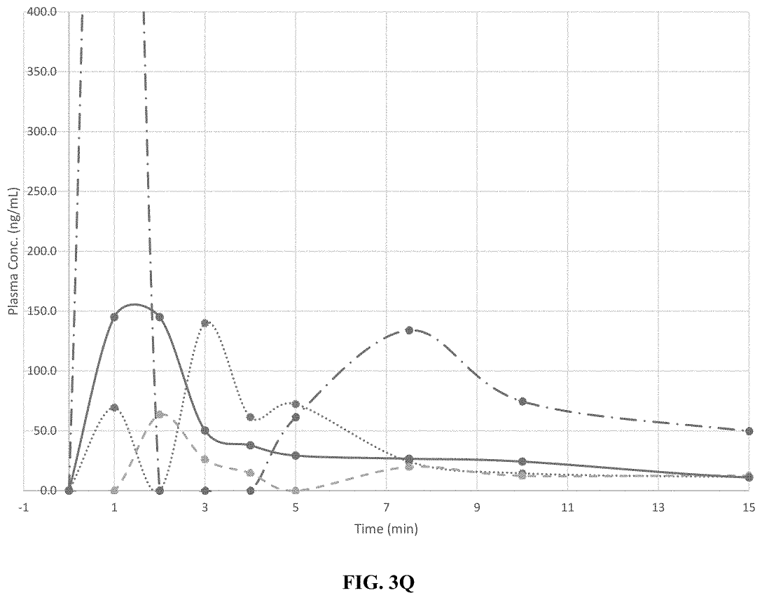

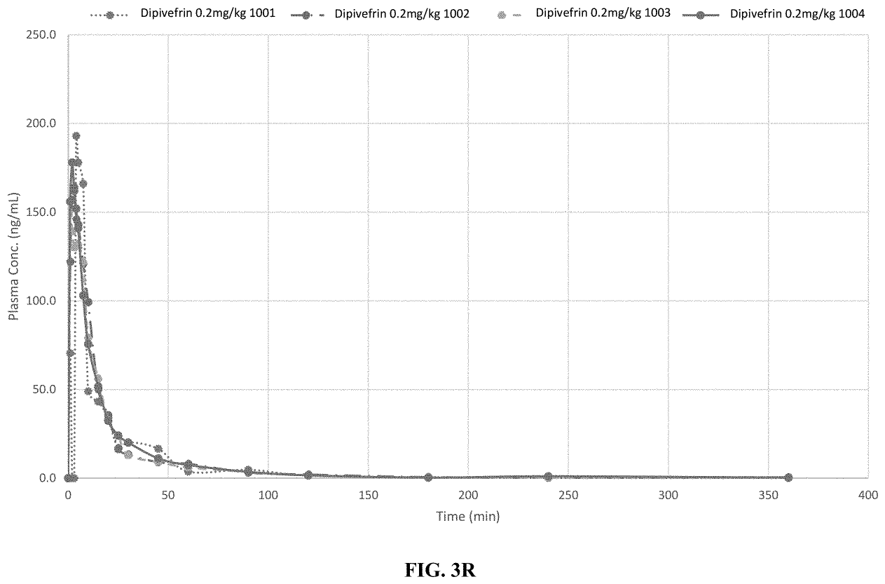

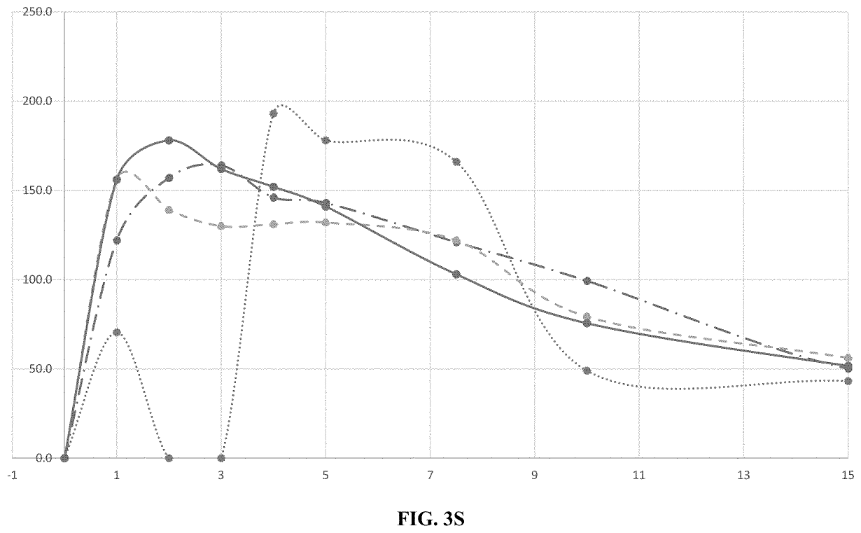

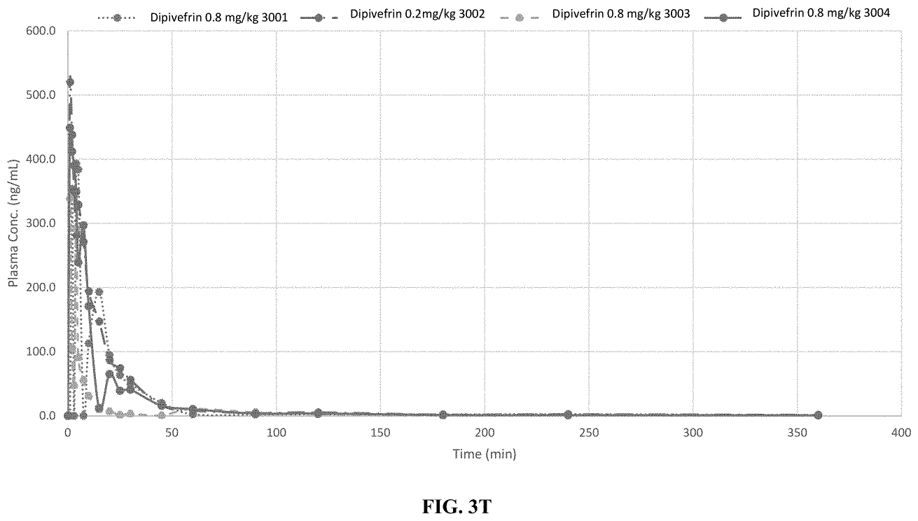

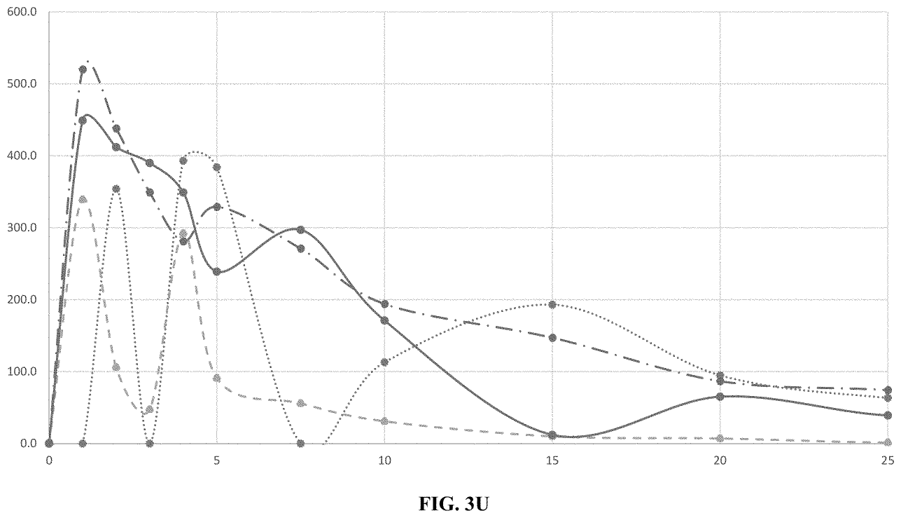

Referring to FIG. 3A and 3B, permeability studies were conducted in a human lung model. Concentration and time course was established. As indicated in the figures, epinephrine showed no significant permeability with increasing concentration, however, dose dependent permeability of Dipivefrin was observed.

Referring to FIGS. 3C-3D Dipivefrin and AQEP-09 toxicity and permeability using human lung epithelial cells was also studied in the EpiAirway model. Compounds were incubated at different concentrations (0.1 mM, 1 mM, 2 mM) and cell viability was assessed by LDH release assay and MTT assay. See, e.g., Aysun Adan, Yağmur Kiraz and Yusuf Baran, “Cell Proliferation and Cytotoxicity Assays”, Current Pharmaceutical Biotechnology (2016) 17: 1213, which is incorporated by reference in its entirety. Receiver media were removed at different timepoints, 1, 2, 3, 4 hrs to test permeability. Samples were extracted and analyzed by LC-MS method.

AQEP-09 did not show any toxicity in the MTT assay or the LDH assay, whereas lower cell viability was observed for Dipivefrin. AQEP-09 showed lower permeability at all doses tested, compared to Dipivefrin. See, FIGS. 3C-3I.

In summary, ester prodrugs of epinephrine, such as Dipivefrin and AQEP-09, showed minimal hydrolysis to epinephrine in BAL fluid. Epinephrine did not permeate even at 5-10 fold higher concentration than ester prodrugs. AQEP-09 showed lower permeability in the lung epithelial cell model, compared to Dipivefrin. Both MTT and LDH release data showed no or less toxicity by AQEP-09 at all concentrations tested compared to Dipivefrin. Since AQEP-09 is less toxic, increasing the dose may potentially help to achieve higher drug permeability (50% increase in dose shows comparable permeability to Dipivefrin).

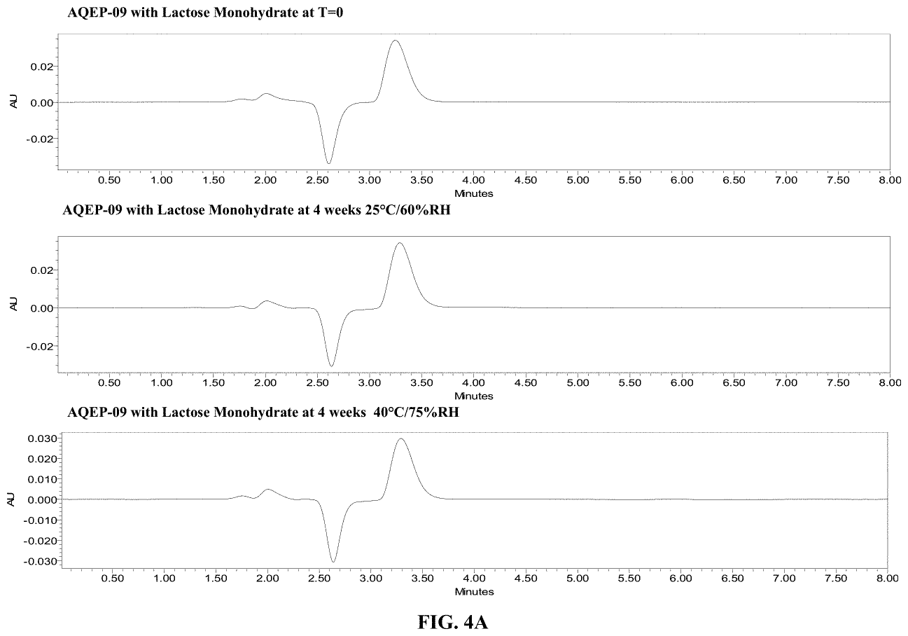

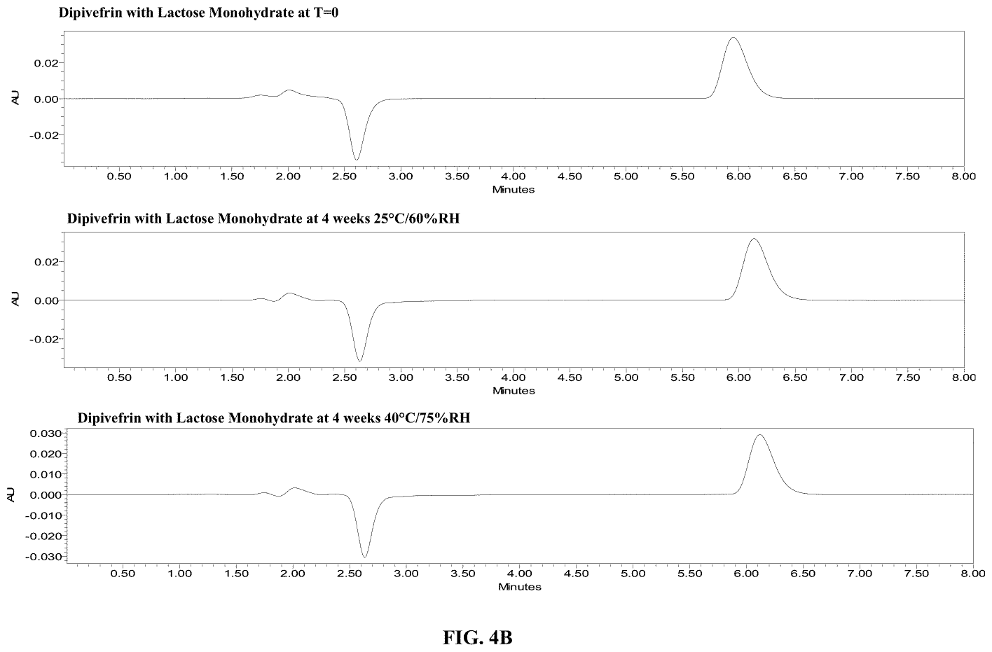

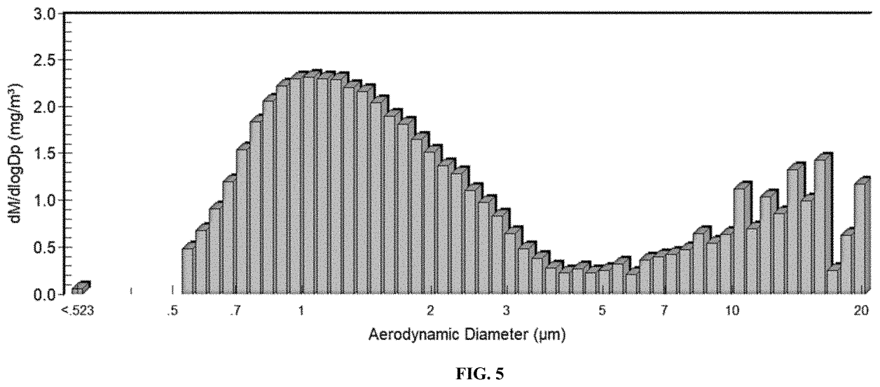

Referring to FIGS. 3J-3U, epinephrine plasma levels in beagles after deep lung delivery of epinephrine products are shown. Referring to FIG. 4A-B, the chromatograms of the prodrugs and lactose monohydrate are shown, as discussed in Examples 1 and 2 below.

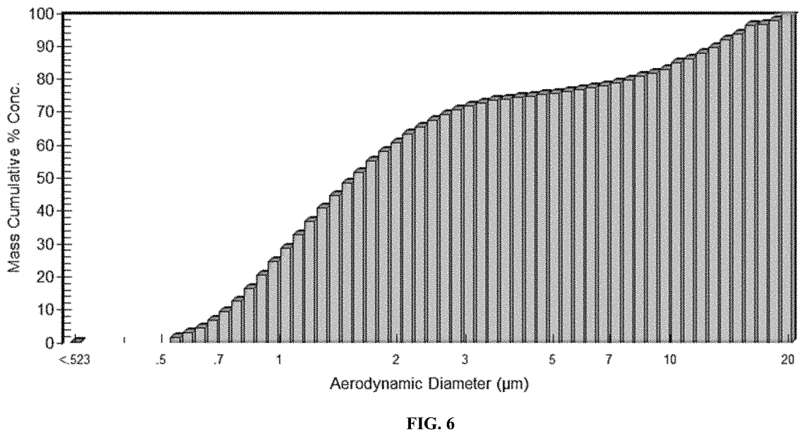

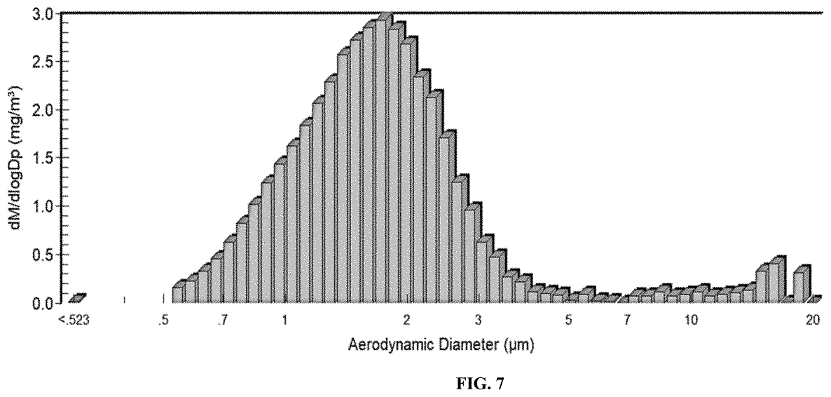

Referring to FIGS. 5 and 6, the particle size distribution for a micronized powder of dipivefrin is shown.

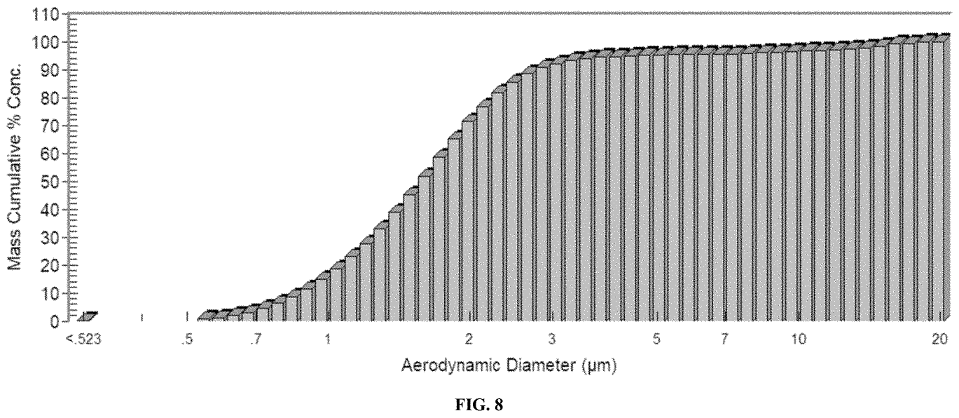

Referring to FIGS. 7 and 8, the particle size distribution for a micronized powder of AQEP-09 is shown.

### EXAMPLES

### Example 1

### Micronisation of Epinephrine Prodrugs

Both dipivefrin and Diisobutyryl L-epinephrine were micronized using the jet milling process using nitrogen. The effect of pressure and feed rates were determined. The particle sizes of the micronized material are shown in the Table 1 and micronized/milled material suitable for drug powder inhalation (2-5 μm) were manufactured.

TABLE 1.1
Particle size distribution of micronized epinephrine
prodrugs for inhalation

| | Nitrogen milling | | | |
|---|---|---|---|---|
| API | pressure | Feed rate | Particle size | |
| Dipivefrin | 1.5 bar | 840 g/hr | d10-0.91 | μm |
| | | | d50-6.24 | μm |
| | | | d90-13.18 | μm |
| Dipivefrin | 3.5 bar | 840 g/hr | d10-0.53 | μm |
| | | | d50-1.27 | μm |
| | | | d90-2.92 | μm |
| AQEP-09 | 1.5 bar | 840 g/hr | d10-10.30 | μm |
| | | | d50-55.70 | μm |
| | | | d90-132.75 | μm |
| AQEP-09 | 3.5 bar | 840 g/hr | d10-0.46 | μm |
| | | | d50-1.12 | μm |
| | | | d90-2.37 | μm |

### Example 2

### Particle Size Distribution

The MMAD and GSD for the exposure aerosol as measured by Aerodynamic Particle Sizer is presented in Table . The aerodynamic particle size distribution for the Dipivefrin and AQEP-09 are shown in FIGS. 5-8. Mass median aerodynamic diameter (MMAD) of AQEP-09 was 1.57 μm (Geometric Standard Deviation (GSD) 1.74) and MMAD for Dipivefrin was 1.47 μm (GSD 2.63). As shown in FIG. 7, about 50] percent of the particles were less than 1.47 μm (GSD 2.63), about 62 percent were less than 2 μm, and about 74 percent were less than 3 μm.

TABLE 2.1
Summary of Particle Size Distribution

| Exposure Group | MMAD (μm) | GSD |
|---|---|---|
| Dipivefrin | 1.47 | 2.63 |
| AQEP-09 | 1.57 | 1.74 |

### Example 3

### Excipient Compatibility Study

Compatibility of epinephrine prodrugs Disobutyryl L-epinephrine (AQEP-09) and Dipivefrin was studied with common carriers used for dry powder inhalation applications. Lactose monohydrate, Lactose anhydrous and Mannitol were used for the stability evaluation. Mixtures of the micronized API and the carriers were tested under stability conditions, 25° C./60%RH and 40° C./75%RH. The data is presented in the Table 3.1 and Table 3.2. Preliminary evaluation suggest that they are stable at room temperature (shelf-life conditions) for at least 4 weeks. The chromatograms shown in FIG. 4A demonstrate the chromatographic profile (single active peak) of AQEP-09 in the presence of lactose monohydrate. The chromatograms shown in FIG. 4B demonstrate the chromatographic profile (single active peak) of Dipivefrin in the presence of lactose monohydrate.

TABLE 3.1
Compatibility of Diisobutyryl L-Epinephrine with
dry powder inhalation carriers

| | | | |
|---|---|---|---|
| Time (Weeks) | Lactose Monohydrate | Lactose Anhydrous | Mannitol |

Assay (Percent Label Claim)-25° C./60% RH

| | | | |
|---|---|---|---|
| 0 | 100% | 100% | 100% |
| 2 | 100% | 103% | 96% |
| 4 | 97% | 103% | 96% |

Assay (Percent Label Claim)-40° C./75% RH

| | | | |
|---|---|---|---|
| 0 | 100% | 100% | 100% |
| 2 | 94% | 97% | 95% |
| 4 | 83% | 87% | 84% |

TABLE 3.2
Compatibility of Dipivefrin with dry powder inhalation carriers

| | | | |
|---|---|---|---|
| Time (Weeks) | Lactose Monohydrate | Lactose Anhydrous | Mannitol |

Assay (Percent Label Claim)-25° C./60% RH

| | | | |
|---|---|---|---|
| 0 | 100% | 100% | 100% |
| 2 | 101% | 100% | 101% |
| 4 | 96% | 96% | 98% |

Assay (Percent Label Claim)-40° C./75% RH

| | | | |
|---|---|---|---|
| 0 | 100% | 100% | 100% |
| 2 | 96% | 99% | 101% |
| 4 | 81% | 95% | 95% |

### Example 4

### Beagle Study

A study was completed to evaluate the pharmacokinetics (PK) of epinephrine after bolus inhalation of AQEP-09 and Dipivefrin in Beagle Dogs. Bolus inhalation was compared to intramuscular (IM) delivery of epinephrine. Prior to this study, there was insufficient dog toxicology data to support the clinical use of the proposed prodrugs via inhalation. Bolus inhalation in dogs covered the route(s) of administration of these TAs in the proposed clinical trials.

The experimental design for this study is shown below. Twelve (12) beagle dogs were randomized among 3 groups. Animals in Group 1 received a single intramuscular (IM) dose of epinephrine (EpiPen® autoinjector, 0.3 mg). Animals in Group 2 and Group 3 received a single dose of AQEP-09 or Dipivefrin prodrugs, respectively via bolus inhalation. Blood for PK analysis was collected post dosing. Bronchoalveolar lavage was collected once post exposure.

Animals in Groups 1 and 2 were subjected to a washout period of at least 7 days. In the second arm of the study, animals in Group 1 were dosed with Dipivefrin and animals in Group 2 were dosed with AQEP-09 then have blood and Bronchoalveolar lavage (BAL) sampling as performed in the first arm of the study. The test article (TA) was delivered in anesthetized dogs via endotracheal tube as a dry powder utilizing the Lovelace Bolus Delivery system. Apnea was induced in the anesthetized animal, the insufflator attached to the expansion chamber and endotracheal tube and the aerosol “puffed” into the lungs with positive pressure using an Ambubag and a syringe. Once the aerosol was delivered, the animal was recovered from anesthesia.

| | | | | | | Blood | BAL |
|---|---|---|---|---|---|---|---|
| Arm | Group | Exposure | Route | Target Dose | Animals | Collection | Collection |
| Arm 1 | 1 | Epipen ® | IM | 0.3 mg | 2 Male, | 0 (prior to | na ^{D} |
| | | autoinjector^{A} | | | 2 Female | dose), 1, 2, | Left and |
| | 2 | AQEP-09 | Bolus | 2 insufflators, | 4 Female | 3, 4, 5, 7.5, | Right Caudal |
| | | | inhalation | 0.2 mg/kg ^{B} | | 10, 15, 20, | lobes at |
| | 3 | Dipivefrin | Bolus | 8 insufflators, | 4 Female | 25, 30, 45, | 360 min post |
| | | | inhalation | 0.8 mg/ kg ^{B} | | 60, 90, 120, | dose |
| Arm 2 | 1 | Dipivefrin ^{C} | Bolus | 2 insufflators, | 2 Male, | 180, 240, | Left and |
| | | | inhalation | 0.2 mg/kg | 2 Female | 360 | Right Caudal |
| | 2 | AQEP-09 ^{C} | Bolus | 4 insufflators, | 4 Female | minutes | lobes at |
| | | | inhalation | 0.4 mg/kg | | post | 360 min |
| | | | | | | dosing | post dose |

Dose was delivered using EpiPen ®, 0.3 mg (Mylan, Canonsburg, PA).
^{B} In terms of presented doses (mg of prodrug freebase); assumed a 10 kg dog. In Arm 1, animals in Group 2 and Group 3 received a single dose of AQEP-09 or Dipivefrin, respectively via bolus inhalation
^{C} Animals in Groups 1 and 2 underwent a washout period of at least 7 days Animals in Group 1 were dosed with dipivefrin in the second arm of the study. In Arm 2 of the study, animals in Group 2 were dosed with AQEP-09.
^{D} For each Arm, BAL was collected from animals in AQEP-09 and Dipivefrin groups only.

The insufflator devices were weighed prior to and after delivery to determine the net weights to provide the amount of delivered material. Testing and exposures were conducted with nominal insufflator load weight of 10±1.5 mg of each respective test article loaded into the reservoir of the insufflator. The number of insufflators used were varied to achieve target doses. Two insufflators were used per animal for low dose exposures. The high dose dipivefrin exposure used 8 insufflators and was reduced to 4 insufflators. Aerosol data for Low and High Dose AQEP-09 exposures is summarized in Table 4.1 and Table 4.2, respectively; and data for Low and High Dose Dipivefrin exposures is summarized in Table 4.3 and Table 4.4, respectively. The average amount of material ejected during Low and High Dose AQEP-09 exposures was 12.92 and 23.32 mg, respectively. The average amount of material ejected during Low and High Dose Dipivefrin exposures was 16.56 and 54.44 mg, respectively.

TABLE 4.1
Low Dose AQEP-09 Exposure Summary (Arm 1, Group 2)

| | | Total | Total | Total | AQEP- | AQEP- | AQEP-09 |
|---|---|---|---|---|---|---|---|
| | Body | Amount | Amount | Amount | 09 | 09 | Deposited |
| Animal | Weight | Ejected | Presented | Deposited | Presented | Deposited | Dose |
| ID | (kg) | (mg) | (mg) | (mg) | (mg) | (mg) | (mg/kg) |
| 2001 | 8.7 | 13.58 | 5.50 | 1.65 | 4.35 | 1.30 | 0.15 |
| 2002 | 9.4 | 12.68 | 5.13 | 1.54 | 3.16 | 0.95 | 0.13 |
| 2003 | 7.8 | 12.55 | 6.30 | 1.89 | 4.98 | 1.49 | 0.19 |
| 2004 | 7.2 | 9.88 | 4.00 | 1.20 | 3.16 | 0.95 | 0.13 |

| | | | | | | |
|---|---|---|---|---|---|---|
| Average | 12.92 | 5.23 | 1.57 | 3.91 | 1.17 | 0.15 |
| SD | 2.36 | 0.96 | 0.29 | 0.90 | 0.27 | 0.03 |

TABLE 4.2
High Dose AQEP-09 Exposure Summary (Arm 2, Group 2)

| | | | | | | | AQEP- |
|---|---|---|---|---|---|---|---|
| | | Total | Total | Total | AQEP- | AQEP- | 09 |
| | Body | Amount | Amount | Amount | 09 | 09 | Deposited |
| Animal | Weight | Ejected | Presented | Deposited | Presented | Deposited | Dose |
| ID | (kg) | (mg) | (mg) | (mg) | (mg) | (mg) | (mg/kg) |
| 2001 | 9.1 | 24.83 | 10.18 | 3.05 | 8.04 | 2.41 | 0.27 |
| 2002 | 9.3 | 22.14 | 9.08 | 2.72 | 7.17 | 2.15 | 0.23 |
| 2003 | 7.8 | 25.32 | 10.38 | 3.11 | 8.20 | 2.46 | 0.32 |
| 2004 | 7.4 | 20.99 | 8.60 | 2.58 | 6.80 | 2.04 | 0.28 |

| | | | | | | |
|---|---|---|---|---|---|---|
| Average | 23.32 | 9.56 | 2.87 | 7.55 | 2.27 | 0.27 |
| SD | 2.09 | 0.86 | 0.26 | 0.68 | 0.20 | 0.03 |

TABLE 4.3
Low Dose Dipivefrin Exposure Summary (Arm 2, Group 1)

| | | Total | Total | Total | | | Dipivefrin |
|---|---|---|---|---|---|---|---|
| | Body | Amount | Amount | Amount | Dipivefrin | Dipivefrin | Deposited |
| Animal | Weight | Ejected | Presented | Deposited | Presented | Deposited | Dose |
| ID | (kg) | (mg) | (mg) | (mg) | (mg) | (mg) | (mg/kg) |
| 1001 | 9.1 | 14.17 | 2.63 | 0.79 | 2.29 | 0.69 | 0.08 |
| 1002 | 7.9 | 16.45 | 3.05 | 0.92 | 2.66 | 0.80 | 0.10 |
| 1003 | 7.6 | 17.64 | 3.27 | 0.98 | 2.85 | 0.85 | 0.11 |
| 1004 | 7.5 | 17.99 | 3.34 | 1.00 | 2.90 | 0.87 | 0.12 |

| | | | | | | |
|---|---|---|---|---|---|---|
| Average | 16.56 | 3.07 | 0.92 | 2.67 | 0.80 | 0.10 |
| SD | 1.73 | 0.32 | 0.010 | 0.28 | 0.08 | 0.02 |

TABLE 4.4
High Dose Dipivefrin Exposure Summary (Arm 1, Group 3)

| | | Total | Total | Total | | | Dipivefrin |
|---|---|---|---|---|---|---|---|
| | Body | Amount | Amount | Amount | Dipivefrin | Dipivefrin | Deposited |
| Animal | Weight | Ejected | Presented | Deposited | Presented | Deposited | Dose |
| ID | (kg) | (mg) | (mg) | (mg) | (mg) | (mg) | (mg/kg) |
| 3001 | 8.7 | 49.22 | 9.13 | 2.74 | 7.95 | 2.38 | 0.27 |
| 3002 | 7.5 | 62.34 | 11.57 | 3.47 | 10.06 | 3.02 | 0.40 |
| 3003 | 8.1 | 53.65 | 9.96 | 2.99 | 8.66 | 2.60 | 0.32 |
| 3004 | 8.8 | 52.56 | 9.75 | 2.93 | 8.49 | 2.55 | 0.29 |

| | | | | | | |
|---|---|---|---|---|---|---|
| Average | 54.44 | 10.10 | 3.03 | 8.79 | 2.64 | 0.32 |
| SD | 5.59 | 1.04 | 0.31 | 0.90 | 0.27 | 0.06 |

For AQEP-09 and Dipivefrin, doses were calculated as presented and deposited dose by sex and sexes combined. The total presented and pulmonary deposited dose on a mg/kg basis are included in the tables above. For AQEP-09 average presented doses were 3.91 and 7.55 mg/kg and average delivered doses were 1.17 and 2.27 mg/kg or the Low and High Dose groups, respectively. For Dipivefrin average presented doses were 2.67 and 8.79 mg/kg and average delivered doses were 0.80 and 2.64 mg/kg or the Low and High Dose groups, respectively.

For the Epinephrine group, dose was determined by dividing the amount (mg) delivered via IM EpiPen® dosing by the animals bodyweight in kilograms (Table 4.5). The average epinephrine dose was 0.037 mg//kg.

TABLE 4.5
Epinephrine (EpiPen ®) Dosing Summary (Arm
1, Group 1)

| | Body | | |
|---|---|---|---|
| Animal | Weight | Total Amount | Dose |
| ID | (kg) | Delivered(mg) | (mg/kg) |
| 1001 | 9.6 | 0.3 | 0.031 |
| 1002 | 8.4 | 0.3 | 0.036 |
| 1003 | 7.9 | 0.3 | 0.038 |
| 1004 | 7.4 | 0.3 | 0.041 |

| | |
|---|---|
| Average | 0.037 |

Presented and deposited doses were calculated from the amount of material ejected from insufflators, delivery efficiency, and drug composition percentage of the TA. The overall efficiencies for each TA were determined prior to exposures and were 0.41 for AQEP-09, and 0.19 for dipivefrin.

The average calculated presented doses for AQEQ-09 were 0.50 mg/kg (standard deviation 0.09) for the low dose, and 0.91 mg/kg (standard deviation 0.11) for the high dose. The average calculated presented doses for dipivefrin were 0.34 mg/kg (standard deviation 0.06) for the low dose and 1.07 mg/kg (standard deviation 0.19) for the high dose. Presented and deposited doses are summarized below. The amount presented is the amount ejected from the syringe. The amount deposited is the amount that reaches the endotracheal tube.

| | | Presented | Deposited |
|---|---|---|---|
| Group | Exposure | Dose (mg/kg) | Dose (mg/kg) |
| 1 | Low Dose Dipivefrin | 0.34 ± 0.06 | 0.10 ± 0.02 |
| 2 | Low Dose AQEP-09 | 0.50 ± 0.09 | 0.15 ± 0.03 |
| 2 | High Dose AQEP-09 | 0.91 ± 0.11 | 0.27 ± 0.03 |
| 3 | High Dose Dipivefrin | 1.07 ± 0.19 | 0.32 ± 0.06 |

± values are standard deviation

Referring to FIG. 3J, the results of an inhalation study are presented reflecting the epinephrine plasma levels in beagles after deep lung delivery of the indicated prodrugs. The dotted and dashed lines reflect prodrug data as compared to Epipen® autoinjector data

Referring to FIG. 3K, the results of prodrugs are magnified. Referring to FIG. 3K and 3I, data reflecting the consistency of results between in-vitro and Beagle model are shown. There is about a four-fold difference in permeation.

Referring to FIG. 3M, the Epipen® profile in dogs is provided for reference. Plasma concentrations of greater than 1 and less than 4.0 ng/ml are shown and reflected in the chart below. For example, greater than 1.2, greater than 1.3, greater than 1.5, and greater than 1.8. EpiPen® was dosed at 0.3 mg/animal resulting in 0.03-0.05 mg/kg.

EpiPen ®

| 0.3 mg | 1001 | 1002 | 1003 | 1004 | Mean |
|---|---|---|---|---|---|
| AUC | 257 | 144.4 | 144.2 | 187.4 | 183.25 |
| Cmax | 3.69 | 1.38 | 1.34 | 1.05 | 1.865 |
| 9 ng/ml) | | | | | |
| Tmax (min) | 45 | 30 | 45 | 25 | 37.5 |

Referring to FIG. 3N to 3U, the Epinephrine profiles via inhalation in beagles are shown and reflected in the chart below.

FIG. 3N shows the epinephrine profiles from AQEP-09 (0.2mg/kg) via inhalation. FIG. 3O is an enlarged inset graph of FIG. 3N. FIG. 3P shows epinephrine profiles from AQEP-09 (0.4mg/kg) via inhalation. FIG. 3Q is an enlarged inset graph of FIG. 3P. As noted below, the Cmax is greater than 100 mg/kg, greater than 120 mg/kg, greater than 130 mg/kg, and greater than 140 mg/kg, greater than 200 mg.kg, greater than 300 mg/kg, greater than 500 mg/kg, greater than 1000 mg/kg, and less than 1500 mg/kg.

FIG. 3R shows epinephrine profiles from Dipivefrin (0.2mg/kg) via inhalation. FIG. 3S is an enlarged inset graph of FIG. 3R.

FIG. 3T shows Epinephrine profiles from Dipivefrin (0.8mg/kg) via inhalation. FIG. 3U is an enlarged inset graph of FIG. 3T. As noted below, the Cmax is greater than 200 mg/kg, greater than 300 mg/kg, greater than 400 mg/kg and less than 1000 mg/kg.

The following pK data was obtained for prodrug AQEP-09. Dog 2002 had a significantly different dose response than the other 3 dogs. Overall dose response slightly less than dose proportional (target 2×).

AQEP-09

| 0.4 mg/kg | 2001 | 2002 | 2003 | 2004 | Mean |
|---|---|---|---|---|---|
| AUC | 1188 | 5583 | 1210 | 1300 | 2320.25 |
| Cmax (ng/ml) | 140 | 1120 | 63.5 | 145 | 367 |
| Tmax (min) | 3 | 1 | 2 | 1 | 1.5 |
| 0.2 mg/kg | 2001 | 2002 | 2003 | 2004 | Mean |
| AUC | 757.4 | 271.9 | 506.4 | 537.3 | 518.25 |
| Cmax (ng/ml) | 101 | 17.1 | 44.5 | 102 | 66.15 |
| Tmax (min) | 2 | 2 | 1 | 2 | 2 |
| AUC | 1.57 | 20.53 | 2.39 | 2.42 | 4.48 |
| Cmax (ng/ml) | 1.39 | 65.50 | 1.43 | 1.42 | 5.55 |

The following pK data was obtained for prodrug Dipivefrin. As shown below, 4× dose increase produced ˜2× dose response.

Dipivefrin

| 0.8 mg/kg | 3001 | 3002 | 3003 | 3004 | Mean |
|---|---|---|---|---|---|
| AUC | 5874 | 6874 | 1836 | 5125 | 4927.25 |
| Cmax (ng/ml) | 393 | 520 | 339 | 449 | 425.25 |
| Tmax (min) | 4 | 1 | 1 | 1 | 1 |
| 0.2 mg/kg | 1001 | 1002 | 1003 | 1004 | Mean |
| AUC | 2679 | 2708 | 2514 | 2755 | 2664 |
| Cmax (ng/ml) | 193 | 164 | 156 | 178 | 172.75 |
| Tmax (min) | 4 | 3 | 1 | 2 | 2.5 |

Dipivefrin Dose response

| | | | | | |
|---|---|---|---|---|---|
| AUC | 2.19 | 2.54 | 0.73 | 1.86 | 1.85 |
| Cmax (ng/ml) | 2.04 | 3.17 | 2.17 | 2.52 | 2.46 |

In certain embodiments, the composition including a prodrug is administered in a dose of greater than 0.05 mg and less than 5 mg.

In certain embodiments, the composition has a Cmax of greater than 5 and less than 300 mg/kg.

In certain embodiments, the effective plasma concentration of a pharmaceutically active form of the prodrug has a Tmax of greater than 0.5 seconds and less than 40 seconds.

Other embodiments are within the scope of the following claims.

What is claimed is:

1. A method of treating a medical condition in a human subject comprising:
administering by inhalation a composition including a prodrug, the prodrug passing through lung tissue to achieve an effective plasma concentration of a pharmaceutically active form of the prodrug in the human subject.

2. The method of claim 1, further comprising providing the composition in an inhaler.

3. The method of claim 2, wherein the composition is a powder having a mean median aerodynamic diameter of less than 5 microns.

4. The method of claim 2, wherein the composition is a liquid, gel or a suspension.

5. The method of claim 2, wherein the inhaler includes a propellant.

6. The method of claim 1, further comprising providing the composition in a nebulizer.

7. The method of claim 1, further comprising providing the composition in an aerosol.

8. The method of claim 7, wherein providing the composition in an aerosol includes rapidly heating the composition to vaporize or sublimate the composition.

9. The method of claim 1, wherein the composition consists essentially of the prodrug.

10. The method of claim 1, wherein the composition consists essentially of the prodrug and epinephrine.

11. The method of claim 1, wherein the composition consists essentially of the prodrug and a second prodrug.

12. The method of claim 1, wherein the prodrug includes an alkyl ester of a pharmaceutically active form of the prodrug.

13. The method of claim 1, wherein the prodrug includes a butyl ester of a pharmaceutically active form of the prodrug.

14. The method of claim 1, wherein the prodrug includes an isopropyl ester of a pharmaceutically active form of the prodrug.

15. The method of claim 1, wherein the prodrug includes an ethyl ester of a pharmaceutically active form of the prodrug.

16. The method of claim 1, wherein the prodrug includes an ester of epinephrine.

17. The method of claim 1, wherein the medical condition is in a spectrum of anaphylaxis.

18. The method of claim 1, wherein the medical condition is an allergic reaction.

19. The method of claim 1, wherein the medical condition is a cardiac abnormality.

20. The method of claim 1, wherein the medical condition is a pulmonary abnormality.

21. The method of claim 1, wherein the composition including a prodrug includes more than one prodrug with each prodrug being a derivative of a pharmaceutically active ingredient.

22. The method of claim 21, wherein the first prodrug is a first ester of epinephrine and the second prodrug is a second ester of epinephrine, the first ester of epinephrine and the second ester of epinephrine being different.

23. The method of claim 1, wherein the prodrug is a compound of formula (I), wherein 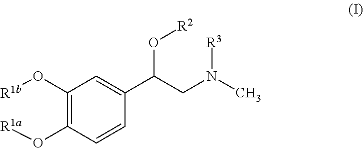
each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R¹, R² and R³ is not H, or a pharmaceutically acceptable salt thereof.

24. The method of claim 23, wherein R² and R³ are H and each R^{1a} and R^{1b}, independently, is ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl.

25. An inhalation device comprising:
a housing and a composition within the housing, the composition including a prodrug.

26. The device of claim 25, wherein the composition consists essentially of the prodrug.

27. The device of claim 25, wherein the prodrug includes an alkyl ester of a pharmaceutically active compound.

28. The device of claim 25, wherein the prodrug includes a butyl ester of a pharmaceutically active compound.

29. The device of claim 23, wherein the prodrug includes an isopropyl ester of a pharmaceutically active compound.

30. The device of claim 25, wherein the prodrug includes an ethyl ester of a pharmaceutically active compound.

31. The device of claim 25, wherein the prodrug includes an ester of epinephrine.

32. The device of claim 25, wherein the composition including a prodrug includes more than one prodrug with each prodrug being a derivative of a pharmaceutically active ingredient.

33. The device of claim 25, wherein a first prodrug is a first ester of epinephrine and a second prodrug is a second ester of epinephrine, the first ester of epinephrine and the second ester of epinephrine being different.

34. The device of claim 25, wherein the prodrug is a compound of formula (I), wherein 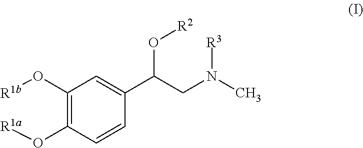
each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R¹, R² and R³ is not H, or a pharmaceutically acceptable salt thereof.

35. The device of claim 25, wherein R² and R³ are H and each R^{1a} and R^{1b}, independently, is ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl.

36. The inhalation device of claim 25, wherein the housing is a blister-based housing and the composition is a preloaded dose of a powder.

37. The inhalation device of claim 25, wherein the housing includes a capsule comprising a unit dose of a powder of the composition.

38. The composition of claim 25, wherein the composition comprises the prodrug and epinephrine.

39. The composition of claim 25, wherein the composition comprises the prodrug and a second prodrug.

40. A method of treating a medical condition in a mammal comprising administering a therapeutically effective amount of a composition comprising a prodrug and epinephrine, and delivering the composition both locally and systemically.

41. A method of treating a medical condition in a mammal comprising administering a therapeutically effective amount of a composition comprising a first prodrug and a second prodrug and delivering the composition both locally and systemically.

42. The method of claim 41, wherein the therapeutically effective amount is a ratio of a presented dose to a deposited dose.

43. The method of claim 42, wherein the ratio is greater than 0.28.

44. The method of claim 42, wherein the ratio is about 0.3.

45. The method of claim 41, wherein the amount of the first prodrug deposited is greater than 0.09 mg/kg.

46. The method of claim 41 wherein the second prodrug is deposited is greater than 0.13 mg/kg.

47. The method of claim 41, wherein the method of claim 41, wherein the amount of the first prodrug deposited is greater than 0.3 mg/kg.

48. The method of claim 41 wherein the second prodrug is deposited is greater than 0.25 mg/kg.

49. The method of claim 12 wherein the pharmaceutically active form is epinephrine.

50. The method of claim 13 wherein the pharmaceutically active form is epinephrine.

51. The method of claim 14 wherein the pharmaceutically active form is epinephrine.

52. The method of claim 15 wherein the pharmaceutically active form is epinephrine.

53. The method of claim 1, wherein the composition produces plasma levels of epinephrine greater than 0.5 mg/g and less than 450 mg/kg.

54. The method of claim 1, wherein the composition including a prodrug is administered in a dose of greater than 0.05 mg and less than 5 mg.

55. The method of claim 54 wherein the dose is about 0.5 mg.

56. The method of claim 54 wherein the dose is about 1.0 mg.

57. The method of claim 54 wherein the dose is about 1.5 mg.

58. The method of claim 54 wherein the dose is about 2.0 mg.

59. The method of claim 1, wherein the composition has a Cmax of greater than 5 and less than 300 mg/kg.

60. The method of claim 1, wherein the effective plasma concentration of a pharmaceutically active form of the prodrug has a Tmax of greater than 0.5 seconds and less than 40 seconds.
